(19) United States
(12) Patent Application Publication
(10) Pub. No.: US 2013/0178413 A1
(43) Pub. Date: Jul. 11, 2013

(54) HCV NS3 PROTEASE INHIBITORS

(71) Applicants: John A. McCauley, Maple Glen, PA (US); Nigel J. Liverton, Harleysville, PA (US); Michael T. Rudd, Collegeville, PA (US); Kevin F. Gilbert, Barto, PA (US); Marco Ferrara, Milanese (IT); Vincenzo Summa, Roma (IT); Benedetta Crescenzi, Paris (FR)

(72) Inventors: John A. McCauley, Maple Glen, PA (US); Nigel J. Liverton, Harleysville, PA (US); Michael T. Rudd, Collegeville, PA (US); Kevin F. Gilbert, Barto, PA (US); Marco Ferrara, Milanese (IT); Vincenzo Summa, Roma (IT); Benedetta Crescenzi, Paris (FR)

(21) Appl. No.: 13/825,245

(22) PCT Filed: Sep. 16, 2011
(86) PCT No.: PCT/US11/51871

§ 371 Date: Mar. 20, 2013

Related U.S. Application Data
(60) Provisional application No. 61/384,855, filed on Sep. 21, 2010.

(51) Int. Cl. A61K 38/07; C07K 5/103; A61K 45/06; C07K 5/107

(52) U.S. Cl. CPC A61K 38/07; C07K 5/1016; C07K 5/101; A61K 45/06
USPC 514/4.3; 530/330

(57) ABSTRACT

The present invention relates to macrocyclic compounds of formula I that are useful as inhibitors of the hepatitis C virus (HCV) NS3 protease, their synthesis, and their use for treating or preventing HCV infections.

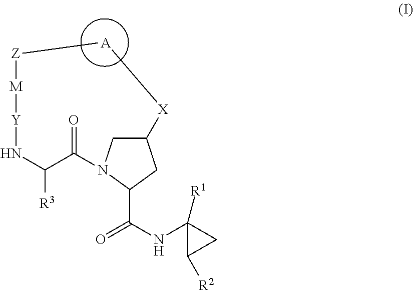

### FIELD OF THE INVENTION

The present invention relates to macrocyclic compounds that are useful as inhibitors of the hepatitis C virus (HCV) NS3 protease, the synthesis of such compounds, and the use of such compounds for treating HCV infection and/or reducing the likelihood or severity of symptoms of HCV infection.

### BACKGROUND OF THE INVENTION

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside analog ribavirin.

Several virally-encoded enzymes are putative targets for therapeutic intervention, including a metalloprotease (NS2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B).

Potential treatments for HCV infection has been discussed in the different references including Balsano, Mini Rev. Med. Chem. 8(4):307-318, 2008, Rönn et al., Current Topics in Medicinal Chemistry 8:533-562, 2008, Sheldon et al., Expert Opin. Investig. Drugs 16(8):1171-1181, 2007, and De Francesco et al., Antiviral Research 58:1-16, 2003.

There is a clear and long-felt need to develop effective therapeutics for treatment of HCV infection. Specifically, there is a need to develop compounds that are useful for treating HCV-infected patients and compounds that selectively inhibit HCV viral replication. Thus, there is a need for compounds that are effective inhibitors of the NS3 protease.

### SUMMARY OF THE INVENTION

The present invention relates to novel macrocyclic compounds of formula I and/or pharmaceutically acceptable salts or hydrates thereof. These compounds are useful in the inhibition of HCV (hepatitis C virus) NS3 (non-structural 3) protease, the prevention or treatment of one or more of the symptoms of HCV infection, either as compounds or their pharmaceutically acceptable salts or hydrates (when appropriate), or as pharmaceutical composition ingredients. As pharmaceutical composition ingredients, these compounds, salts and hydrates may be the primary active therapeutic agent, and, when appropriate, may be combined with other therapeutic agents including but not limited to other HCV antivirals, anti-infectives, immunomodulators, antibiotics or vaccines.

More particularly, the present invention relates to a compound of formula I and/or a pharmaceutically acceptable salt or hydrate thereof:

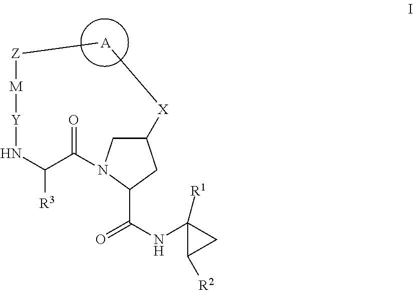

wherein:

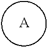

is one or more rings selected from the group consisting of:
a) aryl rings,
b) C₃-C₈ cycloalkyl rings, and
c) heterocyclic rings in which the heterocyclic ring system attaches to Z and X at points that are two independently selected ring atoms that are either two carbon ring atoms or one carbon ring atom and one nitrogen ring atom, and the heterocyclic ring system is selected from the group consisting of
   i) 5- or 6-membered saturated or unsaturated monocyclic rings with 1, 2, or 3 heteroatom ring atoms independently selected from the group consisting of N, O or S,
   ii) 8-, 9- or 10-membered saturated or unsaturated bicyclic rings with 1, 2, or 3 heteroatom ring atoms independently selected from the group consisting of N, O or S, and
   iii) 11- to 15-membered saturated or unsaturated tricyclic rings with 1, 2, 3, or 4 heteroatom ring atoms independently selected from the group consisting of N, O or S,
wherein

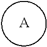

is substituted with 0 to 4 independently selected substituents R⁴, R⁵ or oxo; wherein for stable heterocyclic rings containing S or N, the heterocyclic ring is unsubstituted at the S or N atom or is substituted at the S by oxo; wherein the R⁴ and R⁵ substitutions are located on one or more ring atoms selected from C and N;

X is selected from the group consisting of —O—, —CH₂O—, —NHC(O)O—, —CH₂NHC(O)O—, —C≡CCH₂O—, —C(O)O—, —(CH₂)₃O—, —OC(O)NH—, —(CH₂)₂C(O)NH—, —C(O)NH— and a direct bond;

R¹ is selected from the group consisting of —CO₂R⁶, —CONR⁶SO₂R⁷, —CONR⁶SO₂NR⁸R⁹, tetrazolyl, —CONHP(O)R¹⁰R¹¹, and —P(O)R¹⁰R¹¹;

R² is selected from the group consisting of C₁-C₆ alkyl, C₂-C₆ alkenyl, and C₃-C₈ cycloalkyl, wherein the R² are substituted with 0 to 3 independently selected halogen atoms;

R³ is selected from the group consisting of H, C₁-C₈ alkyl, C₃-C₈ cycloalkyl, C₃-C₈ cycloalkyl(C₁-C₈)alkyl, phenyl(C₁-C₈)alkyl, naphthyl(C₁-C₈)alkyl, and Het groups, wherein when R³ is not H, the R³ is substituted with 0 to 3 substituents independently selected from the group consisting of halogen atoms, —OR⁶, —SR⁶, —N(R⁶)₂, —N(C₁-C₆ alkyl)O(C₁-C₆ alkyl), C₁-C₆ alkyl, C₁-C₆ haloalkyl, halo(C₁-C₆ alkoxy), —NO₂, —CN, —CF₃, —SO₂(C₁-C₆ alkyl), —S(O)(C₁-C₆ alkyl), —NR⁶SO₂R⁷, SO₂N(R⁷)₂, —NHCOOR⁷, —NHCOR⁷, —NHCONHR⁷, —CO₂R⁶, —C(O)R⁶, and —CON(R⁶)₂;

Het is selected from the group consisting of substituted and unsubstituted 5- and 6-membered saturated heterocyclic rings having 1 or 2 heteroatoms independently selected from N, O and S;

Y is selected from the group consisting of —C(O)—, —SO₂—, —OC(O)—, —C(O)N(R¹²)L- and -LN(R¹²)C(O)—, where
R¹² is selected from the group consisting of H, C₁-C₆ alkyl and C₁-C₆ alkenyl groups,
L is selected from the group consisting of a direct bond, -G-(C₁-C₆ alkylene)-, —(C₁-C₆ alkylene)-G-, -G-(C₁-C₆ alkenylene)-, and —(C₁-C₆ alkenylene)-G-, groups, where the G is selected from the group consisting of a direct bond, —O—, —N— and —S—, the alkylene and alkenylene groups are substituted with 0 to 4 substituents R¹³ independently selected from the group consisting of C₁-C₆ alkyl and C₁-C₆ alkenyl groups, and
the R¹² and R¹³ may be taken together to form a 3- to 6-membered ring containing 0 to 3 heteroatoms selected from N, O and S;

M is selected from the group consisting of C₁-C₁₂ alkylenes, C₂-C₁₂ alkenylenes and C₂-C₁₂ alkynylenes, wherein:
the M contains 0 or 1-O— moiety in place of a methylene moiety, and
the M is substituted with from 0 to 4 substituents R¹⁴, wherein:
   each R¹⁴ is independently selected from the group consisting of C₁-C₈ alkyl, ═CH₂, C₃-C₈ cycloalkyl(C₁-C₈ alkyl), and aryl(C₁-C₈ alkyl), and
   any substituent R¹⁴ may be taken together with any adjacent substituent R¹⁴ or any adjacent substituent R¹² or R¹³ to form a 3- to 6-membered ring containing 0 to 3 heteroatoms independently selected from the group consisting of N, O and S;

Z is selected from the group consisting of —C(O)— and a direct bond;

R⁴ is selected from the group consisting of H, halogen atoms, —OH, C₁-C₆ alkoxy, C₁-C₆ alkyl, —CN, —CF₃, —OCF₃, —C(O)OH, —C(O)CH₃, —SR⁶, —SO₂(C₁-C₆ alkyl), C₃-C₈ cycloalkyl, C₃-C₈ cycloalkoxy, C₁-C₆ haloalkyl, —N(R¹⁵)₂, phenyl, naphthyl, —O-phenyl, —O-naphthyl, heteroaryl and heterocyclyl groups; wherein:
the R⁴ heteroaryl is selected from the group consisting of 5- and 6-membered aromatic rings having 1, 2 or 3 heteroatoms independently selected from N, O and S, and the R⁴ heteroaryl is attached through a ring atom selected from C or N,
the R⁴ heterocyclyl is selected from the group consisting of 5- to 7-membered saturated or unsaturated non-aromatic rings having 1, 2, 3 or 4 heteroatoms independently selected from N, O and S, and the R⁴ heterocyclyl is attached through a ring atom selected from C or N, and
the R⁴ heteroaryl, heterocyclyl, cycloalkyl, cycloalkoxy, alkyl and alkoxy groups are substituted with 0 to 4 substituents independently selected from the group consisting of halogen atoms, —OR⁶, —SR⁶, —N(R⁶)₂, —N(C₁-C₆ alkyl)O(C₁-C₆ alkyl), C₁-C₆ alkyl, C₁-C₆ haloalkyl, halo(C₁-C₆ alkoxy), —NO₂, —CN, —CF₃, —SO₂(C₁-C₆ alkyl), —S(O)(C₁-C₆ alkyl), —NR⁶SO₂R⁷, SO₂N(R⁷)₂, —NHCOOR⁷, —NHCOR⁷, —NHCONHR⁷, —CO₂R⁶, —C(O)R⁶, and —CON(R⁶)₂, and 2 adjacent substituents of the R⁴ heteroaryl, heterocyclyl, cycloalkyl, cycloalkoxy, alkyl and alkoxy groups may be taken together to form a 3- to 6-membered cyclic ring containing 0 to 3 heteroatoms independently selected from N, O and S;

each R⁵ is independently selected from the group consisting of H, halogen atoms, —OR⁶, C₁-C₆ alkyl, —CN, —CF₃, —NO₂, —SR⁶, —CO₂R⁶, —CON(R⁶)₂, —C(O)R⁶, —N(R⁶)C(O)R⁶, —SO₂(C₁-C₆ alkyl), —S(O)(C₁-C₆ alkyl), C₃-C₈cycloalkyl, C₃-C₈cycloalkoxy, C₁-C₆ haloalkyl, —N(R⁶)₂, —N(C₁-C₆ alkyl)O(C₁-C₆ alkyl), halo(C₁-C₆ alkoxy), —NR⁶SO₂R⁶, —SO₂N(R⁶)₂, —NHCOOR⁶, —NHCONHR⁶, phenyl, naphthyl, heteroaryl and heterocyclyl groups, wherein
the R⁵ heteroaryl is selected from the group consisting of 5- and 6-membered aromatic rings having 1, 2 or 3 heteroatoms independently selected from N, O and S, and the R⁵ heteroaryl is attached through a ring atom selected from C or N,
the R⁵ heterocyclyl is selected from the group consisting of 5- to 7-membered saturated or unsaturated non-aromatic rings having 1, 2, 3 or 4 heteroatoms independently selected from N, O and S, and the R⁵ heterocyclyl is attached through a ring atom selected from C or N;

each R⁶ is independently selected from the group consisting of H, C₁-C₆ alkyl and benzyl, wherein each R⁶ is independently substituted with 0 to 4 substituents selected from the group consisting of halogen atoms, C₁-C₆ alkyl, C₁-C₆ haloalkyl, phenyl, naphthyl, C₃-C₈ cycloalkyl, heteroaryl, heterocyclyl, halo(C₁-C₆ alkoxy), —OH, —O(C₁-C₆ alkyl), —SH, —S(C₁-C₆ alkyl), —NH₂, —NH(C₁-C₆ alkyl), —N(C₁-C₆ alkyl)₂, —C(O)(C₁-C₆ alkyl), NO₂, —CN, —CF₃, —SO₂(C₁-C₆ alkyl), —S(O)(C₁-C₆ alkyl), —N(C₁-C₆ alkyl)SO₂(C₁-C₆ alkyl), —SO₂N(C₁-C₆ alkyl)₂, —NHCOO(C₁-C₆ alkyl), —NHCO(C₁-C₆ alkyl), —NHCONH(C₁-C₆ alkyl), —CO₂(C₁-C₆ alkyl), and —C(O)N(C₁-C₆ alkyl)₂;

R⁷ is selected from the group consisting of H, C₁-C₆ alkyl, C₃-C₆ cycloalkyl, C₃-C₆ cycloalkyl(C₁-C₅)alkyl, phenyl, naphthyl, phenyl(C₁-C₄)alkyl, naphthyl(C₁-C₄)alkyl, heteroaryl, heteroaryl(C₁-C₄ alkyl), heterocyclyl, and heterocyclyl(C₁-C₈ alkyl) groups, wherein
the R⁷ are substituted with 0 to 2 independently selected R⁵ substituents,
each R⁷ heteroaryl is independently selected from the group consisting of 5- and 6-membered aromatic rings having 1, 2 or 3 heteroatoms independently selected from N, O and S, and the R⁷ heteroaryl is attached through a ring atom selected from C or N, and
each R⁷ heterocyclyl is independently selected from the group consisting of 5- to 7-membered saturated or unsaturated non-aromatic rings having 1, 2, 3 or 4 heteroatoms independently selected from N, O and S, and the R⁷ heterocyclyl is attached through a ring atom selected from C or N;

R⁸ is selected from the group consisting of H, C₁-C₈ alkyl, C₃-C₈ cycloalkyl, C₃-C₈ cycloalkyl(C₁-C₈ alkyl), phenyl, naphthyl, phenyl(C₁-C₄)alkyl, naphthyl(C₁-C₄)alkyl, heteroaryl, heterocyclic, heteroaryl(C₁-C₄ alkyl), and heterocyclyl(C₁-C₈ alkyl) groups, wherein
the R⁸ are substituted with 0 to 4 substituents selected from the group consisting of phenyl, naphthyl, C₃-C₈ cycloalkyl, heteroaryl, heterocyclyl, C₁-C₆ alkyl, halo(C₁-C₆ alkoxy), halogen atoms, —OR⁶, —SR⁶, —N(R⁶)₂, —N(C₁-C₆ alkyl)O(C₁-C₆ alkyl), C₁-C₆ alkyl, —C(O)R⁶, C₁-C₆ haloalkyl, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂,
each R⁸ heteroaryl is independently selected from the group consisting of 5- and 6-membered aromatic rings having 1, 2 or 3 heteroatoms independently selected from N, O and S, and the R⁸ heteroaryl is attached through a ring atom selected from C or N,
each R⁸ heterocyclyl is independently selected from the group consisting of 5- to 7-membered saturated or unsaturated non-aromatic rings having 1, 2, 3 or 4 heteroatoms independently selected from N, O and S, and the R⁸ heterocyclyl is attached through a ring atom selected from C or N, and
2 adjacent substituents of the R⁸ may be taken together to form a 3- to 6-membered ring containing 0 to 3 heteroatoms independently selected from the group consisting of N, O and S;

R⁹ is selected from the group consisting of H, C₁-C₈ alkyl, C₃-C₈ cycloalkyl, C₃-C₈ cycloalkyl(C₁-C₈ alkyl), C₁-C₈ alkoxy, C₃-C₈ cycloalkoxy, phenyl, naphthyl, phenyl(C₁-C₄)alkyl, naphthyl(C₁-C₄)alkyl, heteroaryl, heterocyclyl, heteroaryl(C₁-C₄ alkyl), or heterocyclyl(C₁-C₈ alkyl) groups, wherein
the R⁹ are substituted with 0 to 4 substituents selected from the group consisting of phenyl, naphthyl, C₃-C₈ cycloalkyl, heteroaryl, heterocyclyl, C₁-C₆ alkyl, halo(C₁-C₆ alkoxy), halogen atoms, —OR⁶, —SR⁶, —N(R⁶)₂, —N(C₁-C₆ alkyl)O(C₁-C₆ alkyl), C₁-C₆ alkyl, —C(O)R⁶, C₁-C₆ haloalkyl, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂,
each R⁹ heteroaryl is independently selected from the group consisting of 5- and 6-membered aromatic rings having 1, 2 or 3 heteroatoms independently selected from N, O and S, and the R⁹ heteroaryl is attached through a ring atom selected from C or N,
each R⁹ heterocyclyl is independently selected from the group consisting of 5- to 7-membered saturated or unsaturated non-aromatic rings having 1, 2, 3 or 4 heteroatoms independently selected from N, O and S, and the R⁹ heterocyclyl is attached through a ring atom selected from C or N, and
2 adjacent substituents of the R⁹ may be taken together to form a 3- to 6-membered ring containing 0 to 3 heteroatoms independently selected from the group consisting of N, O and S, and

R⁸ and R⁹ may be taken together, with the N to which they are attached, to form a 4- to 8-membered monocyclic ring containing 0 to 2 additional heteroatoms independently selected from N, O and S;

each R¹⁰ is independently selected from the group consisting of H, C₁-C₆ alkyl, C₁-C₄ alkenyl, —OR¹⁶, —N(R⁶)—V—CO₂R⁶, —O—V—CO₂R⁶, —S—V—CO₂R⁶, —N(R⁶)(R¹⁶), —R¹⁷, and —N(R⁶)SO₂R⁷;

each R¹¹ is independently selected from the group consisting of H, —OR¹⁶, —N(R⁶)—V—CO₂R⁶, —O—V—CO₂R⁶, —S—V—CO₂R⁶, and —N(R⁶)(R⁶);

R¹⁰ and R¹¹ may be taken together, with the phosphorus atom to which they are attached, to form a 5- to 7-membered monocyclic ring;

each V is independently selected from the group consisting of —CH(R¹⁸)— and —(C₁-C₄ alkylene)-CH(R¹⁸)—;

each R¹⁵ is independently selected from the group consisting of H, C₁-C₆ alkyl, C₃-C₆ cycloalkyl, C₃-C₆ cycloalkyl(C₁-C₅)alkyl, phenyl, naphthyl, phenyl(C₁-C₄)alkyl, naphthyl(C₁-C₄)alkyl, heteroaryl, heteroaryl(C₁-C₄ alkyl), heterocyclyl, and heterocyclyl(C₁-C₈ alkyl) groups, wherein
when R¹⁵ is not H, the R¹⁵ are substituted with 0 to 2 R⁵ substituents,
each R¹⁵ heteroaryl is independently selected from the group consisting of 5- and 6-membered aromatic rings having 1, 2 or 3 heteroatoms independently selected from N, O and S, and the R¹⁵ heteroaryl is attached through a ring atom selected from C or N, and
each R¹⁵ heterocyclyl is independently selected from the group consisting of 5- to 7-membered saturated or unsaturated non-aromatic rings having 1, 2, 3 or 4 heteroatoms independently selected from N, O and S, and the R¹⁵ heterocyclyl is attached through a ring atom selected from C or N, and
the R¹⁵ may be taken together with the atom to which it is attached and a second R¹⁵ substituent to form a 4- to 7-membered heterocyclic ring;

each R¹⁶ is independently selected from the group consisting of H, C₁-C₆ alkyl, C₂-C₆ alkenyl, C₃-C₈ cycloalkyl, aryl, heteroaryl, and heterocyclyl groups, wherein
when R¹⁶ is not H, the R¹⁶ is substituted with 0 to 2 substituents independently selected from the group consisting of phenyl, naphthyl, phenyl(C₁-C₄ alkyl), naphthyl(C₁-C₄ alkyl), C₃-C₈ cycloalkyl, C₃-C₈ cycloalkyl(C₁-C₄ alkyl), heteroaryl, heteroaryl(C₁-C₄ alkyl), heterocyclyl, heterocyclyl(C₁-C₄ alkyl), C₁-C₆ alkyl, halogen atoms, —OC(O)OR⁷, —OC(O)R⁷, —OR⁶, —SR⁶, —N(R⁶)₂, —C(O)R⁶, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂,
each R¹⁶ heteroaryl is independently selected from the group consisting of 5- and 6-membered aromatic rings having 1, 2 or 3 heteroatoms independently selected from N, O and S, and the R¹⁶ heteroaryl is attached through a ring atom selected from C or N,
each R¹⁶ heterocyclyl is independently selected from the group consisting of 5- to 7-membered saturated or unsaturated non-aromatic rings having 1, 2, 3 or 4 heteroatoms independently selected from N, O and S, and the R¹⁶ heterocyclyl is attached through a ring atom selected from C or N, and
2 adjacent substituents of the R¹⁶ may be taken together to form a 3- to 6-membered ring containing 0 to 3 heteroatoms independently selected from the group consisting of N, O and S;

R¹⁷ is selected from the group consisting of H, C₁-C₆ alkyl, C₂-C₆ alkenyl, phenyl, naphthyl and heteroaryl, wherein
each R¹⁷ heteroaryl is independently selected from the group consisting of 5- and 6-membered aromatic rings having 1, 2 or 3 heteroatoms independently selected from N, O and S, and the R¹⁷ heteroaryl is attached through a ring atom selected from C or N, and
the R¹⁷ phenyl, naphthyl or heteroaryl may be substituted with 0 to 2 substituents independently selected from the group consisting of C₁-C₆ alkyl, halogen atoms, —OC(O)OR⁷, —OC(O)R⁷, —OR⁶, —SR⁶, —N(R⁶)₂, —C(O)R⁶, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂; and

each R¹⁸ is independently selected from the group consisting of H, C₁-C₆ alkyl, C₂-C₆ alkenyl, C₃-C₈ cycloalkyl, phenyl, naphthyl, heteroaryl, and heterocyclyl groups, wherein
the R¹⁸ are substituted with 0 to 2 substituents independently selected from the group consisting of C₁-C₆ alkyl, halogen atoms, —OC(O)OR⁷, —OC(O)R⁷, —OR⁶, —SR⁶, —N(R⁶)₂, —C(O)R⁶, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂,
each R¹⁸ heteroaryl is independently selected from the group consisting of 5- and 6-membered aromatic rings having 1, 2 or 3 heteroatoms independently selected from N, O and S, and the R¹⁸ heteroaryl is attached through a ring atom selected from C or N,
each R¹⁸ heterocyclyl is independently selected from the group consisting of 5- to 7-membered saturated or unsaturated non-aromatic rings having 1, 2, 3 or 4 heteroatoms independently selected from N, O and S, and the R¹⁸ heterocyclyl is attached through a ring atom selected from C or N, and
2 adjacent substituents of the R¹⁸ are optionally taken together to form a 3- to 6-membered ring containing 0 to 3 heteroatoms independently selected from the group consisting of N, O and S.

The present invention also includes pharmaceutical compositions containing a compound of the present invention and methods of preparing such pharmaceutical compositions. The present invention further includes methods of treating or preventing one or more symptoms of HCV infection.

Other embodiments, aspects and features of the present invention are either further described in or will be apparent from the ensuing description, examples and appended claims.

### DETAILED DESCRIPTION OF THE INVENTION

The present invention includes compounds of formula I above, and pharmaceutically acceptable salts and/or hydrates thereof. These compounds and their pharmaceutically acceptable salts and/or hydrates are HCV protease inhibitors (e.g., HCV NS3 protease inhibitors).

In a first embodiment of the invention,

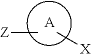

is selected from the group consisting of:

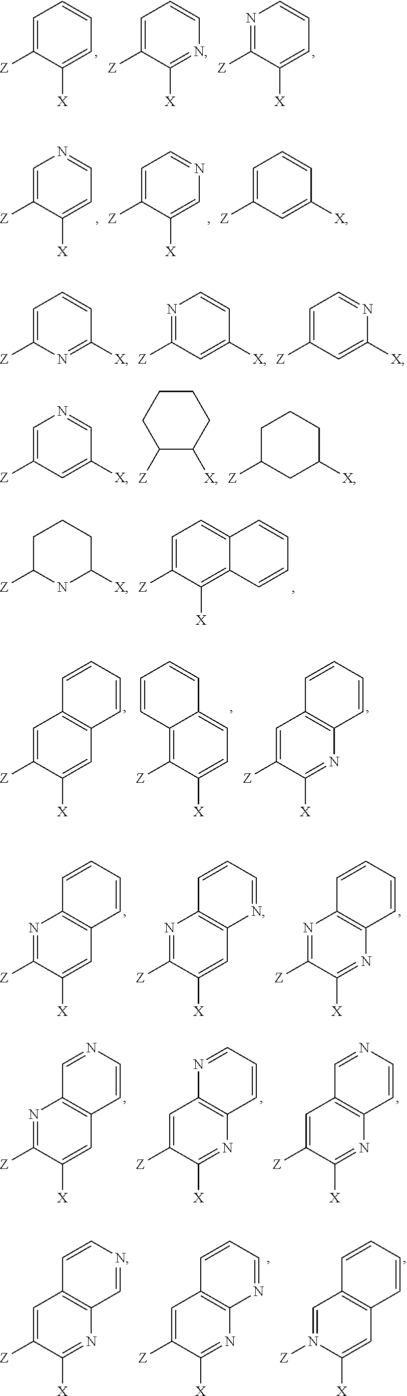

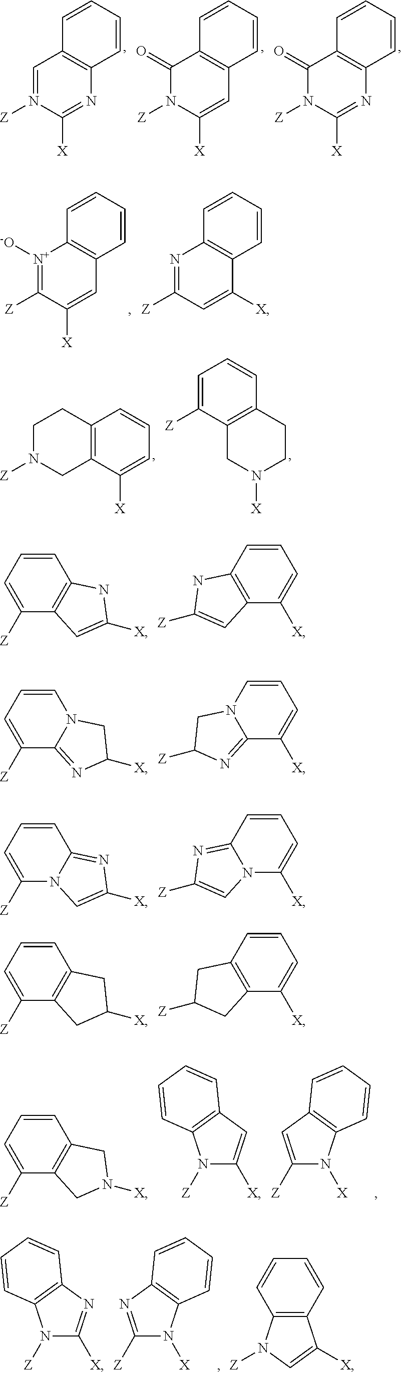

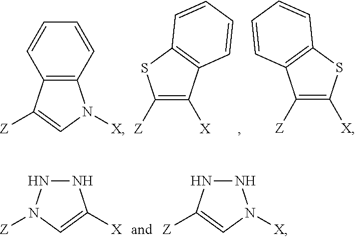

wherein said

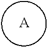

is substituted with 0 to 4 independently selected substituents R⁴, R⁵ or oxo; wherein for stable heterocyclic rings containing S or N, the heterocyclic ring is unsubstituted at the S or N atom or is substituted at the S or N atom by oxo; wherein said R⁴ and R⁵ substitutions are located on one or more ring atoms selected from C and N. In aspects of the first embodiment,

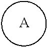

is unsubstituted or mono-substituted with a moiety selected from the group consisting of —Br, —Cl, —CN, phenyl, —O-phenyl, —OCF₃, —OCH₃, —OH, C₁-C₆ alkoxy, C₁-C₆ alkyl, —CF₃, —C(O)OH, and C(O)CH₃. In particular aspects of the first embodiment,

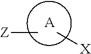

is selected from the group consisting of

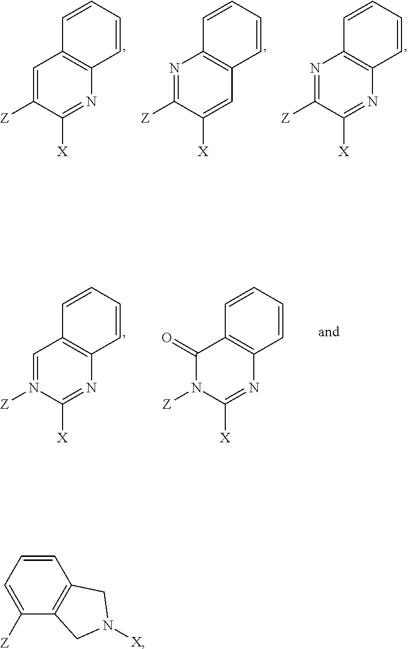

and substituted with 0 to 2 independently selected substituents —Cl, —O-benzyl, —OCH₃, —OH, —CH₃. In all aspects of this embodiment, all other groups are as provided in the general formula above.

In a second embodiment of the invention, X is selected from the group consisting of —O— and —C(O)O—. In this embodiment, all other groups are as provided in the general formula above or in the first embodiment.

In a third embodiment of the invention, R¹ is selected from the group consisting of —CO₂R⁶ and —CONR⁶SO₂R⁷. In aspects of this embodiment, R¹ is selected from the group consisting of

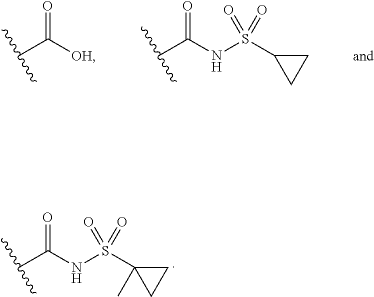

In particular aspects of this embodiment, R¹ is selected from the group consisting of

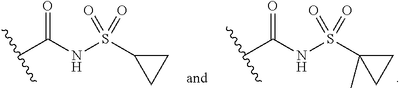 [SMILES: *C(=O)NS(=O)(=O)C1CC1 ; *C(=O)NS(=O)(=O)C1(C)CC1]

In all aspects of this embodiment, all other groups are as provided in the general formula above or in either or both of the first or second embodiments.

In a fourth embodiment of the invention, R² is selected from the group consisting of C₁-C₆ alkyl and C₂-C₆ alkenyl. In aspects of this embodiment, R² is selected from the group consisting of —CH═CH₂, —CH₂CH₃, and —CH₂CH═CH₂. In particular aspects of this embodiment, R² is selected from the group consisting of —CH═CH₂ and —CH₂CH₃. In all aspects of this embodiment, all other groups are as provided in the general formula above or in any or all of the first through third embodiments.

In a fifth embodiment of the invention, R³ is selected from the group consisting of H, C₁-C₈ alkyl and C₃-C₈ cycloalkyl. In aspects of this embodiment, R³ is selected from the group consisting of H, —C(CH₃)₃, —(CH₂)₃CH₃, cyclohexyl, and —CH(CH₃)₂. In particular aspects of this embodiment, R³ is selected from the group consisting of —C(CH₃)₃ and cyclohexyl. In all aspects of this embodiment, all other groups are as provided in the general formula above or in any or all of the first through fourth embodiments.

In a sixth embodiment of the invention, Y is selected from the group consisting of —OC(O)—, —C(O)N(R¹²)L- and -LN(R¹²)C(O)—. In aspects of this embodiment, Y is —OC(O)—. In this embodiment, all other groups are as provided in the general formula above or in any or all of the first through fifth embodiments.

In a seventh embodiment of the invention, M is selected from the group consisting of C₁-C₁₂ alkylene or C₂-C₁₂ alkenylene, wherein M is substituted with 0 to 3 substituents R¹⁴ selected from the group consisting of C₁-C₈ alkyl, and ═CH₂. In aspects of this embodiment, Z-M-Y is selected from the group consisting of

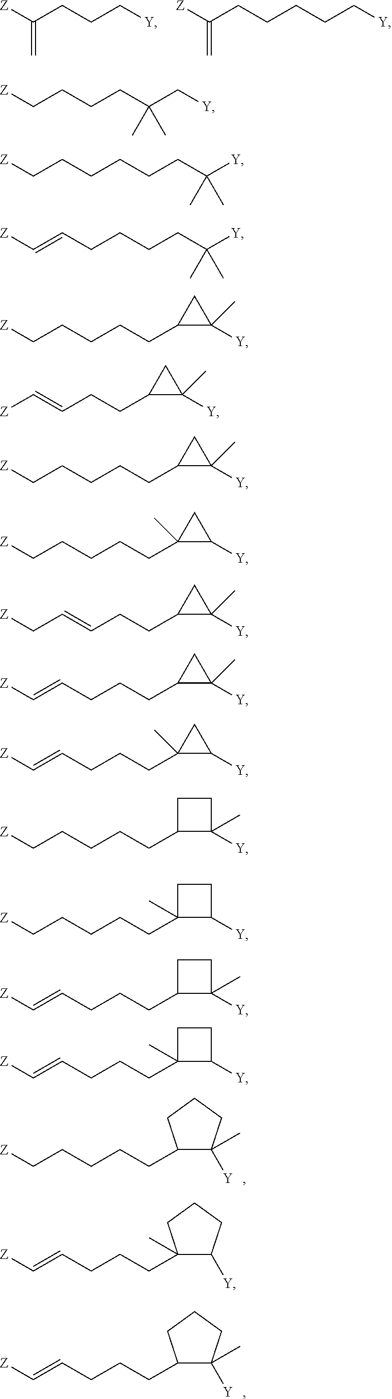

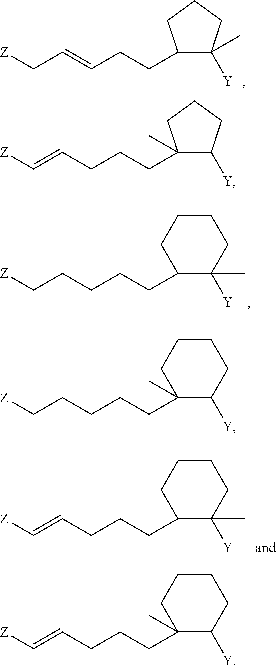

In still further aspects of this embodiment, Z-M-Y is selected from the group consisting of

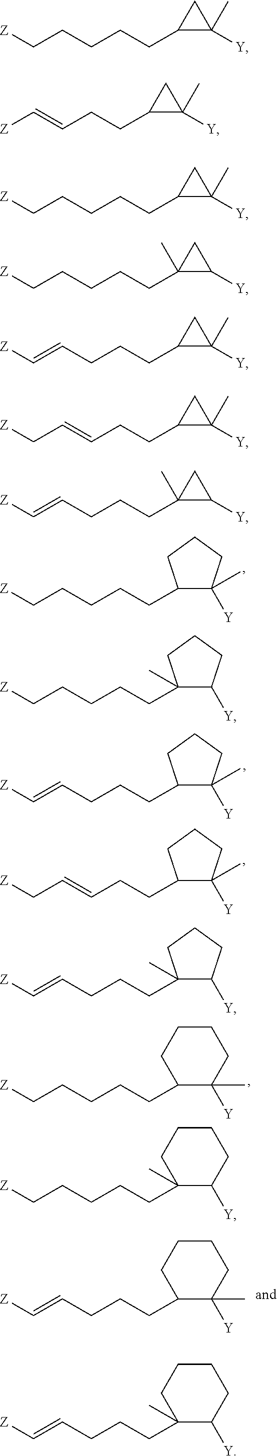

In particular aspects of this embodiment, Z-M-Y is selected from the group consisting of

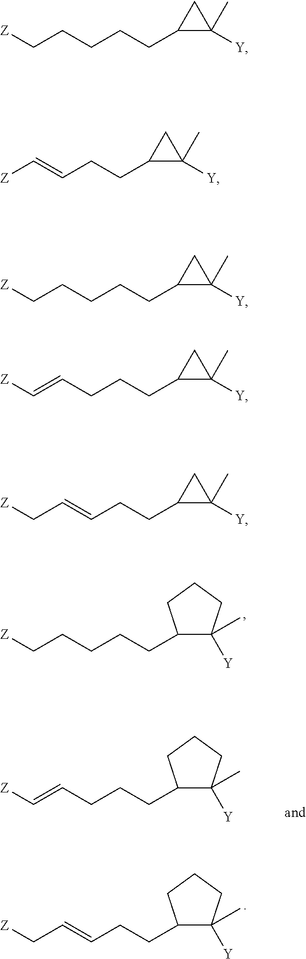

In all aspects of this embodiment, all other groups are as provided in the general formula above or in any or all of the first through sixth embodiments.

In an eighth embodiment of the invention, one or more substituents R¹⁴ are taken together and/or with one or more substituents chosen from substituents R¹² and R¹³ to form a 3- to 6-membered ring containing 0 to 3 heteroatoms selected from the group consisting of N, O and S. In this embodiment, all other groups are as provided in the general formula above or in any or all of the first through seventh embodiments.

In a ninth embodiment of the invention, Z is a direct bond. In this embodiment, all other groups are as provided in the general formula above or in any or all of the first through eighth embodiments.

In a tenth embodiment of the invention,

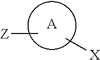

is selected from the group consisting of:

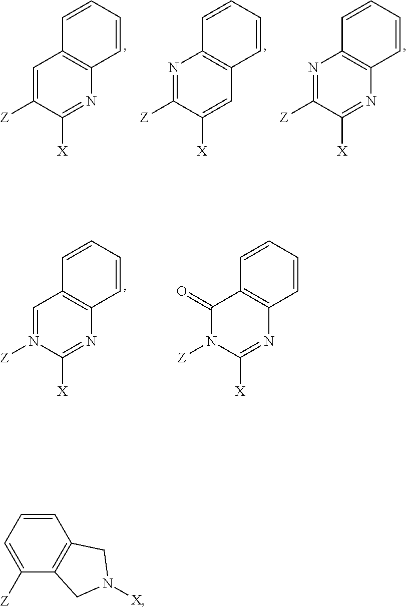

and wherein said

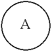

is substituted with 0 to 2 independently selected substituents —Cl, —O-benzyl, —OCH₃, —OH, —CH₃; X is selected from the group consisting of —O— and —C(O)O—; R¹ is selected from the group consisting of

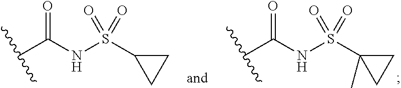 [SMILES: *C(=O)NS(=O)(=O)C1CC1 ; *C(=O)NS(=O)(=O)C1(C)CC1]

R² is selected from the group consisting of —CH═CH₂ and —CH₂CH₃; R³ is selected from the group consisting of —C(CH₃)₃ and cyclohexyl; Y is —OC(O)—; Z-M-Y is selected from the group consisting of

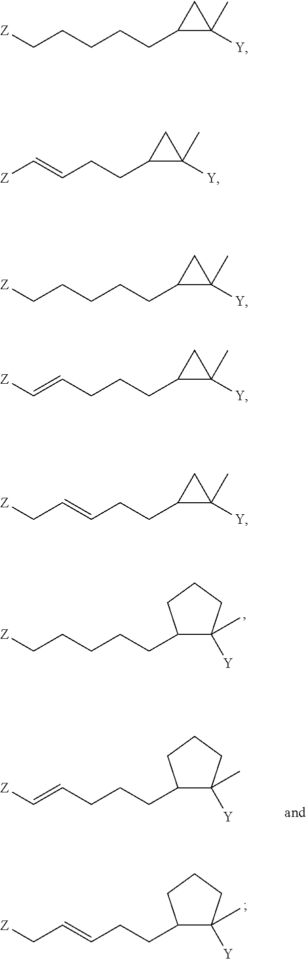

and Z is a direct bond.

In a tenth embodiment of the invention, the compound is selected from the compounds of Examples 1 through 32, i.e. compounds I-1 through I-32 and pharmaceutically acceptable salts thereof.

In another embodiment of the invention, the compound of the invention is selected from the exemplary species depicted in Examples 1 through 32 shown below.

Other embodiments of the present invention include the following:

(a) A pharmaceutical composition comprising an effective amount of a compound of formula I and a pharmaceutically acceptable carrier.

(b) The pharmaceutical composition of (a), further comprising a second therapeutic agent selected from the group consisting of HCV antiviral agents, immunomodulators, and anti-infective agents.

(c) The pharmaceutical composition of (b), wherein the HCV antiviral agent is an antiviral selected from the group consisting of HCV protease inhibitors and HCV NS5B polymerase inhibitors.

(d) A pharmaceutical combination which is (i) a compound of formula I and (ii) a second therapeutic agent selected from the group consisting of HCV antiviral agents, immunomodulators, and anti-infective agents; wherein the compound of formula I and the second therapeutic agent are each employed in an amount that renders the combination effective for inhibiting HCV NS3 protease, or for treating HCV infection and/or reducing the likelihood or severity of symptoms of HCV infection.

(e) The combination of (d), wherein the HCV antiviral agent is an antiviral selected from the group consisting of HCV protease inhibitors and HCV NS5B polymerase inhibitors.

(f) A method of inhibiting HCV NS3 protease in a subject in need thereof which comprises administering to the subject an effective amount of a compound of formula I.

(g) A method of treating HCV infection and/or reducing the likelihood or severity of symptoms of HCV infection in a subject in need thereof which comprises administering to the subject an effective amount of a compound of formula I.

(h) The method of (g), wherein the compound of formula I is administered in combination with an effective amount of at least one second therapeutic agent selected from the group consisting of HCV antiviral agents, immunomodulators, and anti-infective agents.

(i) The method of (h), wherein the HCV antiviral agent is an antiviral selected from the group consisting of HCV protease inhibitors and HCV NS5B polymerase inhibitors.

(j) A method of inhibiting HCV NS3 protease in a subject in need thereof which comprises administering to the subject the pharmaceutical composition of (a), (b), or (c) or the combination of (d) or (e).

(k) A method of treating HCV infection and/or reducing the likelihood or severity of symptoms of HCV infection in a subject in need thereof which comprises administering to the subject the pharmaceutical composition of (a), (b), or (c) or the combination of (d) or (e).

The present invention also includes a compound of the present invention for use (i) in medicine, (ii) as a medicament for, or (iii) in the preparation of a medicament for:

(a) inhibiting HCV NS3 protease, or (b) treating HCV infection and/or reducing the likelihood or severity of symptoms of HCV infection. In these uses, the compounds of the present invention can optionally be employed in combination with one or more second therapeutic agents selected from HCV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

In the embodiments of the compound provided above, it is to be understood that each embodiment may be combined with one or more other embodiments, to the extent that such a combination provides a stable compound and is consistent with the description of the embodiments. It is further to be understood that the embodiments of compositions and methods provided as (a) through (k) above are understood to include all embodiments of the compounds, including such embodiments as result from combinations of embodiments.

As used herein, all ranges are inclusive, and all sub-ranges are included within such ranges, although not necessarily explicitly set forth. In addition, the term “or,” as used herein, denotes alternatives that may, where appropriate, be combined; that is, the term “or” includes each listed alternative separately as well as their combination.

As used herein, the term “alkyl” refers to any linear or branched chain alkyl group having a number of carbon atoms in the specified range. Thus, for example, “C₁₋₆alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl. The term “alkoxy” refers to an “alkyl-O—” group.

The term “halogenated” refers to a group or molecule in which a hydrogen atom has been replaced by a halogen. Similarly, the term “haloalkyl” refers to a halogenated alkyl group. The term “halogen” (or “halo”) refers to atoms of fluorine, chlorine, bromine and iodine (alternatively referred to as fluoro, chloro, bromo, and iodo).

The term “alkylene” refers to any linear or branched chain alkylene group (or alternatively “alkanediyl”) having a number of carbon atoms in the specified range. Thus, for example, “—C₁₋₆alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Also of interest is the alkylene —CH(CH₃)—.

The terms “cycloalkyl” refers to any cyclic ring of an alkane or alkene having a number of carbon atoms in the specified range. Thus, for example, “C₃₋₈cycloalkyl” (or “C₃-C₈ cycloalkyl”) refers to cyclopropyl, cyclobutyl, cyclopentyl, cyclohexyl, cycloheptyl, and cyclooctyl. The term “cycloalkoxy” refers to a “—O-cycloalkyl” group.

The term “Het” refers to a 5- to 6-membered saturated cyclic ring having 1 or 2 heteroatoms selected from N, O and S, wherein said ring is optionally substituted with 1 to 3 substituents selected from halogen atoms, —OR⁶, —SR⁶, —N(R⁶)₂, —N(C₁-C₆ alkyl)O(C₁-C₆ alkyl), C₁-C₆ alkyl, C₁-C₆ haloalkyl, halo(C₁-C₆ alkoxy), —NO₂, —CN, —CF₃, —SO₂(C₁-C₆ alkyl), —S(O)(C₁-C₆ alkyl), —NR⁶SO₂R⁷, SO₂N(R⁷)₂, —NHCOOR⁷, —NHCOR⁷, —NHCONHR⁷, —CO₂R⁶, —C(O)R⁶, and —CON(R⁶)₂.

The term “carbocycle” (and variations thereof such as “carbocyclic” or “carbocyclyl”) as used herein, unless otherwise indicated, refers to (i) a C₃ to C₈ monocyclic, saturated or unsaturated ring or (ii) a C₇ to C₁₂ bicyclic saturated or unsaturated ring system. Each ring in (ii) is either independent of, or fused to, the other ring, and each ring is saturated or unsaturated. The carbocycle may be attached to the rest of the molecule at any carbon atom which results in a stable compound. The fused bicyclic carbocycles are a subset of the carbocycles; i.e., the term “fused bicyclic carbocycle” generally refers to a C₇ to C₁₀ bicyclic ring system in which each ring is saturated or unsaturated and two adjacent carbon atoms are shared by each of the rings in the ring system. A fused bicyclic carbocycle in which one ring is saturated and the other is saturated is a saturated bicyclic ring system. A fused bicyclic carbocycle in which one ring is benzene and the other is saturated is an unsaturated bicyclic ring system. A fused bicyclic carbocycle in which one ring is benzene and the other is unsaturated is an unsaturated ring system. Saturated carbocyclic rings are also referred to as cycloalkyl rings, e.g., cyclopropyl, cyclobutyl, etc. Unless otherwise noted, carbocycle is unsubstituted or substituted with C₁₋₆alkyl, C₁₋₆alkenyl, C₁₋₆alkynyl, aryl, halogen, NH₂ or OH. A subset of the fused bicyclic unsaturated carbocycles are those bicyclic carbocycles in which one ring is a benzene ring and the other ring is saturated or unsaturated, with attachment via any carbon atom that results in a stable compound. Representative examples of this subset include

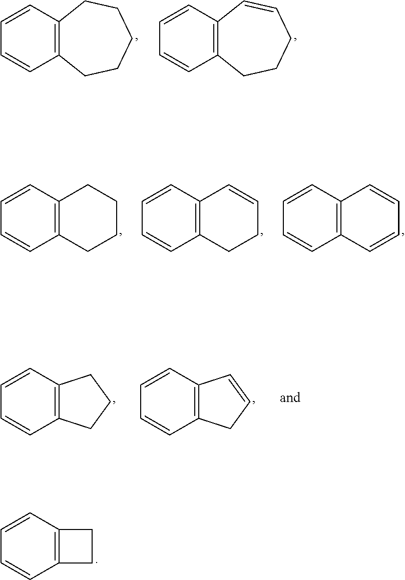

Depicted ring systems include, where appropriate, an indication of the variable to which a particular ring atom is attached. For example, the indole structure

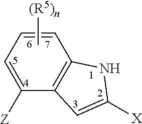

shows ring atom 2 is directly attached to variable X and ring atom 4 is directly attached to variable Z. Variable R⁵ is shown as a floating variable which can be attached to any ring atom, provided that such attachment results in formation of a stable ring.

The term “aryl” refers to aromatic mono- and poly-carbocyclic ring systems, also referred to as “arenes”, wherein the individual carbocyclic rings in the polyring systems are fused or attached to each other via a single bond. Suitable aryl groups include phenyl, naphthyl, and biphenylenyl.

Unless indicated otherwise, the term “heterocycle” (and variations thereof such as “heterocyclic” or “heterocyclyl”) broadly refers to (i) a stable 4- to 8-membered, saturated or unsaturated monocyclic ring, (ii) a stable 7- to 12-membered bicyclic ring system, or (iii) a stable 11- to 15-membered tricyclic ring system, wherein each ring in (ii) and (iii) is independent of, or fused to, the other ring or rings and each ring is saturated or unsaturated, and the monocyclic ring, bicyclic ring system or tricyclic ring system contains one or more heteroatoms (e.g., from 1 to 6 heteroatoms, or from 1 to 4 heteroatoms) selected from N, O and S and a balance of carbon atoms (the monocyclic ring typically contains at least one carbon atom and the bicyclic and tricyclic ring systems typically contain at least two carbon atoms); and wherein any one or more of the nitrogen and sulfur heteroatoms is optionally oxidized, and any one or more of the nitrogen heteroatoms is optionally quaternized. Unless otherwise specified, the heterocyclic ring may be attached at any heteroatom or carbon atom, provided that attachment results in the creation of a stable structure. Unless otherwise specified, when the heterocyclic ring has substituents, it is understood that the substituents may be attached to any atom in the ring, whether a heteroatom or a carbon atom, provided that a stable chemical structure results.

Saturated heterocyclics form a subset of the heterocycles. Unless expressly stated to the contrary, the term “saturated heterocyclic” generally refers to a heterocycle as defined above in which the entire ring system (whether mono- or poly-cyclic) is saturated. The term “saturated heterocyclic ring” refers to a 4- to 8-membered saturated monocyclic ring, a stable 7- to 12-membered bicyclic ring system, or a stable 11- to 15-membered tricyclic ring system, which consists of carbon atoms and one or more heteroatoms selected from N, O and S. Representative examples include piperidinyl, piperazinyl, azepanyl, pyrrolidinyl, pyrazolidinyl, imidazolidinyl, oxazolidinyl, isoxazolidinyl, morpholinyl, thiomorpholinyl, thiazolidinyl, isothiazolidinyl, and tetrahydrofuryl (or tetrahydrofuranyl).

Unsaturated heterocyclics form another subset of the heterocycles. Unless expressly stated to the contrary, the term “unsaturated heterocyclic” generally refers to a heterocycle as defined above in which the entire ring system (whether mono- or poly-cyclic) is not saturated, i.e., such rings are either unsaturated or partially unsaturated. Unless expressly stated to the contrary, the term “heteroaromatic ring” refers to a 5- or 6-membered monocyclic aromatic ring, a 7- to 12-membered bicyclic ring system, or an 11- to 15-membered tricyclic ring system, which consists of carbon atoms and one or more heteroatoms selected from N, O and S. In the case of substituted heteraromatic rings containing at least one nitrogen atom (e.g., pyridine), such substitutions can be those resulting in N-oxide formation. Representative examples of heteroaromatic rings include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl.

Representative examples of bicyclic heterocycles include benzotriazolyl, indolyl, isoindolyl, indazolyl, indolinyl, isoindolinyl, quinoxalinyl, quinazolinyl, cinnolinyl, chromanyl, isochromanyl, tetrahydroquinolinyl, quinolinyl, tetrahydroisoquinolinyl, isoquinolinyl, 2,3-dihydrobenzofuranyl, 2,3-dihydrobenzo-1,4-dioxinyl, imidazo(2,1-b)(1,3)thiazole), and benzo-1,3-dioxolyl. In certain contexts herein, 2,3-dihydrobenzofuranyl, 2,3-dihydrobenzo-1,4-dioxinyl is alternatively referred to as phenyl having as a substituent methylenedioxy attached to 2 adjacent carbon atoms.

Unless otherwise specifically noted as only “unsubstituted” or only “substituted”, alkyl, cycloalkyl, aryl and heterocycle groups are unsubstituted. As used herein, the terms “substituted alkyl”, “substituted C₃-C₁₀ cycloalkyl”, “substituted aryl” and “substituted heterocycle” are intended to include the cyclic group containing from 1 to 3 substituents in addition to the point of attachment to the rest of the compound. Preferably, the substituents are selected from the group which includes, but is not limited to, halo, C₁-C₂₀ alkyl, —CF₃, —NH₂, —N(C₁-C₆ alkyl)₂, —NO₂, oxo, —CN, —N₃, —OH, —O(C₁-C₆ alkyl), C₃-C₁₀ cycloalkyl, C₂-C₆ alkenyl, C₂-C₆ alkynyl, (C₀-C₆ alkyl)-S(O)O₀₋₂—, aryl-S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆alkyl)O(C₁-C₆ alkyl)-, (C₀-C₆ alkyl)C(O)₁₋₂(C₀-C₆ alkyl)-, (C₀-C₆ alkyl)OC(O)NH—, aryl, aralkyl, heteroaryl, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl. Unless specifically indicated, such substituents themselves are not substituted.

Unless expressly stated to the contrary, substitution by a named substituent is permitted on any atom in a ring (e.g., aryl, a heteroaromatic ring, or a saturated heterocyclic ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain of the compounds of the present invention can have asymmetric centers and can occur as mixtures of stereoisomers, or as individual diastereomers, or enantiomers. All isomeric forms of these compounds, whether isolated or in mixtures, are within the scope of the present invention.

As would be recognized by one of ordinary skill in the art, certain of the compounds of the present invention can exist as tautomers. For the purposes of the present invention a reference to a compound of formula I is a reference to the compound per se, or to any one of its tautomers per se, or to mixtures of two or more tautomers.

When any variable (e.g., R⁴, R⁵ etc.) occurs more than one time in any constituent or in formula I or in any other formula depicting and describing compounds of the invention, its definition on each occurrence is independent of its definition at every other occurrence. Also, combinations of substituents and/or variables are permissible only if such combinations result in stable compounds.

Unless expressly stated to the contrary, all ranges cited herein are inclusive. For example, a heteroaryl ring described as containing from “1 to 3 heteroatoms” means the ring can contain 1, 2, or 3 heteroatoms. It is also to be understood that any range cited herein includes within its scope all of the sub-ranges within that range. The oxidized forms of the heteroatoms N and S are also included within the scope of the present invention.

The compounds of the present inventions are useful in the inhibition of HCV protease (e.g., HCV NS3 protease) and the treatment of HCV infection and/or reduction of the likelihood or severity of symptoms of HCV infection. For example, the compounds of this invention are useful in treating infection by HCV after suspected past exposure to HCV by such means as blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HCV protease, e.g., by competitive inhibition. Thus, the compounds of this invention are commercial products to be used for these purposes.

The compounds of the present invention may be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of the invention mean providing the compound or a prodrug of the compound to the individual in need of treatment. When a compound of the invention or a prodrug thereof is provided in combination with one or more other active agents (e.g., antiviral agents useful for treating HCV infection), “administration” and its variants are each understood to include concurrent and sequential provision of the compound or salt (or hydrate) and other agents.

As used herein, the term “prodrug” is intended to encompass a drug form or compound that is converted into an active drug form or compound by the action of enzymes, chemicals or metabolic processes in the body of an individual to whom it is administered.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition whose likelihood of occurrence or severity is being reduced. The term also includes herein the amount of active compound sufficient to inhibit HCV NS3 protease and thereby elicit the response being sought (i.e., an “inhibition effective amount”). When the active compound (i.e., active ingredient) is administered as the salt, references to the amount of active ingredient are to the free acid or free base form of the compound.

For the purpose of inhibiting HCV NS3 protease and preventing or treating HCV infection, the compounds of the present invention, optionally in the form of a salt or a hydrate, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in said compositions is provided in Remington's Pharmaceutical Sciences, 18^{th} edition, edited by A. R. Gennaro, Mack Publishing Co., 1990.

The compounds of this invention can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention also relates to a method of inhibiting HCV NS3 protease, inhibiting HCV replication, or preventing or treating HCV infection with a compound of the present invention in combination with one or more therapeutic agents and a pharmaceutical composition comprising a compound of the present invention and one or more therapeutic agents selected from the group consisting of a HCV antiviral agent, an immunomodulator, and an anti-infective agent. Such therapeutic agents active against HCV include, but are not limited to, ribavirin, levovirin, viramidine, thymosin alpha-1, RC025 (an enhanced interferon (Roche)), interferon-β, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffmann-LaRoche, Nutley, N.J.), pegylated interferon-α2a (PEGASYS), interferon-α2b (such as Intron-A interferon available from Schering Corp., Kenilworth, N.J.), pegylated interferon-α2b (PEGINTRON), a recombinant consensus interferon (such as interferon alphacon-1), albuferon (interferon-α bound to human serum albumin (Human Genome Sciences)), and a purified interferon-α product. Amgen's recombinant consensus interferon has the brand name INFERGEN. Levovirin is the L-enantiomer of ribavirin which has shown immunomodulatory activity similar to ribavirin. Viramidine represents an analog of ribavirin disclosed in WO 01/60379 (assigned to ICN Pharmaceuticals). In accordance with the method of the present invention, the individual components of the combination can be administered separately at different times during the course of therapy or concurrently in divided or single combination forms.

For the treatment of HCV infection, the compounds of the present invention may also be administered in combination with an agent that is an inhibitor of HCV NS3 serine protease. HCV NS3 serine protease is an essential viral enzyme and has been described to be an excellent target for inhibition of HCV replication. Both substrate and non-substrate based inhibitors of HCV NS3 protease inhibitors are disclosed in WO 98/22496, WO 98/46630, WO 99/07733, WO 99/07734, WO 99/38888, WO 99/50230, WO 99/64442, WO 00/09543, WO 00/59929, GB 2337262, WO 02/48116, WO 02/48172, and U.S. Pat. No. 6,323,180.

Ribavirin, levovirin, and viramidine may exert their anti-HCV effects by modulating intracellular pools of guanine nucleotides via inhibition of the intracellular enzyme inosine monophosphate dehydrogenase (IMPDH). IMPDH is the rate-limiting enzyme on the biosynthetic route in de novo guanine nucleotide biosynthesis. Ribavirin is readily phosphorylated intracellularly and the monophosphate derivative is an inhibitor of IMPDH. Thus, inhibition of IMPDH represents another useful target for the discovery of inhibitors of HCV replication. Therefore, the compounds of the present invention may also be administered in combination with an inhibitor of IMPDH, such as VX-497, which is disclosed in WO 97/41211 and WO 01/00622; another IMPDH inhibitor, such as that disclosed in WO 00/25780; or mycophenolate mofetil [see A. C. Allison and E. M. Eugui, Agents Action, 44 (Suppl.): 165 (1993)].

For the treatment of HCV infection, the compounds of the present invention may also be administered in combination with the antiviral agent amantadine (1-aminoadamantane) [for a comprehensive description of this agent, see J. Kirschbaum, Anal. Profiles Drug Subs. 12: 1-36 (1983)].

For the treatment of HCV infection, the compounds of the present invention may also be administered in combination with the antiviral agent polymerase inhibitor RC128 (Roche).

The compounds of the present invention may also be combined for the treatment of HCV infection with antiviral 2′-C-branched ribonucleosides disclosed in R. E. Harry-O'Kuru et al., J. Org. Chem., 62: 1754-1759 (1997); M. S. Wolfe et al., Tetrahedron Lett., 36: 7611-7614 (1995); U.S. Pat. No. 3,480,613; International Publication Numbers WO 01/90121, WO 01/92282, WO 02/32920, WO 04/002999, WO 04/003000, and WO 04/002422; the contents of each of which are incorporated by reference in their entirety. Such 2′-C-branched ribonucleosides include, but are not limited to, 2′-C-methyl-cytidine, 2′-C-methyl-uridine, 2′-C-methyl-adenosine, 2′-C-methyl-guanosine, and 9-(2-C-methyl-β-D-ribofuranosyl)-2,6-diaminopurine, and the corresponding amino acid ester of the ribose C-2′, C-3′, and C-5′ hydroxyls and the corresponding optionally substituted cyclic 1,3-propanediol esters of the 5′-phosphate derivatives.

The compounds of the present invention may also be combined for the treatment of HCV infection with other nucleosides having anti-HCV properties, such as those disclosed in WO 02/51425, WO 01/79246, WO 02/32920, WO 02/48165, and WO 2005003147 (including RL56, (2′R)-2′-deoxy-2′-fluoro-2′-C-methylcytidine, shown as compounds 3-6 on page 77); WO 01/68663, WO 99/43691, WO 02/18404, US 2005/0038240, WO 2006021341 (including 4′-azido nucleosides such as RL26, 4′-azidocytidine), US 2002/0019363, WO 02/100415, WO 03/026589, WO 03/026675, WO 03/093290, US 2003/0236216, US 2004/0006007, WO 04/011478, WO 04/013300, US 2004/0063658, and WO 04/028481; the content of each is incorporated herein by reference in its entirety.

For the treatment of HCV infection, the compounds of the present invention may also be administered in combination with an agent that is an inhibitor of HCV NS5B polymerase. Such HCV NS5B polymerase inhibitors that may be used as combination therapy include, but are not limited to, those disclosed in WO 02/057287, U.S. Pat. No. 6,777,395, WO 02/057425, US 2004/0067901, WO 03/068244, WO 2004/000858, WO 04/003138 and WO 2004/007512; the content of each is incorporated herein by reference in its entirety. Other such HCV polymerase inhibitors include, but are not limited to, valopicitabine (NM-283; Idenix) and 2′-F-2′-beta-methylcytidine (see also WO 2005/003147).

In one embodiment, nucleoside HCV NS5B polymerase inhibitors that are used in combination with the present HCV NS3 protease inhibitors are selected from the following compounds: 4-amino-7-(2-C-methyl-3β-D-arabinofuranosyl)-7H-pyrrolo[2,3-d]pyrimidine; 4-amino-7-(2-C-methyl-β-D-ribofuranosyl)-7H-pyrrolo[2,3-d]pyrimidine; 4-methylamino-7-(2-C-methyl-β-D-ribofuranosyl)-7H-pyrrolo[2,3-d]pyrimidine; 4-dimethylamino-7-(2-C-methyl-β-D-ribofuranosyl)-7H-pyrrolo[2,3-d]pyrimidine; 4-cyclopropylamino-7-(2-C-methyl-β-D-ribofuranosyl)-7H-pyrrolo[2,3-d]pyrimidine; 4-amino-7-(2-C-vinyl-β-D-ribofuranosyl)-7H-pyrrolo[2,3-d]pyrimidine; 4-amino-7-(2-C-hydroxymethyl-β-D-ribofuranosyl)-7H-pyrrolo[2,3-d]pyrimidine; 4-amino-7-(2-C-fluoromethyl-β-D-ribofuranosyl)-7H-pyrrolo[2,3-d]pyrimidine; 4-amino-5-methyl-7-(2-C-methyl-β-D-ribofuranosyl)-7H-pyrrolo[2,3-d]pyrimidine; 4-amino-7-(2-C-methyl-β-D-ribofuranosyl)-7H-pyrrolo[2,3-d]pyrimidine-5-carboxylic acid; 4-amino-5-bromo-7-(2-C-methyl-β-D-ribofuranosyl)-7H-pyrrolo[2,3-d]pyrimidine; 4-amino-5-chloro-7-(2-C-methyl-β-D-ribofuranosyl)-7H-pyrrolo[2,3-d]pyrimidine; 4-amino-5-fluoro-7-(2-C-methyl-β-D-ribofuranosyl)-7H-pyrrolo[2,3-d]pyrimidine; 2,4-diamino-7-(2-C-methyl-β-D-ribofuranosyl)-7H-pyrrolo[2,3-d]pyrimidine; 2-amino-7-(2-C-methyl-(3-D-ribofuranosyl)-7H-pyrrolo[2,3-d]pyrimidine; 2-amino-4-cyclopropylamino-7-(2-C-methyl-β-D-ribofuranosyl)-7H-pyrrolo[2,3-d]pyrimidine; 2-amino-7-(2-C-methyl-β-D-ribofuranosyl)-7H-pyrrolo[2,3-d]pyrimidin-4(3H)-one; 4-amino-7-(2-C-ethyl-β-D-ribofuranosyl)-7H-pyrrolo[2,3-d]pyrimidine; 4-amino-7-(2-C, 2-O-dimethyl-β-D-ribofuranosyl)-7H-pyrrolo[2,3-d]pyrimidine; 7-(2-C-methyl-β-D-ribofuranosyl)-7H-pyrrolo[2,3-d]pyrimidin-4(3H)-one; 2-amino-5-methyl-7-(2-C, 2-O-dimethyl-β-D-ribofuranosyl)-7H-pyrrolo[2,3-d]pyrimidin-4(3H)-one; 4-amino-7-(3-deoxy-2-C-methyl-β-D-ribofuranosyl)-7H-pyrrolo[2,3-d]pyrimidine; 4-amino-7-(3-deoxy-2-C-methyl-β-D-arabinofuranosyl)-7H-pyrrolo[2,3-d]pyrimidine; 4-amino-2-fluoro-7-(2-C-methyl-β-D-ribofuranosyl)-7H-pyrrolo[2,3-d]pyrimidine; 4-amino-7-(3-C-methyl-β-D-ribofuranosyl)-7H-pyrrolo[2,3-d]pyrimidine; 4-amino-7-(3-C-methyl-(3-D-xylofuranosyl)-7H-pyrrolo[2,3-d]pyrimidine; 4-amino-7-(2,4-di-C-methyl-β-D-ribofuranosyl)-7H-pyrrolo[2,3-d]pyrimidine; 4-amino-7-(3-deoxy-3-fluoro-2-C-methyl-β-D-ribofuranosyl)-7H-pyrrolo[2,3-d]pyrimidine; and the corresponding 5′-triphosphates; or a pharmaceutically acceptable salt thereof.

The compounds of the present invention may also be combined for the treatment of HCV infection with non-nucleoside inhibitors of HCV polymerase such as those disclosed in WO 01/77091, WO 01/47883, WO 02/04425, WO 02/06246, WO 02/20497, WO 2005/016927 (in particular, JTK003), and HCV-796 (ViroPharma Inc.); the content of each is incorporated herein by reference in its entirety.

In one embodiment, non-nucleoside HCV NS5B polymerase inhibitors that are used in combination with the present HCV NS3 protease inhibitors are selected from the following compounds: 14-cyclohexyl-6-[2-(dimethylamino)ethyl]-7-oxo-5,6,7,8-tetrahydroindolo[2,1-a][2,5]benzodiazocine-11-carboxylic acid; 14-cyclohexyl-6-(2-morpholin-4-ylethyl)-5,6,7,8-tetrahydroindolo[2,1-a][2,5]benzodiazocine-11-carboxylic acid; 14-cyclohexyl-6-[2-(dimethylamino)ethyl]-3-methoxy-5,6,7,8-tetrahydroindolo[2,1-a][2,5]benzodiazocine-11-carboxylic acid; 14-cyclohexyl-3-methoxy-6-methyl-5,6,7,8-tetrahydroindolo[2,1-a][2,5]benzodiazocine-11-carboxylic acid; methyl ({[(14-cyclohexyl-3-methoxy-6-methyl-5,6,7,8-tetrahydroindolo[2,1-a][2,5]benzodiazocin-11-yl)carbonyl]amino)}sulfonyl)acetate; ({[(14-cyclohexyl-3-methoxy-6-methyl-5,6,7,8-tetrahydroindolo[2,1-a][2,5]benzodiazocin-11-yl)carbonyl]amino}sulfonyl)acetic acid; 14-cyclohexyl-N-[(dimethylamino)sulfonyl]-3-methoxy-6-methyl-5,6,7,8-tetrahydroindolo[2,1-a][2,5]benzodiazocine-11-carboxamide; 3-chloro-14-cyclohexyl-6-[2-(dimethylamino)ethyl]-7-oxo-5,6,7,8-tetrahydroindolo[2,1-a][2,5]benzodiazocine 11-carboxylic acid; N′-(11-carboxy-14-cyclohexyl-7,8-dihydro-6H-indolo[1,2-e][1,5]benzoxazocin-7-yl)-N,N-dimethylethane-1,2-diaminium bis(trifluoroacetate); 14-cyclohexyl-7,8-dihydro-6H-indolo[1,2-e][1,5]benzoxazocine-11-carboxylic acid; 14-cyclohexyl-6-methyl-7-oxo-5,6,7,8-tetrahydroindolo[2,1-a][2,5]benzodiazocine-11-carboxylic acid; 14-cyclohexyl-3-methoxy-6-methyl-7-oxo-5,6,7,8-tetrahydroindolo[2,1-a][2,5]benzodiazocine-11-carboxylic acid; 14-cyclohexyl-6-[2-(dimethylamino)ethyl]-3-methoxy-7-oxo-5,6,7,8-tetrahydroindolo[2,1-a][2,5]benzodiazocine-11-carboxylic acid; 14-cyclohexyl-6-[3-(dimethylamino)propyl]-7-oxo-5,6,7,8-tetrahydroindolo[2,1-a][2,5]benzodiazocine-11-carboxylic acid; 14-cyclohexyl-7-oxo-6-(2-piperidin-1-ylethyl)-5,6,7,8-tetrahydroindolo[2,1-a][2,5]benzodiazocine-11-carboxylic acid; 14-cyclohexyl-6-(2-morpholin-4-ylethyl)-7-oxo-5,6,7,8-tetrahydroindolo[2,1-a][2,5]benzodiazocine-11-carboxylic acid; 14-cyclohexyl-6-[2-(diethylamino)ethyl]-7-oxo-5,6,7,8-tetrahydroindolo[2,1-a][2,5]benzodiazocine-11-carboxylic acid; 14-cyclohexyl-6-(1-methylpiperidin-4-yl)-7-oxo-5,6,7,8-tetrahydroindolo[2,1-a][2,5]benzodiazocine-11-carboxylic acid; 14-cyclohexyl-N-[(dimethylamino)sulfonyl]-7-oxo-6-(2-piperidin-1-ylethyl)-5,6,7,8-tetrahydroindolo[2,1-a][2,5]benzodiazocine-11-carboxamide; 14-cyclohexyl-6-[2-(dimethylamino)ethyl]-N-[(dimethylamino)sulfonyl]-7-oxo-5,6,7,8-tetrahydroindolo[2,1-a][2,5]benzodiazocine-11-carboxamide; 14-cyclopentyl-6-[2-(dimethylamino)ethyl]-7-oxo-5,6,7,8-tetrahydroindolo[2,1-a][2,5]benzodiazocine-11-carboxylic acid; 14-cyclohexyl-5,6,7,8-tetrahydroindolo[2,1-a][2,5]benzodiazocine-11-carboxylic acid; 6-allyl-14-cyclohexyl-3-methoxy-5,6,7,8-tetrahydroindolo[2,1-a][2,5]benzodiazocine-11-carboxylic acid; 14-cyclopentyl-6-[2-(dimethylamino)ethyl]-5,6,7,8-tetrahydroindolo[2,1-a][2,5]benzodiazocine-11-carboxylic acid; 14-cyclohexyl-6-[2-(dimethylamino)ethyl]-5,6,7,8-tetrahydroindolo[2,1-a][2,5]benzodiazocine-11-carboxylic acid; 13-cyclohexyl-5-methyl-4,5,6,7-tetrahydrofuro[3′,2′:6,7][1,4]diazocino[1,8-a]indole-10-carboxylic acid; 15-cyclohexyl-6-[2-(dimethylamino)ethyl]-7-oxo-6,7,8,9-tetrahydro-5H-indolo[2,1-a][2,6]benzodiazonine-12-carboxylic acid; 15-cyclohexyl-8-oxo-6,7,8,9-tetrahydro-5H-indolo[2,1-a][2,5]benzodiazonine-12-carboxylic acid; 13-cyclohexyl-6-oxo-6,7-dihydro-5H-indolo[1,2-d][1,4]benzodiazepine-10-carboxylic acid; and pharmaceutically acceptable salts thereof.

The HCV NS3 protease inhibitory activity of the present compounds may be tested using assays known in the art. One such assay is HCV NS3 protease time-resolved fluorescence (TRF) assay as described below and in International Patent Application Publication WO2006/102087. Other examples of such assays are described in e.g., International Patent Application Publication WO2005/046712. HCV NS3 protease inhibitors, such as those described herein have a Ki less than 50 μM, such as less than 10 μM, and less than 100 nM. Ki is determined by an NS3 protease assay. The assay is performed in a final volume of 100 μl in assay buffer containing 50 mM HEPES, pH 7.5, 150 mM NaCl, 15% glycerol, 0.15% TRITON X-100, 10 mM DTT, and 0.1% PEG 8000. NS3 protease is pre-incubated with various concentrations of inhibitors in DMSO for 30 minutes. The reaction is initiated by adding the TRF peptide substrate (final concentration 100 nM). NS3 mediated hydrolysis of the substrate is quenched after 1 hour at room temperature with 100 μl of 500 mM MES, pH 5.5. Product fluorescence is detected using either a VICTOR V2 or FUSION fluorophotometer (Perkin Elmer Life and Analytical Sciences) with excitation at 340 nm and emission at 615 nm with a 400 μs delay. Testing concentrations of different enzyme forms are selected to result in a signal to background ratio (S/B) of 10-30. IC₅₀ values are derived using a standard four-parameter fit to the data. Kᵢ values are derived from IC₅₀ values using the following formula,

IC₅₀=Kᵢ(1+[S]/K_{M}), Eqn (1),

where [S] is the concentration of substrate peptide in the reaction and K_{M} is the Michaelis constant. See P. Gallinari et al., 38 BIOCHEM. 5620-32 (1999); P. Gallinari et al., 72 J. VIROL. 6758-69 (1998); M. Taliani et al., 240 ANAL. BIOCHEM. 60-67 (1996).

The present invention also includes processes for making compounds of formula (I). The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. The following reaction schemes and examples serve only to illustrate the invention and its practice.

Olefin metathesis catalysts include the following Ruthenium-based species: Scott J. Miller et al., Application of Ring-Closing Metathesis to the Synthesis of Rigidified Amino Acids and Peptides, 118 J. AM. CHEM. SOC. 9606 (1996); Jason S. Kingsbury et al., A Recyclable Ru-Based Metathesis Catalyst, 121 J. AM. CHEM. SOC. 791 (1999); Matthias Scholl et al., Synthesis and Activity of a New Generation of Ruthenium-Based Olefin Metathesis Catalysts Coordinated with 1,3-Dimesityl-4,5-dihydroimidazol-2-ylidene Ligands, 1(6) ORGANIC LETTERS 953 (1999); US 2002/0107138; Alois Fiirstner et al., Total Synthesis and Structural Refinement of the Cyclic Tripyrrole Pigment Nonylprodigiosin, 64 J. ORG. CHEM. 8275 (1999). The utility of these catalysts in ring-closing metathesis is well known in the literature (e.g. Tina M. Trnka & Robert H. Grubbs, The Development of L₂X₂Ru═CHR Olefin Metathesis Catalysts: An Organometallic Success Story, 34 ACC. CHEM. RES. 18 (2001)).

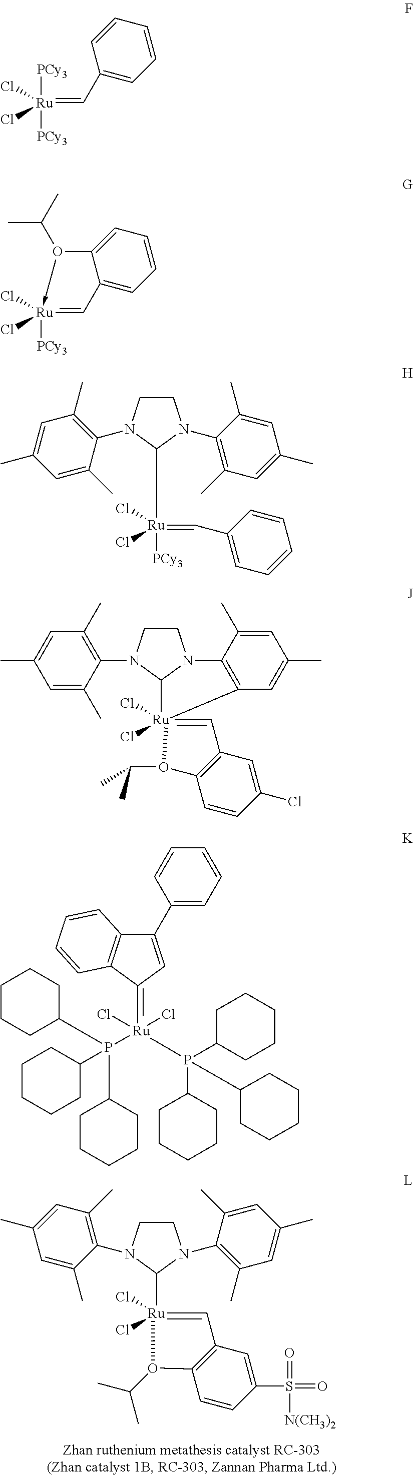

The following examples serve only to illustrate the invention and its practice. The examples are not to be construed as limitations on the scope or spirit of the invention.

### EXAMPLES

### Abbreviations

BOC (also Boc) t-Butyloxycarbonyl
B(OMe)₃ Trimethyl borate
BOP Benzotriazole-1-yl-oxy-tris-(dimethylamino)-phosphonium hexafluorophosphate
Brosyl chloride 4-Bromophenyl sulfonylchloride
tBuOH t-Butanol
BuLi Butyl lithium
CAN Ceric ammonium nitrate
CDCl₃ Deuterio-trichloromethane
CDI N,N′-Carbonyl diimidazole
CH₃CN Acetonitrile
mCPBA m-Chloroperbenzoic acid
Cs₂CO₃ Cesium carbonate
CuI Copper iodide
Cu(I)Br.SMe₂ Copper (I) bromide dimethyl sulfide complex
DABCO 1,4-diazabicyclo[2.2.2]octane
DBA (also dba) Dibenzylidene acetone
DBU 1,8-Diazabicyclo[5.4.0]undec-7-ene
DCC Dicyclohexylcarbodiimide
DCE Dichloroethane
DCM Dichloromethane
DEAD Diethyl azodicarboxylate
DIAD Diisopropyl azodicarboxylate
DIEA Diethylamine
DIPA Diethylpropylamine
DIPEA Diisopropylethylamine
DMAP 4-Dimethylamino pyridine
DMF Dimethylformamide
DMSO Dimethyl Sulfoxide
DPPF (also dppf) 1,1′-bid(Diphenylphosphino)ferrocene
EDC N-(3-Dimethylaminopropyl)-N′-ethylcarbodiimide
ESI Electrospray ionization
Et₂O Diethyl ether
EtOAc Ethyl Acetate
EtOH Ethanol
H₂ Hydrogen or hydrogen atmosphere
HATU O-(7-Azabenzotriazol-1-yl)-N,N,N′,N′-tetramethyluronium hexafluorophosphate
HBr Hydrobromic acid
HCl Hydrochloric acid
HMPA Hexamethylphosphoramide
HOAc Acetic acid
HOAt 1-Hydroxy-7-azabenzotriazole
HOBT 1-Hydroxy benzotriazole
H₂O Water
H₂O₂Hydrogen peroxide
HPLC High performance liquid chromatography
I₂ Iodine
KHSO₄ Potassium bisulfate
K₂SO₄ Potassium sulfate
K₂CO₃ Potassium carbonate
KOH Potassium hydroxide
LAH Lithium aluminium hydride
LCMS High performance liquid chromatography-mass spectrometry
LiOH Lithium hydroxide
LiOH.H₂O Lithium hydroxide monohydrate
LRMS Low resolution mass spectrometry
Me₃Al Trimethylaluminium
MeLi Methyllithium
MeOH Methanol
MgSO₄ Magnesium Sulfate
MsCl Mesyl chloride
N₂ Nitrogen or nitrogen atmosphere
NH₄Cl Ammonium chloride
NH₄OH Ammonium hydroxide
Nle Norleucine
NMP N-Methyl pyrrolidinone
NaH Sodium hydride
NaHCO₃ Sodium hydrogen carbonate (sodium bicarbonate)
NaHSO₃ Sodium bisulfite
NaOH Sodium hydroxide
NaOMe Sodium methoxide
Na₂SO₃ Sodium sulfite
Na₂S₂O₃ Sodium thiosulfate
Na₂SO₄ Sodium sulfate (anhydrous)
PCy₃ Tricyclohexyl phosphine
POBr Phosphoryl bromide
POBr₃ Phosphoryl tribromide
P₂O₅ phosphorus pentoxide (P₄O₁₀)
Pd/C Palladium on carbon
PhMe Toluene
PPh₃ Triphenylphosphine
RT Room temperature, approximately 25 C
Ru/C Ruthenium on carbon
SiO₂ Silica or silica gel
TBAF Tetrabutylammonium fluoride
TBTU O-Benzotriazol-1-yl-N,N,N′,N′-tetramethyluronium tetrafluoroborate
TEA Triethylamine
TFA Trifluoroacetic acid
THF Tetrahydrofuran
TIPSOTf Triisopropylsilyl triflate
TMSCl Chlorotrimethyl silane
TsCl p-Toluenesulfonyl chloride
Zn(CN)₂ Zinc cyanide

### Synthesis of Intermediates

### Intermediates A

| Intermediate | | | Literature |
|---|---|---|---|
| Number | Structure | Name | Reference |
| A1 | 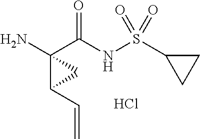 | (1R,2S)-1-Amino-N- (cyclopropylsulfonyl)-2- vinylcyclopropanecarboxamide hydrochloride | U.S. Pat. No. 6,995,174 |
| A2 | 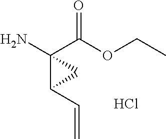 | Ethyl (1R,2S)-1-amino-2- vinylcyclopropanecarboxylate hydrochloride | U.S. Pat. No. 6,323,180 |

### Intermediate A3: (1R,2R)-1-Amino-N-(cyclopropylsulfonyl)-2-ethylcelopropanecarboxamide hydrochloride

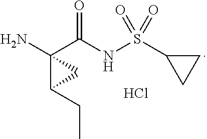

### Step 1: t-Butyl((1R,2R)-1-{[(cyclopropylsulfonyl)amino]carbonyl}-2-ethylcyclopropyl)carbamate

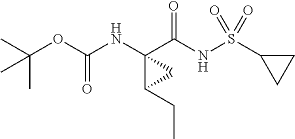 [SMILES: CC[C@@H]1C[C@]1(NC(=O)OC(C)(C)C)C(=O)NS(=O)(=O)C1CC1]

A hydrogenation vessel was charged with a MeOH (1000 mL) slurry of t-butyl ((1R,2S)-1-{[(cyclopropylsulfonyl)amino]carbonyl}-2-vinylcyclopropyl)carbamate (164 g, 0.50 mol) (U.S. Pat. No. 6,995,174) and 5% Ru/C (dry, 7.5 wt %, 12.4 g) and stirred. The vessel was placed under N₂ (20 psi) and vented to atmospheric pressure (3×) to remove residual oxygen. The vessel was then placed under H₂ (50 psi). After 20 hours, the vessel was vented to atmospheric pressure. The reaction slurry was then transferred out of the reaction vessel and filtered through SOLKA FLOK (34 g, wetted with 100 mL MeOH) to yield a clear, light brown solution. The SOLKA FLOK was rinsed with MeOH (200 mL×2). The combined MeOH solutions were concentrated under reduced pressure to yield crude product as a white solid (153 g). The crude product was slurried in EtOAc (800 mL), warmed to 40° C. and aged minutes. The solution was then seeded, aged 30 minutes, and heptane (500 mL) was added via addition funnel over 30 minutes. The partially crystallized solid was cooled to RT and aged overnight, after which additional heptane (500 mL) was added. After 1 hour, additional heptane (250 mL) was added via addition funnel, and the white slurry aged for 1 hour. The solution was filtered, and the solid was rinsed with heptane/EtOAc (500 mL, 4:1) and dried under reduced pressure to give t-butyl ((1R,2R)-1-{[(cyclopropylsulfonyl)amino]carbonyl}-2-ethylcyclopropyl)carbamate (125.9 g).

### Step 2: (1R,2R)-1-Amino-N-(cyclopropylsulfonyl)-2-ethylcyclopropanecarboxamide hydrochloride (Intermediate A3)

A solution of the product from Step 1 (92 g, 0.28 mol) in DCM (1200 mL) was cooled to 0° C., and HCl was bubbled through the solution for 10 minutes. The cooling bath was then removed, and the reaction mixture stirred for 2 hours. N₂ was bubbled through the reaction mixture for 5 minutes, and the volatiles evaporated. The residue was azeotroped with DCM (3×) to give an off-white powder (75 g). LRMS (M+H)⁺ calculated=233; found 233.

### Intermediate A4: (1R,2S)-1-amino-2-ethenyl-N-[(1-methylcyclopropyl)sulfonyl]cyclopropane-carboxamide hydrochloride

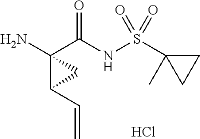 [SMILES: C=C[C@@H]1C[C@]1(N)C(=O)NS(=O)(=O)C1(C)CC1 ; Cl]

### Step 1: N-tert-butylcyclopropanesulfonamide

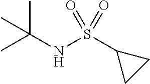

To solution of t-butylamine (17.3 mL, 164 mmol) in THF (100 mL) cooled to −20° C., cyclopropylsulfonyl chloride (11.55 g, 82 mmol) was added. The mixture was then warmed to RT and stirred for 20 hours. The resulting solid was removed by filtration, and the solvent was removed in vacuo. The residue was then dissolved in DCM and washed with 1 N HCl, dried over MgSO₄, and the solvent was removed in vacuo. The resulting material was crystallized from hexanes to yield the title compound as a white solid (14.5 g).

### Step 2: N-tert-butyl-1-methylcyclopropanesulfonamide

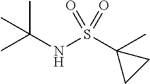

To a solution of the product from step 1 (8.6 g, 48.8 mmol) in THF (160 mL) cooled to −78° C., n-BuLi (42.9 mL, 2.5 M, 107 mmol) was added, while keeping the internal temperature less than −40° C. The mixture was then warmed to 6° C. over 20 minutes, re-cooled to −78° C., and iodomethane (6.1 mL, 98 mmol) was added. After stirring for a further 2 hours, the reaction was quenched with aqueous NH₄Cl, extracted with EtOAc, dried over sodium sulfate, and the solvent was removed in vacuo. The resulting material was titrated with hexanes to yield the title compound as an off-white powder (7.0 g).

### Step 3: 1-methylcyclopropanesulfonamide (ref: WO 20080502; Commercially available from Asia Tech #64790)

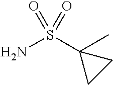

To the product from step 2 (7.0 g, 36.9 mmol) was added TFA (40 mL) and the resulting solution was stirred for 18 hours. The solvent was then removed in vacuo, and the resulting solid was crystallized from 1:4 EtOAc/hexanes to yield the title compound as a white solid (4.4 g).

### Step 4: tert-butyl [(1R,2S)-2-ethenyl-1-{[(1-methylcyclopropyl)sulfonyl]carbamoyl}cyclopropyl]carbamate

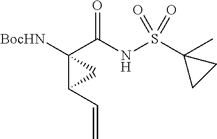 [SMILES: C=C[C@@H]1C[C@]1(NC(=O)OC(C)(C)C)C(=O)NS(=O)(=O)C1(C)CC1]

To a solution of (1R,2S)-1-[(tert-butoxycarbonyl)amino]-2-ethenylcyclopropanecarboxylic acid (4.35 g, 19.1 mmol) in THF (75 mL), carbonyldiimidazole (4.0 g, 24.88 mmol) was added, and the solution was then heated to reflux for 1 hour. The mixture was then cooled to RT, and the product from step 3 (4.35 g, 19.14 mmol) was then added followed by DBU (4.0 mL, 26.8 mmol). After stirring for 20 hours, 1 N HCl was added until acidic, and the mixture was extracted with EtOAc 2×. The combined organic layers were dried over Na₂SO₄, and the solvent was removed in vacuo. 1:1 EtOAc/hexanes (100 mL) was then added, and the resulting solid was filtered. The filtrate was concentrated and the 1:1 EtOAc/hexanes was added again to produce more solid. The combined solids were dried to yield the title compound as a solid (5 g). LCMS (ES+) m/z 289.3 ((M-t-Bu)+H)⁺.

### Step 5: (1R,2S)-1-amino-2-ethenyl-N-[(1-methylcyclopropyl)sulfonyl]cyclopropanecarboxamide hydrochloride

To a solution of the product from step 4 (7.8 g, 22.7 mmol) in DCM (30 mL), TFA (30 mL) was added. After 1.5 hours, 4 N HCl in EtOAc was added, and the volitiles were removed in vacuo. The resulting material was dissolved in DCM/MeOH, and the solvent was removed in vacuo. This material was then titrated with DCM to give the title compound as an off-white solid (5.7 g). LCMS (ES+) m/z 245.3 (M+H)⁺.

### Intermediate A5: 1-tert-Butyl 2-methyl (2S,4S)-4-{[(4-bromophenyl)sulfonyl]oxy}pyrrolidine-1,2-dicarboxylate

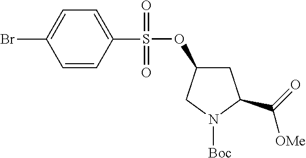 [SMILES: COC(=O)[C@@H]1C[C@H](OS(=O)(=O)c2ccc(Br)cc2)CN1C(=O)OC(C)(C)C]

A solution of brosyl chloride (3.14 g, 12.3 mmol) in PhMe (5 mL) was added to a solution of 1-tert-butyl 2-methyl (2S,4S)-4-hydroxypyrrolidine-1,2-dicarboxylate (2.15 g, 8.76 mmol) and DABCO (1.57 g, 14.0 mmol) in PhMe (10 mL) at RT. A white precipitate formed; the reaction mixture was stirred for 20 minutes and filtered. The filtrate was partitioned between EtOAc and saturated aqueous NaHCO₃. The layers were separated, and the organic layer was washed with 1 M HCl, water and brine, dried over Na₂SO₄, filtered and concentrated. The title compound (4.0 g, 98% yield) was then used without further purification. LRMS (M+Na)⁺ calculated: 488; found 488.

### Intermediates B

### Intermediate B1: (1R,2R)-2-allyl-1-methylcyclopropanol and (1S,2S)-2-allyl-1-methylcyclopropanol

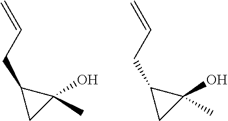

To a mixture of titanium(IV) isoproproxide (50.9 ml, 174 mmol) in THF (170 ml) at RT was added methylmagnesium bromide (260 ml, 260 mmol) over 5 minutes. The resulting solution was allowed to stir 5 minutes. The solution was cooled to −5° C. and dry EtOAc (17.00 ml, 174 mmol) in THF (30 mL) was added. The reaction was stirred for 1 minute, and a solution of pentenylmagnesium bromide (0.76M in THF, 347 ml, 347 mmol) was added dropwise over 60 minutes from an addition funnel while maintaining the temperature at 0-5° C. The reaction was allowed to warm to RT and then aged for 1.5 hour. The reaction was cooled to −5° C. and quenched by the dropwise addition of 10% aq. H₂SO₄ (600 mL) while the temperature was maintained at 0-10° C. The reaction was extracted with Et₂O (2×), then the organics were combined, washed with 10% aq. NaHCO₃, brine, dried over MgSO₄, filtered and concentrated to give a crude oil. The oil was purified on SiO₂ (gradient elution, 0-20% EtOAc/hexane) to afford the title compound as an oil (12 g, 61.6%).

### Intermediate B2: (1R,2R)-2-(but-3-en-1-yl)-1-methylcyclopropanol and (1S,2S)-2-(but-3-en-1-yl)-1-methylcyclopropanol

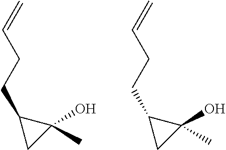

To a solution of EtOAc (12.9 mL, 132 mL) in THF (658 mL), 1,5-hexadiene (32.4 g, 395 mmol) and chlorotitanium triisopropoxide (132 mL, 1M, 132 mmol) were added. Cyclohexylmagnesium chloride (296 mL, 2M, 592 mmol) was then added over 2 hours via an addition funnel. The reaction was then stirred for an additional 1 hour. The mixture was then cooled in an ice bath and quenched with H₂O (27 mL), 15% NaOH (27 mL), and more H₂O (60 mL). Na₂SO₄ was then added; the mixture was filtered through CELITE, and the solvent was removed in vacuo. The crude product was purified by silica (gradient elution 10:1 to 4:1 hexanes/EtOAc) to yield the title compounds (11.5 g).

### Intermediate B3: (1R,2R)-1-methyl-2-(pent-4-en-1-yl)cyclopropanol and (1S,2S)-1-methyl-2-(pent-4-en-1-yl)cyclopropanol

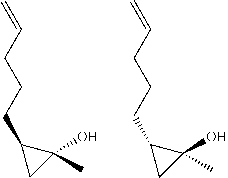

The title compounds were prepared according to the procedure for Intermediate B2 using 1,6-heptadiene in place of 1,5-hexadiene.

### Intermediate B3-1: (1R,2R)-1-methyl-2-(pent-4-en-1-yl)cyclopropanol (single enantiomer of Intermediate B3)

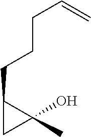

To a solution of 1,6-heptadiene (19.65 g, 204 mmol), in THF (341 ml) was added EtOAc (6.67 ml, 68.1 mmol), and chlorotitanium triisopropoxide (68.1 ml, 68.1 mmol). Cyclohexylmagnesium chloride (2M, 153 ml, 306 mmol) was then added slowly over 2 hours. After an additional 1 hour of stirring at RT, the reaction mixture was filtered through CELITE. The filtrate was then concentrated in vacuo and purified on SiO₂ (15% EtOAc/hexanes) to yield the title compound as a mixture of enantiomers. To a solution of the enantiomers (5.5 g, 39.2 mmol) in anhydrous DCM, at 0° C. and under N₂, was added Et₃N (10.9 mL, 78 mmol) and 4-nitrobenzoyl chloride (8.7 g, 47.1 mmol). The reaction was stirred for 2 hours, diluted with H₂O (70 mL) and extracted with DCM (3×100 mL). The combined organic layer was dried over Na₂SO₄, filtered and concentrated in vacuo. The residue was purified on SiO₂ (gradient elution, 0 to 50% EtOAc in hexane) to yield (1R,2R and 1S,2S)-1-methyl-2-(pent-4-en-1-yl)cyclopropyl 4-nitrobenzoate. The benzoate enantiomers (12 g, 41.5 mmol) were separated by chiral chromatography on an AD-H column, eluting with 10% CO₂ in MeOH. The desired peak fractions were concentrated to give (1R,2R)-1-methyl-2-(pent-4-en-1-yl)cyclopropyl 4-nitrobenzoate. To a solution of (1R,2R)-1-methyl-2-(pent-4-en-1-yl)cyclopropyl 4-nitrobenzoate (4.87 g, 16.83 mmol) in MeOH (67 mL) was added K₂CO₃ (4.65 g, 33.7 mmol). The mixture was stirred for 1 hour, filtered, and concentrated in vacuo. The material was purified on SiO₂ (gradient elution, 0-50% ether/hexanes) to give the title compound. ¹H NMR (500 MHz) (CDCl₃) δ 5.82 (m, 1H), 4.99 (m, 2H), 2.09 (m, 2H), 1.78 (s, 1H), 1.55 (m, 2H), 1.41 (s, 3H), 1.32 (m, 1H), 1.14 (m, 1H), 0.97 (m, 1H), 0.84 (m, 1H), 0.06 (m, 1H).

### Intermediate B4: (1R,2S)-2-(but-3-en-1-yl)-1-methylcyclopentanol and (1S,2R)-2-(but-3-en-1-yl)-1-methylcyclopentanol

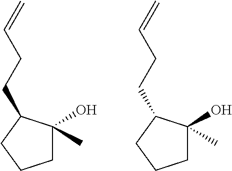

To a 0.5 M solution of 3-butenylmagnesium bromide (45.6 mL, 22.8 mmol), copper(I) chloride (113 mg, 1.14 mmol) was added at 0° C. A solution of methylcyclopentene oxide (1.12 g, 11.4 mmol) in diethyl ether (5 mL) was added to the resulting purple solution. The mixture was then warmed to RT overnight, and then cooled to 0° C. and poured into a saturated solution of NH₄Cl also cooled to 0° C. The reaction mixture was then extracted with EtOAc, dried over MgSO₄, and the solvent was removed in vacuo. The crude material was purified on silica (gradient elution, 0-30% EtOAc/hexanes) to yield the title compounds (1.3 g).

### Intermediate B5: (1R,2R)-1-methyl-2-(pent-4-en-1-yl)cyclopentanol and (1 SR²S)-1-methyl-2-(pent-4-en-1-yl)cyclopentanol

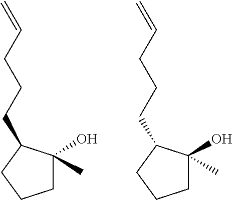

The title compounds were prepared according to the procedure for Intermediate B4 using 4-pentenylmagnesium bromide.

### Intermediates C

### Intermediate C1: (2S)-[({[(1R,2R)-2-allyl-1-methylcyclopropyl]oxy}carbonyl)amino](cyclopentyl)acetic acid and (2S)-[({[(1S,2S)-2-allyl-1-methylcyclopropyl]oxy}carbonyl)amino](cyclopentyl)acetic acid

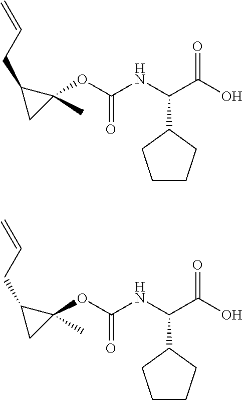 [SMILES: C=CC[C@@H]1C[C@@]1(C)OC(=O)N[C@H](C(=O)O)C1CCCC1 ; C=CC[C@H]1C[C@]1(C)OC(=O)N[C@H](C(=O)O)C1CCCC1]

### Step 1: methyl (2S)-cyclopentyl(isocyanato)acetate

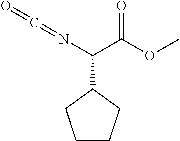

To a mixture of methyl (2S)-amino(cyclopentyl)acetate hydrochloride (50 g, 258 mmol) in DCM (1229 ml), sodium bicarbonate (108 g, 1291 mmol) dissolved in water (1 L) was added, and the mixture was stirred well with an overhead stirrer for 5 minutes. The reaction was cooled to 0° C., and triphosgene (25.3 g, 85 mmol) was added. The reaction was stirred vigorously at 0° C. for 1.5 hours. The reaction was poured into a separatory furnnel, extracted aqueous with DCM, combined organics, washed with brine, dried over MgSO₄, filtered, concentrated in vacuo to yield the title compound as a colorless oil (43 g, 91%).

### Step 2: Methyl (2S)-[({[(1R,2R)-2-allyl-1-methylcyclopropyl]oxy}carbonyl)amino](cyclopentyl)acetate and Methyl (2S)-[({[(1S,2S)-2-allyl-1-methylcyclopropyl]oxy}carbonyl)amino](cyclopentyl)acetate

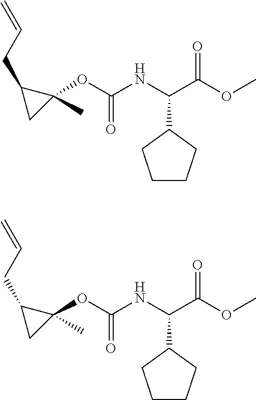 [SMILES: C=CC[C@@H]1C[C@@]1(C)OC(=O)N[C@H](C(=O)OC)C1CCCC1 ; C=CC[C@H]1C[C@]1(C)OC(=O)N[C@H](C(=O)OC)C1CCCC1]

A mixture of intermediate B1 (12.0 g, 107 mmol), methyl (2S)-cyclopentyl(isocyanato)acetate (27.4 g, 150 mmol), DIPEA (74.7 ml, 428 mmol) and DMAP (13.07 g, 107 mmol) in toluene (350 ml) was heated to 85° C. and stirred for 24 hours. The reaction was diluted with Et₂O (500 mL) and 1 M HCl (1500 mL), stirred well, and the organic layer was removed. The aqueous portion was extracted with ether, and the organics were combined, washed with brine, dried over Na₂SO₄, filtered through a pad of silica and washed with ether. The filtrate was concentrated to give crude oil, which was purified on SiO₂ (gradient elution, 0-30% EtOAc/hexane) to afford the title compound as an oil (25 g, 79%).

### Step 3: (2S)-[({[(1R,2R)-2-allyl-1-methylcyclopropyl]oxy}carbonyl)amino](cyclopentyl)acetic acid and (2S)-[({[(1S,2S)-2-allyl-11-methylcyclopropyl]oxy}carbonyl)amino](cyclopentyl)acetic acid

Intermediate from step 2 (42.5 g, 144 mmol) was dissolved in THF (750 ml) and treated with lithium hydroxide (17.23 g, 719 mmol) dissolved in H₂O (250 ml). The mixture was allowed to stir at RT for 3 hours. The reaction mixture was treated with 3N HCL (240 mL), concentrated to remove THF, and diluted with aq. KHSO₄ (200 mL), and the product was extracted into DCM (3×300 mL). The DCM layer was dried over magnesium sulfate, filtered, and concentrated to yield the title compound as a white solid (40.5 g, 100%).

### Intermediate C2: (2S)-[({[(1R,2R)-2-(but-3-en-1-yl)-1-methylcyclopropyl]oxy}carbonyl)amino](cyclopentyl)ethanoic acid and (2S)-[({[(1S,2S)-2-(but-3-en-1-yl)-1-methylcyclopropyl]oxy}carbonyl)amino](cyclopentyl)ethanoic acid

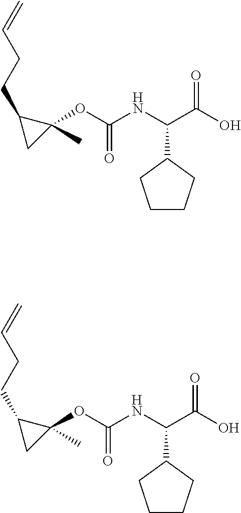 [SMILES: C=CCC[C@@H]1C[C@@]1(C)OC(=O)N[C@H](C(=O)O)C1CCCC1 ; C=CCC[C@H]1C[C@]1(C)OC(=O)N[C@H](C(=O)O)C1CCCC1]

Using Intermediate B2, the title compounds were prepared according to the procedure used for Intermediate C1.

### Intermediate C3: (2S)-cyclopentyl[({[(1R,2R)-1-methyl-2-(pent-4-en-1-yl)cyclopropyl]oxy}carbonyl)amino]ethanoic acid and (2S)-cyclopentyl[({[(1S,2S)-1-methyl-2-(pent-4-en-1-yl)cyclopropyl]oxy}carbonyl)amino]ethanoic acid

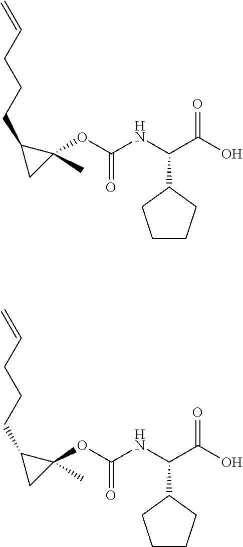 [SMILES: C=CCCC[C@@H]1C[C@@]1(C)OC(=O)N[C@H](C(=O)O)C1CCCC1 ; C=CCCC[C@H]1C[C@]1(C)OC(=O)N[C@H](C(=O)O)C1CCCC1]

Using Intermediate B3, the title compounds were prepared according to the procedure used for Intermediate C1.

### Intermediate C3-1: (2S)-cyclopentyl[({[1R,2R)-1-methyl-2-(pent-4-en-1-yl)cyclopropyl]oxy}carbonyl)amino]ethanoic acid

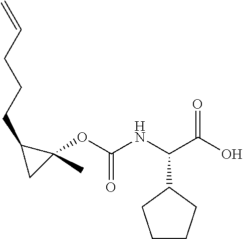 [SMILES: C=CCCC[C@@H]1C[C@@]1(C)OC(=O)N[C@H](C(=O)O)C1CCCC1]

The title compound was prepared according to the procedure for Intermediate C1 using Intermediate B3-1.

### Intermediate C4: 3-methyl-N-({[(1R,2R)—1-methyl-2-(pent-4-en-1-yl)cyclopropyl]oxy}carbonyl)-L-valine and 3-methyl-N-({[(1S,2S)-1-methyl-2-(pent-4-en-1-yl)cyclopropyl]oxy}carbonyl)-L-valine

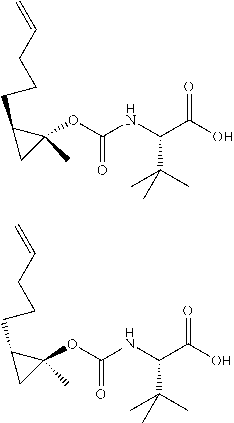 [SMILES: C=CCCC[C@@H]1C[C@@]1(C)OC(=O)N[C@H](C(=O)O)C(C)(C)C ; C=CCCC[C@H]1C[C@]1(C)OC(=O)N[C@H](C(=O)O)C(C)(C)C]

Using Intermediate B3 and methyl 3-methyl-L-valinate hydrochloride, the title compounds were prepared according to the procedure used for Intermediate C1.

### Intermediate C4-1: 3-methyl-N-({[(1R,2R)-1-methyl-2-(pent-4-en-1-yl)cyclopropyl]oxy}carbonyl)-L-valine

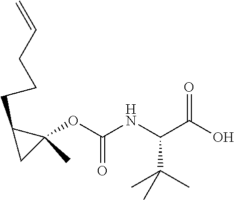 [SMILES: C=CCCC[C@@H]1C[C@@]1(C)OC(=O)N[C@H](C(=O)O)C(C)(C)C]

The title compound was prepared according to the procedure for Intermediate C4 using B3-1.

### Intermediate C5: (2S)-[({[(1R,2S)-2-(but-3-en-1-yl)-1-methylcyclopentyl]oxy}carbonyl)amino](cyclopentyl)ethanoic acid and (2S)-[({[(1S,2R)-2-(but-3-en-1-yl)-1-methylcyclopentyl]oxy}carbonyl)amino](cyclopentyl)ethanoic acid

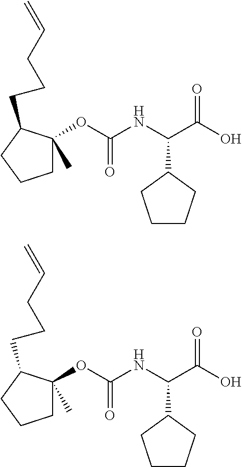 [SMILES: C=CCCC[C@@H]1CCC[C@@]1(C)OC(=O)N[C@H](C(=O)O)C1CCCC1 ; C=CCCC[C@H]1CCC[C@]1(C)OC(=O)N[C@H](C(=O)O)C1CCCC1]

Using Intermediate B4, the title compounds were prepared according to the procedure used for Intermediate C1.

### Intermediate C6: (2S)-cyclopentyl[({[(1R,2R)-1-methyl-2-(pent-4-en-1-yl)cyclopentyl]oxy}carbonyl)amino]ethanoic acid and (2S)-cyclopentyl[({[(1R,2R)-1-methyl-2-(pent-4-en-1-yl)cyclopentyl]oxy}carbonyl)amino]ethanoic acid

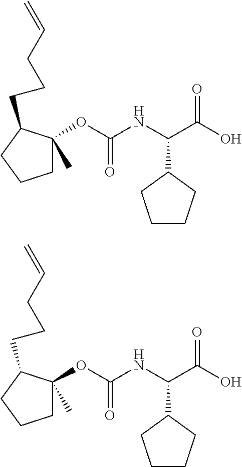 [SMILES: C=CCCC[C@@H]1CCC[C@@]1(C)OC(=O)N[C@H](C(=O)O)C1CCCC1 ; C=CCCC[C@H]1CCC[C@]1(C)OC(=O)N[C@H](C(=O)O)C1CCCC1]

The title compounds were prepared according to procedure used for Intermediate C1 using Intermediate B5.

### Intermediate C7: N-({[(1R,2R)-2-(but-3-en-1-yl)-1-methylcyclopropyl]oxy}carbonyl)-3-methyl-L-valine and N-({([(1S,2S)-2-(but-3-en-1-yl)-1-methylcyclopropyl]oxy}carbonyl)-3-methyl-L-valine

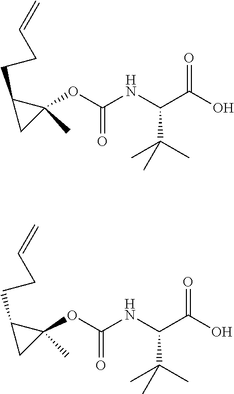 [SMILES: C=CCC[C@@H]1C[C@@]1(C)OC(=O)N[C@H](C(=O)O)C(C)(C)C ; C=CCC[C@H]1C[C@]1(C)OC(=O)N[C@H](C(=O)O)C(C)(C)C]

Using Intermediate B2 and methyl 3-methyl-L-valinate hydrochloride, the title compounds were prepared according to the procedure used for Intermediate C1.

### Intermediates D

### Intermediate D1: (2S,4R)-4-[(2-bromo-6-methoxyquinolin-3-yl)oxy]-2-(methoxycarbonyl)pyrrolidinium chloride

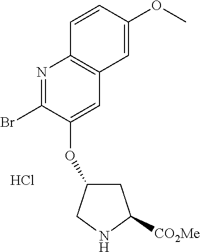 [SMILES: COC(=O)[C@@H]1C[C@@H](Oc2cc3cc(OC)ccc3nc2Br)CN1 ; Cl]

### Step 1: 2-Bromo-6-methoxyquinoline

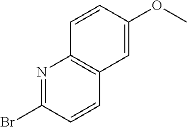

6-Methoxyquinolin-2(1H)-one (6.81 g, 38.9 mmol) was carefully added to POBr₃ (18.9 g, 66.1 mmol) at 60° C., and the resulting solution was stirred at 140° C. for 2.5 hours. The reaction mixture was cooled and poured onto crushed ice, and the solid was collected by filtration. Purification of this material on SiO₂ (gradient elution, 5-12% EtOAc/petroleum ether) afforded the title compound (4.57 g, 49.3%) as a solid. LCMS (ES+) m/z 238, 240 (M+H)⁺.

### Step 2: (2-Bromo-6-methoxyquinolin-3-yl)boronic acid

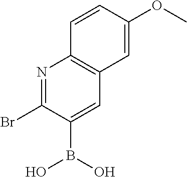 [SMILES: COc1ccc2nc(Br)c(B(O)O)cc2c1]

n-BuLi (1.6 N in hexanes, 14.4 mL, 23.0 mmol) was added at −78° C. to a solution of 2,2,6,6-tetramethylpiperidine (3.11 g, 22.05 mmol) in anhydrous THF (59 mL), and the mixture was then warmed to 0° C. for 30 minutes. The mixture was cooled back to −78° C. and treated with a solution of 2-bromo-6-methoxyquinoline (4.57 g, 19.17 mmol) in THF (14 mL).

After stirring for 1 hour, a solution of B(OMe)₃ (2.46 mL, 22.05 mmol) in THF (14 mL) was added, and the mixture was maintained at −78° C. for a further 2 hours. A mixture of THF (14 mL) and H₂O (3.5 mL) was added, then the solution was warmed to −10° C. and treated with H₂O (70 mL) and Et₂O (70 mL). NaOH_{(aq.)} (1N, 75 mL) was added, and the aqueous layer was separated and acidified to pH 4 with HCl_{(aq.)} (3N). The aqueous phase was extracted with Et₂O, and the combined extracts were washed with brine and dried over Na₂SO₄. Filtration and removal of the volatiles afforded the title compound (4.64 g, 86% yield) as an oily solid that was used directly in the subsequent step. LCMS (ES+) m/z 282, 284 (M+H)⁺.

### Step 3: 2-Bromo-6-methoxyquinolin-3-ol

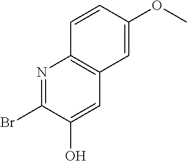

H₂O₂(aq.) (30%, 32.8 mL, 321 mmol) was added dropwise to a stirred solution of (2-bromo-6-methoxyquinolin-3-yl)boronic acid (4.64 g, 16.45 mmol) and NH₄Cl (3.29 g, 61.5 mmol) in Et₂O (82 mL) and H₂O (82 mL). After 13 hours, NH₄Cl (3.29 g, 61.5 mmol) and H₂O_{2(aq.)} (30%, 32.8 mL, 321 mmol) were added, and the mixture was stirred for 48 hours. The precipitate was collected and washed with H₂O, then dried at 50° C. to afford the title compound (4.18 g, 100%) as a solid that was used directly in the subsequent step. LCMS (ES+) m/z 254, 256 (M+H)⁺.

### Step 4: 1-t-Butyl 2-methyl (2S,4R)-4-[(2-bromo-6-methoxyquinolin-3-yl)oxy]pyrrolidine-1,2-dicarboxylate

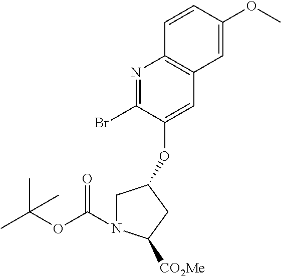 [SMILES: COC(=O)[C@@H]1C[C@@H](Oc2cc3cc(OC)ccc3nc2Br)CN1C(=O)OC(C)(C)C]

Cs₂CO₃ (10.7 g, 32.9 mmol) was added to a stirred mixture of 1-t-butyl 2-methyl (2S,4S)-4-{[(4-bromophenyl)sulfonyl]oxy}pyrrolidine-1,2-dicarboxylate (Intermediate A5) (8.78 g, 18.9 mmol) and 2-bromo-6-methoxyquinolin-3-ol (4.18 g, 16.45 mmol) in NMP (46 mL). The resulting mixture was stirred at 50° C. for 3 hours, then cooled and diluted with EtOAc, The organics were washed with saturated NaHCO_{3(aq.)}, H₂O and brine, then dried over Na₂SO₄. Filtration and removal of the volatiles gave a residue that was purified by column chromatography on SiO₂ (gradient elution, 1-100% EtOAc/petroleum ether) to give the title compound (5.56 g, 70.2%). LCMS (ES+) m/z 481, 483 (M+H)⁺.

### Step 5: (2S,4R)-4-[(2-bromo-6-methoxyquinolin-3-yl)oxy]-2-(methoxycarbonyl)pyrrolidinium chloride

A solution of 1-t-butyl 2-methyl (2S,4R)-4-[(2-bromo-6-methoxyquinolin-3-yl)oxy]pyrrolidine-1,2-dicarboxylate (5.01 g, 10.40 mmol) in HCl/dioxane (4 N, 31 ml) was prepared at 0° C., and the mixture was stirred at 20° C. for 40 minutes. The volatiles were evaporated, and the residue was triturated with Et₂O to afford an approximately 1:1 mixture of the title compound and (2S,4R)-4-[(2-chloro-6-methoxyquinolin-3-yl)oxy]-2-(methoxycarbonyl)pyrrolidinium chloride (4.34 g) as a solid that was used directly in subsequent steps. LCMS (ES+) m/z 381, 383 (M+H)⁺.

### Intermediate D2: (2S,4R)-4-[(3-chloroquinoxalin-2-yl)oxy]-2-(methoxycarbonyl)pyrrolidinium chloride

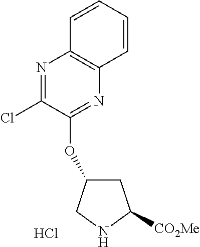 [SMILES: COC(=O)[C@@H]1C[C@@H](Oc2nc3ccccc3nc2Cl)CN1 ; Cl]

### Step 1: 1-t-Butyl 2-methyl (2S,4R)-4-[(3-chloroquinoxalin-2-yl)oxy]pyrrolidine-1,2-dicarboxylate

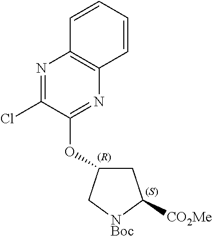

A solution of 3-chloroquinoxalin-2-ol (1.44 g, 7.97 mmol) and 1-t-butyl 2-methyl (2S,4S)-4-hydroxypyrrolidine-1,2-dicarboxylate (2.05 g, 8.37 mmol) in THF (190 ml) was cooled to 0° C., then treated with PPh₃ (2.51 g, 9.57 mmol). DIAD (1.86 ml, 9.57 mmol) was added dropwise, and the mixture was stirred at 20° C. for 1 hour. After evaporation of the volatiles, the residue was purified on SiO₂ (gradient elution, 0-70% EtOAc/petroleum ether) to afford the title compound (2.5 g, 77%). LCMS (ES+) m/z 408 (M+H)⁺.

### Step 2: (2S,4R)-4-[(3-chloroquinoxalin-2-yl)oxy]-2-(methoxy)-2-(methoxycarbonyl)pyrrolidinium chloride

A solution of 1-t-butyl 2-methyl (2S,4R)-4-[(3-chloroquinoxalin-2-yl)oxy]pyrrolidine-1,2-dicarboxylate (1.05 g, 2.57 mmol) in HCl/dioxane (4 N, 5 mL) was prepared at 0° C., then stirred for 2 hours at 20° C. The reaction mixture was concentrated to afford a residue that was triturated with Et₂O to afford the title compound (0.88 g, 98%) as a white solid that was used directly in subsequent reactions. LCMS (ES+) m/z 308 (M+H)⁺.

### Intermediate D3: Methyl (4R)-4-[(3-but-3-en-1-yl-8-methoxyquinoxalin-2-yl)oxy]-L-prolinate dihydrochloride

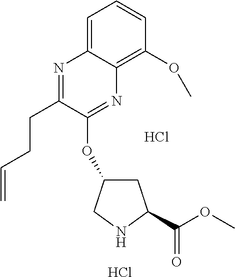 [SMILES: C=CCCc1nc2cccc(OC)c2nc1O[C@H]1CN[C@H](C(=O)OC)C1 ; Cl ; Cl]

### Step 1: 2-methoxy-6-nitroaniline

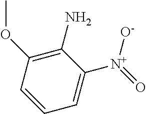

A mixture of 2-amino-3-nitrophenol (11.66 g, 76 mmol) and K₂CO₃ (12.55 g, 91 mmol) were stirred in DMF (100 ml) for 1 hour. Iodomethane (5.68 ml, 91 mmol) in DMF (10 mL) was added dropwise, and the mixture was stirred 14 hours. The reaction was diluted with H₂O and extracted with EtOAc (2×). The organics were washed with brine, dried over Na₂SO₄, filtered, concentrated to a dark solid. The crude solid was purified by crystallization from hexane to give the title compound as an orange solid (11.6 g, 91%). LCMS (ES+) m/z 169.0 (M+H)⁺.

### Step 2: 3-methoxybenzene-1,2-diamine

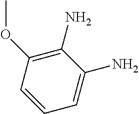

To a degassed solution of 2-methoxy-6-nitroaniline from step 1 (6.9 g, 41.0 mmol) in EtOH (200 ml) Pd/C (0.873 g, 0.821 mmol) was added, and the mixture was placed under a hydrogen atmosphere (balloon). After 6.5 hours, the reaction was filtered through a CELITE pad, and the filtrate was concentrated to yield the crude title compound as an orange oil (5.6 g, 99%).

### Step 3: 3-but-3-en-1-yl-8-methoxyquinoxalin-2-ol

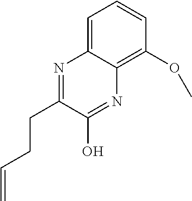 [SMILES: C=CCCc1nc2cccc(OC)c2nc1O]

To a solution of 3-methoxybenzene-1,2-diamine from step 2 (5.6 g, 40.5 mmol) in DCE (250 ml), ethyl 2-oxohex-5-enoate (9.50 g, 60.8 mmol) was added, and the mixture was stirred at 60° C. for 14 hours. An additional charge of ethyl 2-oxohex-5-enoate (1.45 g, 9.28 mmol) was added, and the mixture was heated for 24 hours. The reaction mixture was concentrated and filtered from CH₂Cl₂ to remove a portion of the undesired regio-isomer as a tan solid. The filtrate was purified by column chromatography on SiO₂ (gradient elution, 1-9% acetone/CH₂Cl₂) to give the title compound as a tan solid (6.4 g, 68.6%) after triturating with 10% Et₂O/hexane. LCMS (ES+) m/z 231.1 (M+H)+.

### Step 4: 1-tert-butyl 2-methyl (2S,4R)-4-[(3-but-3-en-1-yl-8-methoxyquinoxalin-2-yl)oxy]pyrrolidine-1,2-dicarboxylate

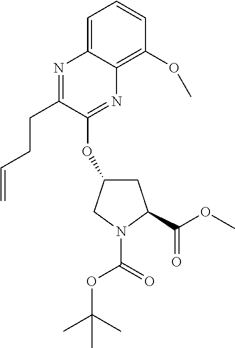 [SMILES: C=CCCc1nc2cccc(OC)c2nc1O[C@@H]1C[C@@H](C(=O)OC)N(C(=O)OC(C)(C)C)C1]

To a solution of 3-but-3-en-1-yl-8-methoxyquinoxalin-2-ol from step 3 (6.15 g, 26.7 mmol) in NMP (200 ml), 1-t-butyl 2-methyl (2S,4S)-4-{[(4-bromophenyl)sulfonyl]oxy}pyrrolidine-1,2-dicarboxylate (12.40 g, 26.7 mmol) and cesium carbonate (26.1 g, 80 mmol) were added. The resulting slurry was warmed to 50° C. for 5 hours while under N₂. An additional amount of 1-t-butyl 2-methyl (2S,4S)-4-{[(4-bromophenyl)sulfonyl]oxy}pyrrolidine-1,2-dicarboxylate (3.7 g, 7.96 mmol) and cesium carbonate (2.5 g, 7.7 mmol) were added, and heating was continued for 14 hours. The reaction was diluted with EtOAc (200 mL) and filtered, the filtrate was quenched into 10% KHSO₄, and the product was extracted into EtOAc (2×). The organics were combined, washed with brine (2×), dried over sodium sulfate, filtered, and concentrated to a brown oil. Purification by column chromatography on SiO₂ (gradient elution, 20-45% EtOAc/hexane) gave the title compound as a colorless oil (9.4 g, 77%). LCMS (ES+) m/z 458.0 (M+H)⁺.

### Step 5: Methyl (4R)-4-[(3-but-3-en-1-yl-8-methoxyquinoxalin-2-yl)oxy]-L-prolinate dihydrochloride

A solution of 1-tert-butyl 2-methyl (2S,4R)-4-[(3-but-3-en-1-yl-8-methoxyquinoxalin-2-yl)oxy]pyrrolidine-1,2-dicarboxylate from step 4 (9.4 g, 20.55 mmol) in HClidioxane (4 N, 150 mL) was prepared at 0° C., then stirred for 2 hours at 20° C. The reaction mixture was concentrated to afford a residue that was triturated with Et₂O to afford the title compound (9.5 g, 100%) as a white solid that was used directly in subsequent reactions. LCMS (ES+) m/z 358.0 (M+H)⁺.

### Intermediate D4: (3R,5S)-5-(Methoxycarbonyl)pyrrolidin-3-yl 4-vinyl-1,3-dihydro-2H-isoindole-2-carboxylate Hydrochloride

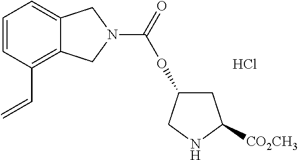 [SMILES: C=Cc1cccc2c1CN(C(=O)O[C@H]1CN[C@H](C(=O)OC)C1)C2 ; Cl]

### Step 1: 1-Bromo-2,3-bis(bromomethyl)benzene

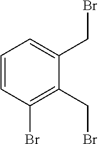 [SMILES: BrCc1cccc(Br)c1CBr]

A suspension of 3-bromo-o-xylene (196 g, 1.06 mol), N-bromosuccinimide (377 g, 2.15 mol) and benzoyl peroxide (0.26 g, 1.0 mmol) in carbon tetrachloride (1800 mL) was heated to reflux under N₂ for 15 hours. The contents of the reaction flask were cooled and filtered, and the filtrate was evaporated. The crude material was distilled under high vacuum; the major fractions were distilled between 88° C. and 152° C. From these distillates, 108 g of pure material was recovered, and 182 g of slightly crude material, which could be used in the following reaction, was also recovered. ¹H NMR (CDCl₃) δ 7.56 (d, J=8.0 Hz, 1H), 7.31 (d, J=8.0 Hz, 1H), 7.26 (s, 1H), 7.16 (t, J=8.0 Hz, 1H), 4.84 (s, 2H), 4.64 (s, 2H) ppm.

### Step 2: 2-Benzyl-4-bromoisoindoline

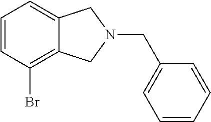 [SMILES: Brc1cccc2c1CN(Cc1ccccc1)C2]

KHSO₄ (204 g, 2.04 mol) was suspended in CH₃CN (12 L), and the mixture was heated to 80° C. Solutions of 1-bromo-2,3-bis(bromomethyl)benzene (280 g, 0.82 mol in 500 mL CH₃CN) and benzylamine (87.5 g, 0.82 mol in 500 mL CH₃CN) were added concurrently via addition funnels over 1 hour. The reaction mixture was stirred at 77° C. for 16 hours. The contents of the reaction flask were cooled and filtered, and the solvent was removed by evaporation. The reaction was partitioned between 1M K₂CO₃ and EtOAc. The organics were washed with brine, dried with anhydrous Na₂SO₄, filtered and evaporated. Flash column chromatography (gradient elution: heptane to 10% EtOAc in heptane) gave, after evaporation, the title compound as a pale oil. ¹H NMR (CDCl₃) δ 7.41-7.39 (m, 2H), 7.37-7.34 (m, 2H), 7.32-7.27 (m, 2H), 7.10-7.03 (m, 2H), 4.02 (s, 2H), 3.97 (s, 2H), 3.91 (s, 2H). LRMS (ESI) m/z 289 [(M+H)⁺; calculated for C₁₅H₁₅BrN: 289].

The product was converted to HCl salt in HCl/MeOH by the addition of MTBE and filtration of the solid to give 118 g of product as the HCl salt.

### Step 3: 2-Benzyl-4-vinylisoindoline

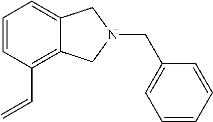 [SMILES: C=Cc1cccc2c1CN(Cc1ccccc1)C2]

A solution of 2-benzyl-4-bromoisoindoline (16.7 g, 58.0 mmol) and tributyl(vinyl)tin (20.3 mL, 69.6 mmol) in PhMe (400 mL) was degassed by bubbling N₂ gas through the solution for 0.25 hour. Tetrakis(triphenylphosphine)palladium (0) (1.30 g, 1.16 mmol) was added, and the resulting solution heated in a 100° C. oil bath under N₂ for 24 h.

The contents of the reaction flask were cooled, evaporated and subjected to flash column chromatography eluting with hexane/EtOAc 95/5 to give after evaporation the title compound as a pale oil that turned pink on standing. LRMS (ESI) m/z 236 [(M+H)⁺; calculated for C₁₇H₁₈N: 236].

### Step 4: 4-Vinylisoindoline

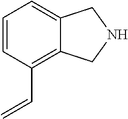

A solution of 2-benzyl-4-vinylisoindoline (58 mmol) in 1,2-dichloroethane (150 mL) was placed in a 1 L round-bottom flask under N₂. An addition funnel containing a solution of 1-chloroethyl chloroformate (7.51 mL, 69.6 mmol) in 1,2-dichloroethane was attached to the reaction flask. The reaction flask was cooled in an ice bath, and the contents of the addition funnel were added dropwise over 20 minutes, keeping the internal reaction temperature<5° C. After the addition was complete, the reaction flask was allowed to warm to RT, then heated to reflux for 45 minutes. The contents of the reaction flask were cooled to RT, then the solvent was removed by evaporation. MeOH (200 mL) was added, and the contents of the reaction flask were heated to reflux for 30 minutes. The reaction flask was cooled, and the solvent removed by evaporation. H₂O (200 mL) was added, and the resulting mixture washed with EtOAc (2×250 mL). The aqueous layer was made basic with 2N NaOH then extracted with methylene chloride (4×250 mL). The combined organic extracts were dried with anhydrous Na₂SO₄, filtered and the filtrate evaporated. The remaining residue was subjected to flash column chromatography eluting with methylene chloride/MeOH/NH₄OH 97/3/0.3 to 95/5/0.5. Evaporation of fractions gave the title compound as a brown oil, 6.00 g (41.4 mmol, 71% yield for two steps). LRMS (ESI) m/z 146 [(M+H)⁺; calculated for C₁₀H₁₂N: 146].

### Step 5: 1-tert-Butyl 2-methyl (2S,4R)-4-{[(4-vinyl-1,3-dihydro-2H-isoindol-2-yl)carbonyl]oxy}pyrrolidine-1,2-dicarboxylate

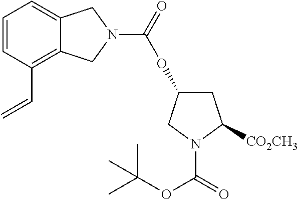 [SMILES: C=Cc1cccc2c1CN(C(=O)O[C@@H]1C[C@@H](C(=O)OC)N(C(=O)OC(C)(C)C)C1)C2]

A solution of 1-tert-butyl 2-methyl (2S,4R)-4-hydroxypyrrolidine-1,2-dicarboxylate (10.1 g, 41.4 mmol) in DMF (90 mL) under N₂ was cooled to 0° C. Solid 1,1′-carbonyldiimidazole (6.70 g, 41.4 mmol) was added to the reaction. The contents of the reaction flask were warmed to RT, and, after 2 hours, a solution of 4-vinylisoindoline (6.00 g, 41.4 mmol) in DMF (10 mL) was added. The reaction was heated in a 60° C. oil bath for 2 hours, then cooled and poured into H₂O and 5% KHSO₄. The resulting mixture was extracted with EtOAc (4×250 mL). The combined organics were washed with brine, dried with anhydrous Na₂SO₄, filtered and evaporated. Flash column chromatography eluting with hexane/EtOAc 70/30 gave the title compound as a white foam, 13.9 g (33.4 mmol, 81% yield). LRMS (ESI) m/z 417 [(M+H)⁺; calculated for C₂₂₇H₂₉N₂O₆: 417].

### Step 6: (3R,5S)-5-(Methoxycarbonyl)pyrrolidin-3-yl 4-vinyl-1,3-dihydro-2H-isoindole-2-carboxylate hydrochloride

A solution of 1-tert-Butyl 2-methyl (2S,4R)-4-{[(4-vinyl-1,3-dihydro-2H-isoindol-2-yl)carbonyl]oxy}pyrrolidine-1,2-dicarboxylate (13.9 g, 33.4 mmol) in EtOAc (700 mL) was cooled in an ice bath the saturated with HCl gas. The reaction flask was sealed and allowed to warm to RT. After 3.5 hours, the solvent was removed by evaporation to give the title compound as a gray solid, 11.2 g, 95% yield). ¹H NMR (500 MHz, CD₃OD) δ 7.47-7.45 (m, 1H), 7.32-7.31 (m, 1H), 7.26-7.21 (m, 1H), 6.79-6.73 (m, 1H), 5.79-5.73 (m, 1H), 5.46 (s, 1H), 5.41-5.38 (min, 1H), 4.80-4.72 (m, 4H), 3.91 (s, 3H), 3.74-3.63 (m, 2H), 2.77-2.71 (m, 1H), 2.51-2.46 (m, 1H). LRMS (ESI) m/z 317 [(M+H)⁺; calculated for C₁₇H₂₁N₂O₄: 317].

### Intermediate D5: methyl (4R)-4-{[7-chloro-4-oxo-3-(prop-2-en-1-yl-3,4-dihydroquinazolin-2-yl]oxy}-L-prolinate

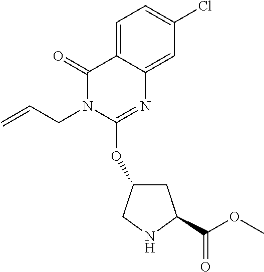 [SMILES: C=CCn1c(O[C@H]2CN[C@H](C(=O)OC)C2)nc2cc(Cl)ccc2c1=O]

### Step 1: 4-chloro-2-[(ethoxycarbonyl)amino]benzoic acid

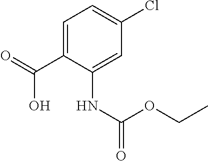 [SMILES: CCOC(=O)Nc1cc(Cl)ccc1C(=O)O]

To a solution of 2-amino-4-chlorobenzoic acid (30 g, 175 mmol) in THF (480 mL) at RT, ethyl chloroformate (50.4 mL, 525 mmol) was added. The mixture was heated to reflux overnight. The reaction was then concentrated in vacuo to an thick oil/foam that was flushed with PhMe, and the residue was slurried with ˜500 mL 5% ether/hexane. The resulting solids were filtered and washed with 5% ether/hexanes to give upon air drying the title compound (39 g). LRMS (ESI) m/z 244 [(M+H)⁺; calculated for C₁₀H₁₁ClNO₄: 244].

### Step 2: 7-chloro-3-(prop-2-en-1-yl)quinazoline-2,4(1H,3H)-dione

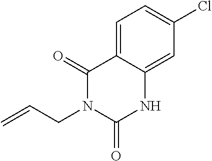 [SMILES: C=CCn1c(=O)[nH]c2cc(Cl)ccc2c1=O]

To a solution of the product from step 1 (39.0 g, 160 mmol), N-methylmorpholine (35.2 mL, 320 mmol) and allylamine (14.97 mL, 200 mmol) in DMF (50 mL) at RT, BOP reagent (88 g, 200 mmol) was added. The mixture was stirred at RT over the weekend. DBU (121 mL, 800 mmol) was then added, and the mixture was heated to 60° C. for 3 hours. The mixture was then poured into 0.5 N HCl (3000 mL), and the pH was adjusted to 2.0 with 3N HCl. Thick white solids observed, and the reaction was diluted to ˜4000 mL and let stir 10 minutes. The solids were filtered, washed with 0.5 N HCl, and then slurry washed with water and sucked dry. The title compound was obtained after air drying overnight (36.3 g). LRMS (ESI) m/z 237 [(M+H)⁺; calculated for C₁₁H₁₀ClN₂O₂: 237].

### Step 3: 2,7-dichloro-3-(prop-2-en-1-yl)quinazolin-4(3H)-one

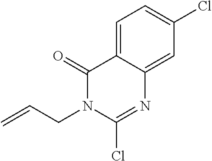 [SMILES: C=CCn1c(Cl)nc2cc(Cl)ccc2c1=O]

To the product from step 2 (10.25 g, 43.3 mmol), phosphorous oxychloride (161 mL, 1732 mmol) and N,N-dimethylaniline (10.98 mL, 87 mmol) were added. The mixture was heated to'reflux for 36 hours, concentrated in vacuo, and poured into ice water. The mixture was then extracted four times with EtOAc, and the combined organic portions were washed with water then brine, dried with anhydrous magnesium sulfate, filtered and concentrated in vacuo. The crude material was purified on silica (10% EtOAc/hexanes) to give a yellow solid that was triturated with 10% EtOAc/hexanes to give the title compound as a tan solid (7.5 g). LRMS (ESI) m/z 256 [(M+H)⁺; calculated for C₁₁H₉Cl₂N₂O: 256].

### Step 4: (4R)-1-(tert-butoxycarbonyl)-4-{[7-chloro-4-oxo-3-(prop-2-en-1-yl)-3,4-dihydroquinazolin-2-yl]oxy}-L-proline

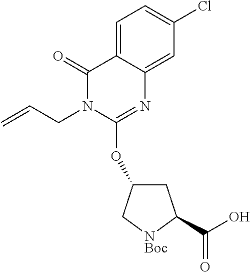 [SMILES: C=CCn1c(O[C@@H]2C[C@@H](C(=O)O)N(C(=O)OC(C)(C)C)C2)nc2cc(Cl)ccc2c1=O]

To a solution of Boc-L-4-Hydroxyproline (5.44 g, 23.52 mmol) in DMF (100 mL) cooled reaction in an cold bath to −60° C. was added NaHMDS (39.2 mL, 39.2 mmol) slowly. The mixture thickens and solids precipitate. The reaction was then warmed to 0° C. for 15 minutes and then re-cooled to −60° C. The product from step 3 (4 g, 15.68 mmol), as a solid, was then added in one portion. The reaction was then warmed slowly to 0° C. After 30 minutes, the mixture was poured into 10% KHSO₄ (125 mL) cooled to 0° C. The mixture was then extracted with EtOAc and the combined organic portions were washed with water 3× then brine, dried with anhydrous magnesium sulfate, filtered and concentrated in vacuo to give the title compound (7 g), which was used crude in the next step. LRMS (ESI) m/z 393 [((M-t-Bu)+H)⁺; calculated for C₁₇H₁₇ClN₃O₆: 393].

### Step 5: 1-tert-butyl 2-methyl (2S,4R)-4-{[7-chloro-4-oxo-3-(prop-2-en-1-yl)-3,4-dihydroquinazolin-2-yl]oxy}pyrrolidine-1,2-dicarboxylate

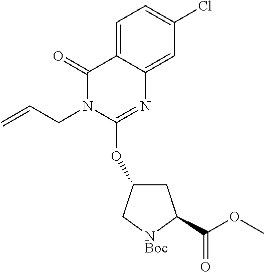 [SMILES: C=CCn1c(O[C@@H]2C[C@@H](C(=O)OC)N(C(=O)OC(C)(C)C)C2)nc2cc(Cl)ccc2c1=O]

To a solution of the crude product from step 6 (14.1 g, 31.3 mmol) in DCM (157 mL) and MeOH (157 mL) cooled in an ice bath, TMS-diazomethane (47 mL, 94 mmol) was added slowly, keeping the internal temperature<10° C. The mixture was then quenched by dropwise addition of 5% potassium bisulfate. The reaction was then concentrated in vacuo, water was added, and the mixture was extracted with EtOAc 3×. The combined organics were washed with brine, dried over MgSO₄, and the solvent was removed in vacuo. The crude material was purified on silica (gradient elution, 0-50% EtOAc/hexanes) to yield the title compound (13.9 g). LRMS (ESI) m/z 408 [((M-t-Bu)+H)⁺; calculated for C₁₈H₁₉ClN₃O₆: 408].

### Step 6: methyl (4R)-4-{[7-chloro-4-oxo-3-(prop-2-en-1-yl)-3,4-dihydroquinazolin-2-yl]oxy}-L-prolinate

To a solution of the product from step 5 (5.2 g, 11.25 mmol) in DCM (37.5 mL), TFA (37.5) was added. After 30 minutes, the solvents were removed in vacuo, and the crude material was taken up in DCM, washed with NaHCO₃ and dried over MgSO₄, and the solvent was removed in vacuo to give the title compound (4.27 g). LRMS (ESI) m/z 364 [(M+H)⁺; calculated for C₁₇H₁₉ClN₃O₄: 364].

### Intermediate D6: methyl (4R)-4-{[7-methyl-4-oxo-3-(prop-2-en-1-yl)-3,4-dihydroquinazolin-2-yl]oxy}-L-prolinate

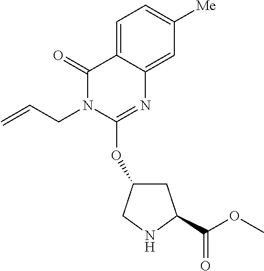 [SMILES: C=CCn1c(O[C@H]2CN[C@H](C(=O)OC)C2)nc2cc(C)ccc2c1=O]

The title compound was prepared according to the procedure used for Intermediate D5 using 2-amino-4-methylbenzoic acid in step 1.

### Intermediate D7: methyl (4R)-4-{[4-benzyloxy)-3-(prop-2-en-1-yl)quinolin-2-yl]oxy}-L-prolinate

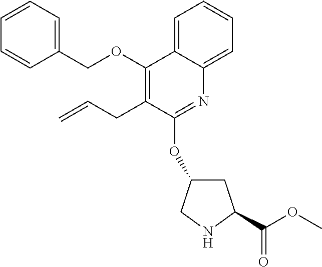 [SMILES: C=CCc1c(O[C@H]2CN[C@H](C(=O)OC)C2)nc2ccccc2c1OCc1ccccc1]

### Step 1: 3-(prop-2-en-1-yl)quinoline-2,4-diol

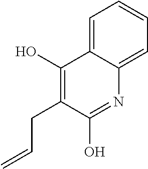

To a degassed solution of 2,4-dihydroxyquinoline (10.0 g, 62.1 mmol) in DMF (100 mL), TEA (9.51 mL, 68.3 mmol) and Pd(Ph₃P)₄ (2.151 g, 1.862 mmol) were added, followed by allyl acetate (7.43 mL, 68.3 mmol), and the mixture was allowed to stir at 60° C. under N₂ overnight. The reaction was cooled to RT and quenched into water (600 mL) and ˜80 mL sat Na₂CO₃. The pH was adjusted to 12, and DCM was added (500 mL). The aqueous layer was washed with another portion of DCM (500 mL). The aqueous layer was cooled in an ice bath, and pH adjusted to ˜2.5 with 12N HCl slowly to give pink solids, which were filtered and washed with water to give the title compound (11.5 g). LRMS (ESI) m/z 202 [(M+H)⁺; calculated for C₁₂H₁₂NO₂: 202].

### Step 2: 4-(benzyloxy)-3-(prop-2-en-1-yl)quinolin-2-ol

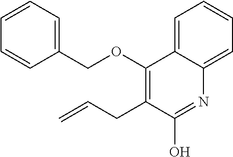 [SMILES: C=CCc1c(O)nc2ccccc2c1OCc1ccccc1]

To a mixture of the product from step 1 (4.0 g, 19.88 mmol), triphenylphosphine (6.78 g, 25.8 mmol), and benzyl alcohol (2.274 mL, 21.87 mmol) in THF (240 mL) cooled to 0° C., a THF (12 mL) solution of diisopropylazodicarboxylate (5.02 mL, 25.8 mmol) was slowly added dropwise. Upon completion of addition, the mixture was allowed to stir at RT for 60 minutes, and then it was concentrated in vacuo to give a thick oil. The residue was dissolved in DCM (˜20 mL), and solids then precipitated out of solution. The solids were filtered and washed with DCM (˜10 mL) to yield the title compound (2 g). The mother liquors were then purified on silica (gradient elution, 1-8% acetone/DCM) to yield an oil, which was further purified by trituration with Et₂O to yield more title compound as a white solid (1 g). LRMS (ESI) m/z 292 [(M+H)⁺; calculated for C₁₉H₁₈NO₂: 292].

### Step 3: 1-tert-butyl 2-methyl (2S,4R)-4-{[4-(benzyloxy)-3-(prop-2-en-1-yl)quinolin-2-yl]oxy}pyrrolidine-, 2-dicarboxylate

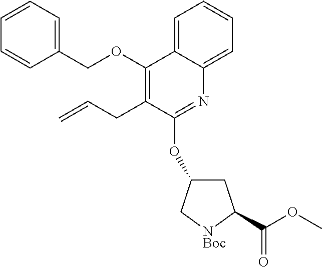 [SMILES: C=CCc1c(O[C@@H]2C[C@@H](C(=O)OC)N(C(=O)OC(C)(C)C)C2)nc2ccccc2c1OCc1ccccc1]

To a mixture 1-t-butyl 2-methyl (2S,4S)-4-{[(4-bromophenyl)sulfonyl]oxy}pyrrolidine-1,2-dicarboxylate (Intermediate A5) (5 g, 10.95 mmol) and the product from step 2 (2.9 g, 9.95 mmol) in N-Methyl-2-pyrrolidinone (25 mL), cesium carbonate (9.7 g, 29.9 mmol) was added, and the mixture was stirred at 60° C. for 1 hour. Additional Intermediate A5 (1 g) and cesium carbonate (1 g) were then added, and heating was continued for 3 hours. Additional Intermediate A5 (1 g) and cesium carbonate added (1 g) were then added and heating was continued overnight. The reaction was then cooled and quenched into water (150 mL), and aq. KHSO₄ was added to pH=3.5. The mixtures was then extracted with EtOAc (150 mL), and the organic layer was washed with aq. NaHCO₃ and brine, dried over sodium sulfate, filtered concentrated in vacuo. The residue was purified on silica (gradient elution, 10-30% ethyl acetate/hexanes) to give impure title compound. Further purification was then carried out via reverse phase column chromatography (gradient elution, 10-100% acetonitrile/0.15% TFA-water) to yield pure title compound (3 g). LRMS (ESI) m/z 519 [(M+H)⁺; calculated for C₃₀H₃₅N₂O₆: 519].

### Step 4: methyl (4R)-4-{[4-(benzyloxy)-3-(prop-2-en-1-yl)quinolin-2-yl]oxy}-L-prolinate

The title compound was prepared according to the procedure for Intermediate D5, step 6. LRMS (ESI) m/z 419 [(M+H)⁺; calcd for C₂₅H₂₇N₂O₄: 419].

### Example 1

### (1R,18R,20R,24S,27S)-24-Cyclopentyl-6-methoxy-20-methyl-N-[(1R,2S)-1-({[(1-methylcyclopropyl)sulfonyl]amino}carbonyl)-2-vinylcyclopropyl]-22,25-dioxo-2,21-dioxa-4,11,23,26-tetraazapentacyclo[24.2.1.0^{3,12}.0^{5,10}.0^{18,20}]nonacosa-3,5,7,9,11-pentaene-27-carboxamide

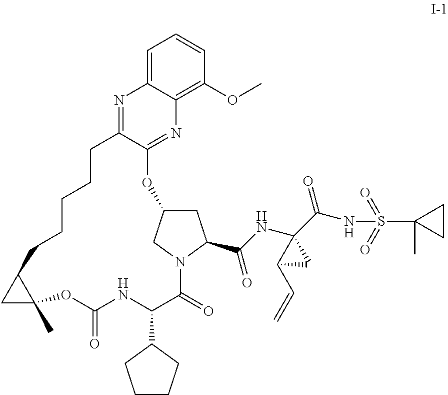 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCc1nc3cccc(OC)c3nc1O2)C(=O)NS(=O)(=O)C1(C)CC1]

### Step 1: Methyl (2S,4R)-1-{(2S)-2-[({[(1R,2R))-2-allyl-1-methylcyclopropyl]oxy}carbonyl)amino]-2-cyclopentylacetyl}-4-[(3-but-3-en-1-yl-8-methoxyquinoxalin-2-yl)oxy]pyrrolidine-2-carboxylate and Methyl (2S,4R)-1-{(2S)-2-[({[(1S,2S))-2-allyl-1-methylcyclopropyl]oxy}carbonyl)amino]-2-cyclopentylacetyl}-4-[(3-but-3-en-1-yl-8-methoxyquinoxalin-2-yl)oxy]pyrrolidine-2-carboxylate

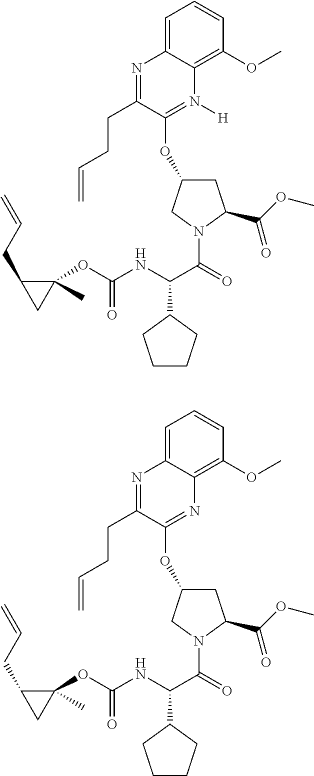

To a solution of intermediate D3 (9.5 g, 22.1 mmol) in DMF (145 ml), intermediate C1 (6.54 g, 23.2 mmol), HATU (8.83 g, 23.2 mmol), and DIEA (15.4 ml, 88.2 mmol) were added. After 1 hour, the reaction was diluted with EtOAc, and the organics were washed with 1N HCl (2×), brine, dried over Na₂ SO₄, filtered, and the solvent was removed in vacuo to yield an oil. The oil was purified by column chromatography on SiO₂ (gradient elution, 20-70% EtOAc/hexane) to give the title compound as a white foam from Et₂O (11.9 g). LRMS (ESI) m/z 621.2 [(M+H)⁺; calculated for C₃₄H₄₅N₄O₇: 621.3].

### Step 2: Methyl (1R,15E,18R,20R,24S,27S))-24-cyclopentyl-6-methoxy-20-methyl-22,25-dioxo-2,21-dioxa-4,11,23,26-tetraazapentacyclo[24.2.1.03,12.05,10.018,20]nonacosa-3,5,7,9,11,15-hexaene-27-carboxylate and Methyl (1R,15E,18S,20S,24S,27S))-24-cyclopentyl-6-methoxy-20-methyl-22,25-dioxo-2,21-dioxa-4,11,23,26-tetraazapentacyclo[24.2.1.03,12.05,10.018,20]nonacosa-3,5,7,9,11,15-hexaene-27-carboxylate

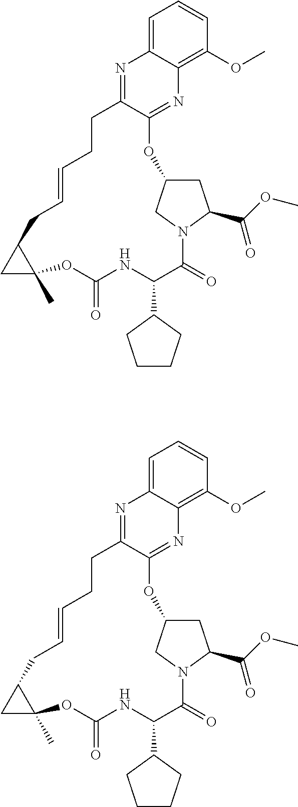 [SMILES: COC(=O)[C@@H]1C[C@@H]2CN1C(=O)[C@H](C1CCCC1)NC(=O)O[C@]1(C)C[C@H]1C/C=C/CCc1nc3cccc(OC)c3nc1O2 ; COC(=O)[C@@H]1C[C@@H]2CN1C(=O)[C@H](C1CCCC1)NC(=O)O[C@@]1(C)C[C@@H]1C/C=C/CCc1nc3cccc(OC)c3nc1O2]

To a degassed solution of product from step 1 (10.7 g, 17.24 mmol) in DCE (2.5 L), p-benzoquinone (0.373 g, 3.45 mmol) and Zhan 1B catalyst (1.200 g, 1.551 mmol) were added. The solution was warmed to 75° C. for 15 hours. The reaction was concentrated, and the residue was purified by column chromatography on SiO₂ (gradient elution, 20-60% EtOAc/hexane) to give the title compound as a brown foam from Et₂O (9.0 g). LRMS (ESI) m/z 593.2 [(M+H)⁺; calculated for C₃₂H₄₁N4O₇: 593.3].

### Step 3: methyl (1R,18R,20R,24S,27S))-24-cyclopentyl-6-methoxy-20-methyl-22,25-dioxo-2,21-dioxa-4,11,23,26-tetraazapentacyclo[24.2.1.03,12.05,10.018,20]nonacosa-3,5,7,9,11-pentaene-27-carboxylate

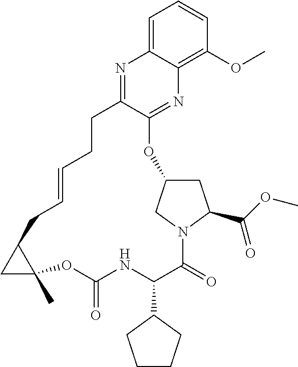 [SMILES: COC(=O)[C@@H]1C[C@@H]2CN1C(=O)[C@H](C1CCCC1)NC(=O)O[C@]1(C)C[C@H]1C/C=C/CCc1nc3cccc(OC)c3nc1O2]

To a degassed solution of product from step 2 (37.3 g, 62.9 mmol) in EtOH (800 mL), palladium, 5 wt % on calcium carbonate (2.68 g, 1.259 mmol), was added. The resulting slurry was placed under a hydrogen atmosphere (balloon) and allowed to stir for 1 hour. The reaction mixture was filtered through a pad of CELITE, and the cake was washed with EtOH followed by DCM. The filtrate was concentrated, and the resulting residue was purified by column chromatography on SiO₂ (gradient elution, 20-70% EtOAc/hexane) to give a yellow solid, which was a mixture of diastereomers. Purified by column chromatography on SiO₂ (isocratic elution, 5% acetone/CH₂Cl₂) to give the title compound as a white solid (15.6 g). The second diastereomer was then eluted with 10% acetone/DCM with 1% MeOH to yield methyl (1aS,5S,8S,10R,22aS)-5-cyclopentyl-13-methoxy-1a-methyl-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxylate (14.9 g), which was used in Example 2. LRMS (ESI) m/z 595.1 [(M+H)⁺; calculated for C₃₂H₄₃N₄O₇: 595.3].

### Step 4: (1R,18R,20R,24S,27S))-24-cyclopentyl-6-methoxy-20-methyl-22,25-dioxo-2,21-dioxa-4,11,23,26-tetraazapentacyclo[24.2.1.03,12.05,10.018,20]nonacosa-3,5,7,9,1-pentaene-27-carboxylic acid

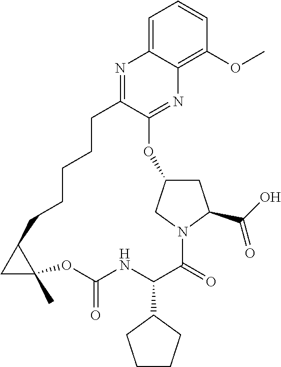 [SMILES: COc1cccc2nc3c(nc12)O[C@@H]1C[C@@H](C(=O)O)N(C1)C(=O)[C@H](C1CCCC1)NC(=O)O[C@]1(C)C[C@H]1CCCCC3]

To a solution of the product from step 3 (15.6 g, 26.2 mmol) in THF (200 mL)/water (100 mL), lithium hydroxide monohydrate (2.187 ml, 79 mmol) was added, and the mixture was stirred for 3 hours. The reaction mixture was treated with 6N HCl (13.2 mL), and the solvents were removed in vacuo. The resulting residue was partitioned between CH₂Cl₂ and KHSO₄ (10%) and extracted with CH₂Cl₂ (3×). The combined organics were washed with brine, dried over Na₂SO₄, and concentrated to yield the title compound as a tan solid from Et₂O (15.0 g). LRMS (ESI) m/z 581.1 [(M+H)⁺; calculated for C₃₁H₄₁N₄O₇: 581.3].

### Step 5: (1R,18R,20R,24S,27S))-24-cyclopentyl-6-methoxy-20-methyl-N-[(1R,2S)-1-({([(1-methylcyclopropyl)sulfonyl]amino}carbonyl)-2-vinylcyclopropyl]-22,25-dioxo-2,21-dioxa-4,11,23,26-tetraazapentacyclo[24.2.1.03,12.05,10.018,20]nonacosa-3,5,7,9,11-pentaene-27-carboxamide

To a solution of product from step 4 (5.8 g, 9.99 mmol) in DMF (60 mL), intermediate A4 (3.08 g, 10.99 mmol) and DIEA (6.98 ml, 40.0 mmol) were added, and then HATU (4.18 g, 10.99 mmol) was added while in an ice bath. The ice bath was removed, and the reaction was stirred for 1 hour. An additional amount of intermediate A4 (0.561 g, 2 mmol), DIEA (0.350 ml, 2 mmol), and HATU (0.760 g, 2 mmol) was added. After 1 hour, reaction was poured into water (270 mL) and stirred for 1 hour. The slurry was filtered, washed with water to yield a white solid. The solid was stirred in 30% EtOAc/hexane and warmed to 40° C. for 2 hours. It was then cooled to RT and stirred for 14 hours. The mixture was filtered to yield the title compound as a white solid (6.94 g). LRMS (ESI) m/z 807.2 [(M+H)⁺; calculated for C₄₁H₅₅N6O₉S: 807.3].

### Example 2

### (1aS,5S,8S,10R,22aS)-5-cyclopentyl-N-[(1R,2S)-2-ethenyl-1-{[(1-methylcyclopropyl sulfonyl]carbamoyl}cyclopropyl]-13-methoxy-1a-methyl-3,6-dioxo-1,1a,3,4,5,6,9,10,18,19,20,22,22a-tetradecahydro-8H-7,10-methanocyclopropa[18,19][1,10,3,6]dioxadiazacyclononadecino[11,12-b]quinoxaline-8-carboxamide

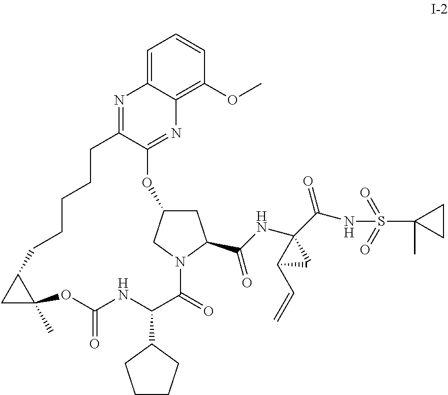 [SMILES: C=C[C@@H]1C[C@]1(NC(=O)[C@@H]1C[C@@H]2CN1C(=O)[C@H](C1CCCC1)NC(=O)O[C@@]1(C)C[C@@H]1CCCCCc1nc3cccc(OC)c3nc1O2)C(=O)NS(=O)(=O)C1(C)CC1]

The title compound was prepared following the procedures for Steps 4 and 5 from Example 1 using diastereomer 2 (methyl (1aS,5S,8S,10R,22aS)-5-cyclopentyl-13-methoxy-1a-methyl-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxylate) isolated from Step 3, Example 1. LRMS (ESI) m/z 807.2 [(M+H)⁺; calculated for C₄₁H₅₅N₆O₉S: 807.3].

### Example 3

### (1R,18R,22R,26S,29S)-26-Cyclopentyl-N-((1R,2S)-1-{[(cyclopropylsulfonyl)amino]carbonyl}-2-vinylcyclopropyl)-7-methoxy-22-methyl-24,27-dioxo-2,23-dioxa-11,25,28-triazapentacyclo[26.2.1.0^{3,12}.0^{5,10}.0^{18,22}]hentriaconta-3(12),4,5,7,9,10-hexaene-29-carboxamide (WO 2008/057209, Example 116)

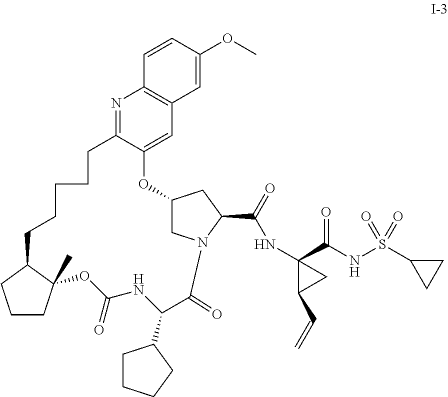 [SMILES: C=C[C@@H]1C[C@]1(NC(=O)[C@@H]1C[C@@H]2CN1C(=O)[C@H](C1CCCC1)NC(=O)O[C@]1(C)CCC[C@H]1CCCCCc1nc3ccc(OC)cc3cc1O2)C(=O)NS(=O)(=O)C1CC1]

### Step 1: methyl (4R)-4-[(2-bromo-6-methoxyquinolin-3-yl)oxy]-1-{(2S)-2-cyclopentyl-2-[({[(1R,2S)-1-methyl-2-(pent-4-en-1-yl)cyclopentyl]oxy}carbonyl)amino]acetyl}-L-prolinate and methyl (4R)-4-[(2-bromo-6-methoxyquinolin-3-yl)oxy]-1-{(2S)-2-cyclopentyl-2-[({[(1S,2R)-1-methyl-2-(pent-4-en-1-yl)cyclopentyl]oxy}carbonyl)amino]acetyl}-L-prolinate

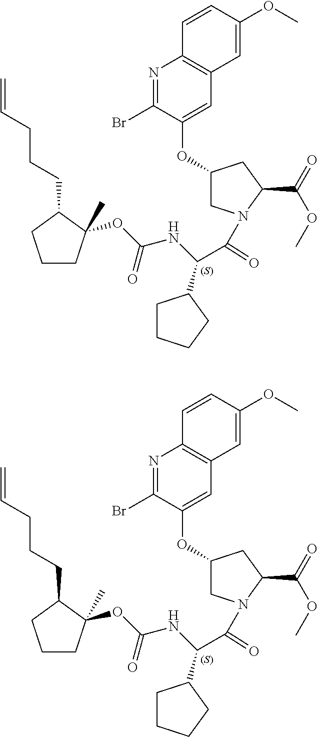

The title compound was prepared according to Example 1, Step 1 using Intermediates C6 and D1. LRMS (ESI) m/z 700/702 [(M+H)⁺; calculated for C₃₅H₄₇BrN₃O₇: 700/702].

### Step 2: methyl (4R)-1-{(2S)-2-cyclopentyl-2-[({[(1R,2R)-1-methyl-2-(pent-4-en-1-yl)cyclopentyl]oxy}carbonyl)amino]acetyl}-4-[(2-ethenyl-6-methoxyquinolin-3-yl)oxy]-L-prolinate and methyl (4R)-1-{(2S)-2-cyclopentyl-2-[({[(1S,2R)-1-methyl-2-(pent-4-en-1-yl)cyclopentyl]oxy}carbonyl)amino]acetyl}-4-[(2-ethenyl-6-methoxyquinolin-3-yl)oxy]-L-prolinate

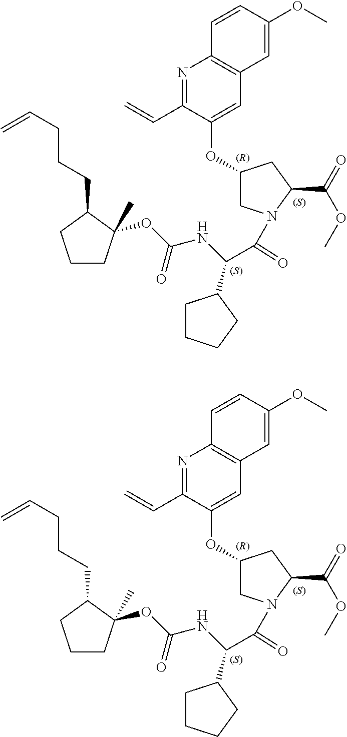

To a solution of the product from step 1 (735 mg, 1.0 mmol) in EtOH (70 mL), potassium vinyltrifluoroborate (211 mg, 1.5 mmol), triethylamine (0.22 mL, 1.5 mmol), and [1,1-Bis(diphenylphosphino)ferrocene]dichloropalladium(II)-CH₂Cl₂ complex (25 mg, 0.03 mmol) were added. The reaction was heated to reflux for 1 hour, the solvent was removed in vacuo, and the residue was taken up in EtOAc and washed with water. The organic layer was dried over Na₂SO₄, and the solvent was removed in vacuo. The material was purified on silica (gradient elution, 10-40% EtOAc/hexanes) to yield the title compound (402 mg) as a yellow oil. LRMS (ESI) m/z 648 [(M+H)⁺; calculated for C₃₇H₅₀N₃O₇: 648].

### Step 3: (1R,18R,22R,26S,29S)-26-Cyclopentyl-N-((1R,2S)-1-{[(cyclopropylsulfonyl)amino]carbonyl}-2-vinylcyclopropyl)-7-methoxy-22-methyl-24,27-dioxo-2,23-dioxa-11,25,28-triazapentacyclo[26.2.1.0^{3,12}0^{5,10}.0^{18,22}]hentriaconta-3 (12), 4,5,7,9,10-hexaene-29-carboxamide

The title compound was prepared according to the procedures in Example 1, steps 2-5 using Intermediate A1 and with separation of the two diastereomers following step 5 via reverse phase chromatography. LRMS (ESI) m/z 820 [(M+H)⁺; calculated for C₄₃H₅₈N5O₉S: 820].

### Example 4

### (1R,14E,18R,20R,24S,27S)-24-Cyclopentyl-N-((1R,2S)-1-{[(cyclopropylsulfonyl)amino]carbonyl}-2-vinylcyclopropyl)-11-hydroxy-20-methyl-22,25-dioxo-2,21-dioxa-4,23,26-triazapentacyclo[24.2.1.0^{3,12}.0^{5,10}.0^{18,20}]nonacosa-3,5,7,9,11,14-hexaene-27-carboxamide

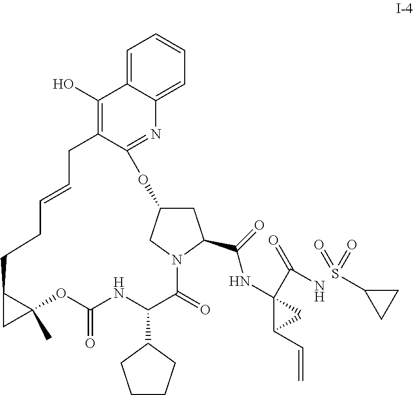

### Step 1: methyl (1aR,5S,8S,10R,19E,22aR)-17-(benzyloxy)-5-cyclopentyl-1a-methyl-3,6-dioxo-1,1a,3,4,5,6,9,10,18,21,22,22a-dodecahydro-8H-70-m-methanocyclopropa[18,19][1,10,3,6]dioxadiazacyclononadecino[11,12-b]quinoline-8-carboxylate and methyl (1aS,5S,8S,10R,19E,22aS)-17-(benzyloxy)-5-cyclopentyl-1a-methyl-3,6-dioxo-1,1a,3,4,5,6,9,10,18,21,22,22a-dodecahydro-8H-7,10-methanocyclopropa[18,19][1,10,3,6]dioxadiazacyclononadecino[11,12-b]quinoline-8-carboxylate

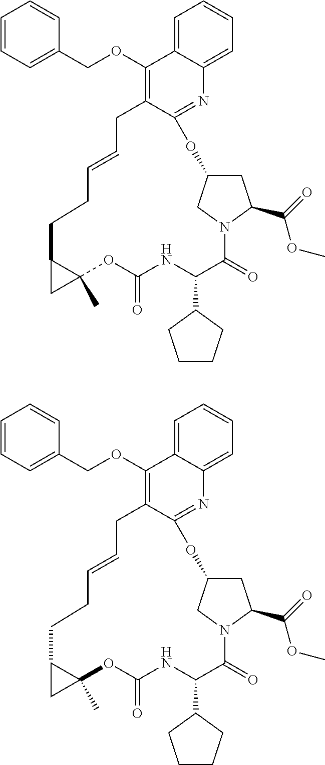

The title compounds were prepared according to Example 1, steps 1-2 using Intermediates C2 and D7. The crude products from step 2 were purified and separated on silica (gradient elution, 5-50% EtOAc/hexanes) to yield methyl (1aR,5S,8S,100R,19E,22aR)-17-(benzyloxy)-5-cyclopentyl-1a-methyl-3,6-dioxo-1,1a,3,4,5,6,9,10,18,21,22,22a-dodecahydro-8H-7,10-methanocyclopropa[18,19][1,10,3,6]dioxadiazacyclononadecino[11,12-b]quinoline-8-carboxylate as the first eluting isomer and methyl (1aS,5S,8S,10R,19E,22aS)-17-(benzyloxy)-5-cyclopentyl-1a-methyl-3,6-dioxo-1,1a,3,4,5,6,9,10,18,21,22,22a-dodecahydro-8H-7,10-methanocyclopropa[18,19][1,10,3,6]dioxadiazacyclononadecino[11,12-b]quinoline-8-carboxylate as the second eluting isomer. LRMS (ESI) m/z 669 [(M+H)⁺; calculated for C₃₉H₄₆N₃O₇S: 669].

### Step 2: (1aR,5S,8S,10R,19E,22aR)-17-(benzyloxy)-5-cyclopentyl-N-{(1R,2S)-1-[(cyclopropylsulfonyl)carbamoyl]-2-ethenylcyclopropyl}-1a-methyl-3,6-dioxo-1,1a,3,4,5,6,9,10,18,21,22,22a-dodecahydro-8H-7,10-methanocyclopropa[18,19][1,10,3,6]dioxadiazacyclononadecino[11,12-b]quinoline-8-carboxamide and

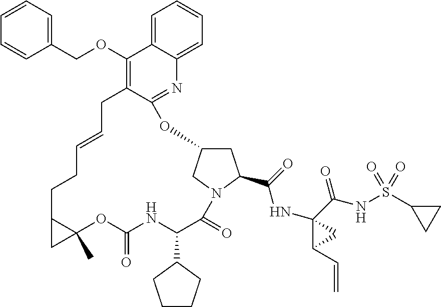 [SMILES: C=C[C@@H]1C[C@]1(NC(=O)[C@@H]1C[C@@H]2CN1C(=O)[C@H](C1CCCC1)NC(=O)O[C@]1(C)CC1CC/C=C/Cc1c(nc3ccccc3c1OCc1ccccc1)O2)C(=O)NS(=O)(=O)C1CC1]

Using the first eluting isomer from step 1, the title compound was prepared according to example 1, steps 4-5 using Intermediate A1. LRMS (ESI) m/z 867 [(M+H)⁺; calculated for C₄₇H₅₆N₅O₉S: 867].

### Step 3: (1R,14E,18R,20R,24S,27S)-24-Cyclopentyl-N-((1R,2S)-1-{[(cyclopropylsulfonyl)amino]carbonyl}-2-vinylcyclopropyl)-11-hydroxy-20-methyl-22,25-dioxo-2,21-dioxa-4,23,26-triazapentacyclo[24.2.1.0^{3,12}0^{5,10}0^{18,20}]nonacosa-3,5,7,9,11,14-hexaene-27-carboxamide

To the product from step 2 (51 mg, 0.059 mmol), TFA (681 μl, 8.83 mmol) was added, and the mixture was warmed to 55° C. After 45 minutes, the reaction was concentrated without heat, and the residue was dissolved in ACN and purified by reverse phase chromatography. Fractions were worked up with EtOAc and saturated aqueous NaHCO₃. The organic layer was dried over Na₂SO₄, and the solvent was removed in vacuo to yield the title compound (17 mg). LRMS (ESI) m/z 776 [(M+H)⁺; calculated for C₄₀H₅₀N₅O₉S: 776].

### Example 5

### (1aR,5S8S,10R,19E,23aR)-5-tert-butyl-N-{(1R,2S)-1-[(cyclopropylsulfonyl)carbamoyl]-2-ethenylcyclopropyl}-1a-methyl-3,6,12-trioxo-1a,3,4,5,6,9,10,14,21,22,23,23a-dodecahydro-1H,8H-7,10:13,15-dimethanocycloproa[n][4,13,2,8,11]benzodioxatriazacyclodocosine-8(12H)-carboxamide

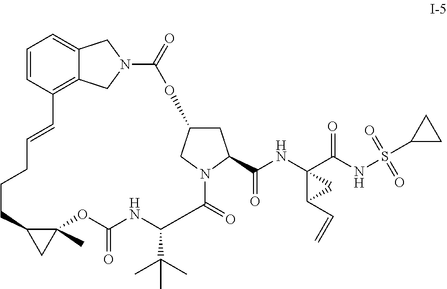 [SMILES: C=C[C@@H]1C[C@]1(NC(=O)[C@@H]1C[C@@H]2CN1C(=O)[C@H](C(C)(C)C)NC(=O)O[C@]1(C)C[C@H]1CCC/C=C/c1cccc3c1CN(C3)C(=O)O2)C(=O)NS(=O)(=O)C1CC1]

The title compound was prepared according to example 1, steps 1, 2, 4, and 5 using intermediates A1, C4-1, and D4. LRMS (ESI) m/z 766 [(M+H)⁺; calculated for C₃₉H₅₂N₅O₉S: 766].

### Examples 6-32

By using the appropriate procedures and the appropriate A, C, and D intermediates, the compounds of Examples 6-32 were prepared, as shown in Table 1 below. All compounds prepared from racemic intermediates C1, C2, C3, C4, C5, C6, and C7 were separated from resulting diastereomers using reverse or normal phase column chromatography following macrocycle formation or after the final step.

| | | | LRMS | Inter- | Proced- |
|---|---|---|---|---|---|
| Ex. | Structure | Name | (M + H)⁺ | mediates | ure(s) |
| 6 | 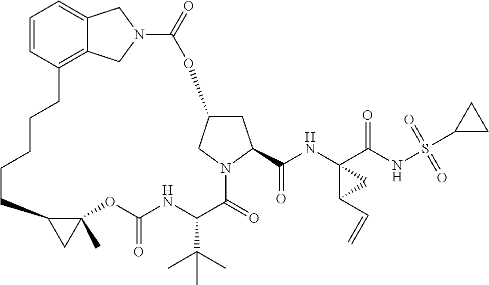 [SMILES: C=C[C@@H]1C[C@]1(NC(=O)[C@@H]1C[C@@H]2CN1C(=O)[C@H](C(C)(C)C)NC(=O)O[C@]1(C)C[C@H]1CCCCCc1cccc3c1CN(C3)C(=O)O2)C(=O)NS(=O)(=O)C1CC1] I-6 | (1aR,5S,8S,10R,23aR)- 5-tert-butyl-N- {(1R,2S)-1- [(cyclopropylsulfonyl) carbamoyl]-2- ethenylcyclopropyl}- 1a-methyl-3,6,12- trioxo-1a,3,4,5,6,9,10, 14,19,20,21,22,23,23a- tetradecahydro-1H,8H- 7,10:13,15-dimethano cyclopropa [n][4,13,2, 8,11] benzodioxatriaza cyclodocosine-8(12H)- carboxamide | 768 | A1, C4-1, D4 | Exam- ple 1 |
| 7 | 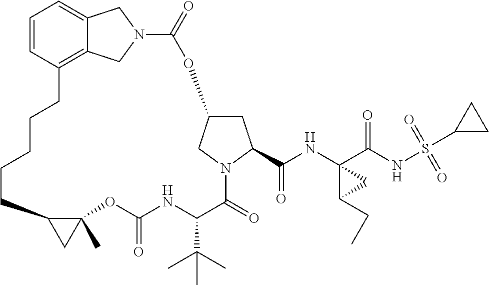 [SMILES: CC[C@@H]1C[C@]1(NC(=O)[C@@H]1C[C@@H]2CN1C(=O)[C@H](C(C)(C)C)NC(=O)O[C@]1(C)C[C@H]1CCCCCc1cccc3c1CN(C3)C(=O)O2)C(=O)NS(=O)(=O)C1CC1] I-7 | (1aR,5S,8S,10R,23aR)- 5-tert-butyl-N- {(1R,2R)-1- [(cyclopropylsulfonyl) carbamoyl]-2- ethylcyclopropyl}-1a- methyl-3,6,12-trioxo- 1a,3,4,5,6,9,10,14,19, 20,21,22,23,23a- tetradecahydro-1H,8H- 7,10:13,15-dimethano cyclopropa[n][4,13,2,8, 11]benzodioxatriaza cyclodocosine-8(12H)- carboxamide | 770 | A3, C4-1, D4 | Exam- ple 1 |
| 8 | 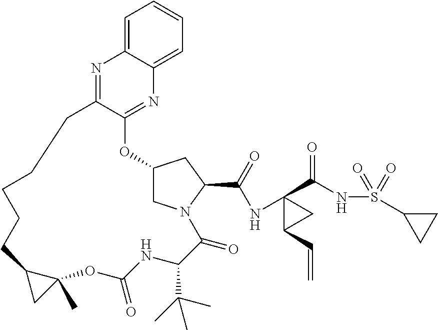 [SMILES: C=C[C@@H]1C[C@]1(NC(=O)[C@@H]1C[C@@H]2CN1C(=O)[C@H](C(C)(C)C)NC(=O)O[C@]1(C)C[C@H]1CCCCCc1nc3ccccc3nc1O2)C(=O)NS(=O)(=O)C1CC1] I-8 | (1aR,5S,8S,10R,22aR)- 5-tert-butyl-N- {(1R,2S)-1- [(cyclopropylsulfonyl) carbamoyl]-2- ethenylcyclopropyl}- 1a-methyl-3,6-dioxo- 1,1a,3,4,5,6,9,10,18,19, 20,21,22,22a-tetra decahydro-8H-7,10- methanocyclopropa[18, 19][1,10,3,6]dioxatriaza cyclononadecino[11,12- b]quinoxaline-8- carboxamide | 751 | A1, C4, D2 | Exam- ple 3 |
| 9 | 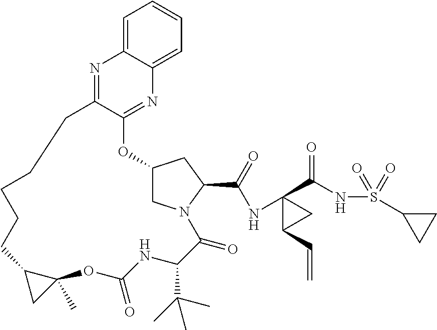 [SMILES: C=C[C@@H]1C[C@]1(NC(=O)[C@@H]1C[C@@H]2CN1C(=O)[C@H](C(C)(C)C)NC(=O)O[C@@]1(C)C[C@@H]1CCCCCc1nc3ccccc3nc1O2)C(=O)NS(=O)(=O)C1CC1] I-9 | (1aS,5S,8S,10R,22aS)- 5-tert-butyl-N- {(1R,2S)-1- [(cyclopropylsulfonyl) carbamoyl]-2- ethenylcyclopropyl}- 1a-methyl-3,6-dioxo- 1,1a,3,4,5,6,9,10,18,19, 20,21,22,22a-tetra decahydro-8H-7,10- methanocyclopropa[18, 19][1,10,3,6]dioxadiaza cyclononadecino[11,12- b]quinoxaline-8- carboxamide | 751 | A1, C4, D2 | Exam- ple 3 |
| 10 | 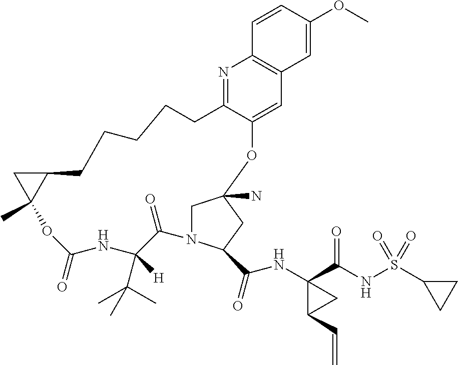 [SMILES: C=C[C@@H]1C[C@]1(NC(=O)[C@@H]1C[C@]2(N)CN1C(=O)[C@H](C(C)(C)C)NC(=O)O[C@]1(C)C[C@H]1CCCCCc1nc3ccc(OC)cc3cc1O2)C(=O)NS(=O)(=O)C1CC1] I-10 | (1aR,5S,8S,10R,22aR)- 5-tert-butyl-N- {(1R,2S)-1- [(cyclopropylsulfonyl) carbamoyl]-2- ethenylcyclopropyl}- 14-methoxy-1a-methyl- 3,6-dioxo-1,1a,3,4,5, 6,9,10,18,19,20,21,22, 22a-tetradecahydro-8H- 7,10-methanocyclo propa[18,19][1,10,3,6] dioxadiazacyclonona decino[12,11-b] quinoline-8- carboxamide | 780 | A3, C4, D1 | Exam- ple 3 |
| 11 | 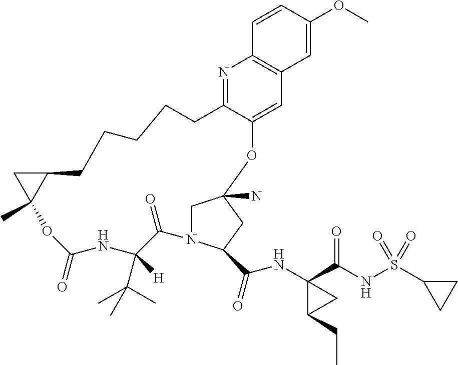 [SMILES: CC[C@@H]1C[C@]1(NC(=O)[C@@H]1C[C@]2(N)CN1C(=O)[C@H](C(C)(C)C)NC(=O)O[C@]1(C)C[C@H]1CCCCCc1nc3ccc(OC)cc3cc1O2)C(=O)NS(=O)(=O)C1CC1] I-11 | (1aR,5S,8S,10R,22aR)- 5-tert-butyl-N- {(1R,2R)-1- [(cyclopropylsulfonyl) carbamoyl]-2- ethylcarbamoyl]-2- ethylcyclopropyl}-14- methoxy-1a-methyl- 3,6-dioxo-1,1a,3,4,5,6, 9,10,18,19,20,21,22,22a- tetradecahydro-8H- 7,10-methanocyclo propa[18,19][1,10,3,6] dioxadiazacyclonona decino[12,11-b] quinoline-8- carboxamide | 782 | A3, C4, D1 | Exam- ple 3 |
| 12 | 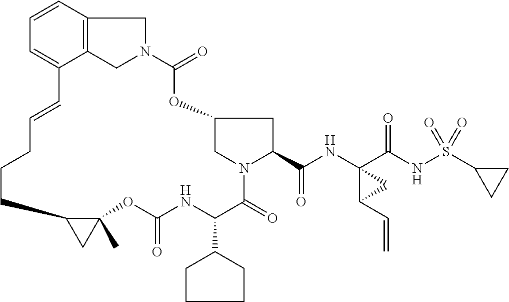 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCC/C=C/c1cccc3c1CN(C3)C(=O)O2)C(=O)NS(=O)(=O)C1CC1] I-12 | (1aR,5S,8S,10R,19E,23aR)- 5-cyclopentyl-N- {(1R,2S)-1- [(cyclopropylsulfonyl) carbamoyl]-2- ethenylcyclopropyl}- 1a-methyl-3,6,12- trioxo-1a,3,4,5,6,9,10, 14,21,22,23,23a- dodecahydro-1H,8H- 7,10:13,15-dimethano cyclopropa[n][4,13,2,8, 11]benzodioxatriaza cyclodocosine-8(12H)- carboxamide | 778 | A1, C3-1, D4 | Exam- ple 5 |
| 13 | 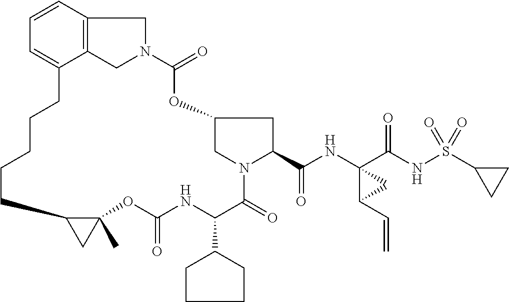 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCc1cccc3c1CN(C3)C(=O)O2)C(=O)NS(=O)(=O)C1CC1] I-13 | (1aR,5S,8S,10R,23aR)- 5-cyclopentyl-N- {(1R,2S)-1- [(cyclopropylsulfonyl) carbamoyl]-2- ethenylcyclopropyl}- 1a-methyl-3,6,12- trioxo-1a,3,4,5,6,9,10, 14,19,20,21,22,23,23a- tetradecahydro-1H,8H- 7,10:13,15-dimethano cyclopropa[n][4,13,2,8, 11]benzodioxatriaza cyclodocosine-8(12H)- carboxamide | 780 | A1, C3-1, D4 | Exam- ple 1 |
| 14 | 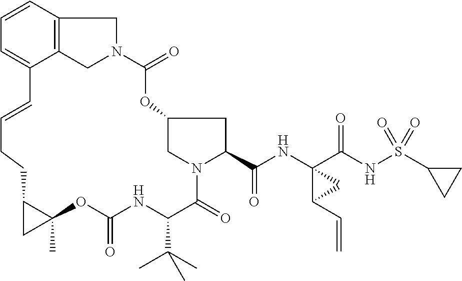 [SMILES: C=C[C@@H]1C[C@]1(NC(=O)[C@@H]1C[C@@H]2CN1C(=O)[C@H](C(C)(C)C)NC(=O)O[C@@]1(C)C[C@@H]1CC/C=C/c1cccc3c1CN(C3)C(=O)O2)C(=O)NS(=O)(=O)C1CC1] I-14 | (1aS,5S,8S,10R,19E,22aS)- 5-tert-butyl-N- {(1R,2S)-1- [(cyclopropylsulfonyl) carbamoyl]-2- ethenylcyclopropyl}- 1a-methyl-3,6,12- trioxo-1,1a,3,4,5,6,9, 10,14,21,22,22a- dodecahydro-8H- 7,10:13,15-dimethano cyclopropa[n][4,13,2,8, 11]benzodioxatriaza cyclohenicosine- 8(12H)-carboxamide | 752 | A1, C7, D4 | Exam- ple 5 |
| 15 | 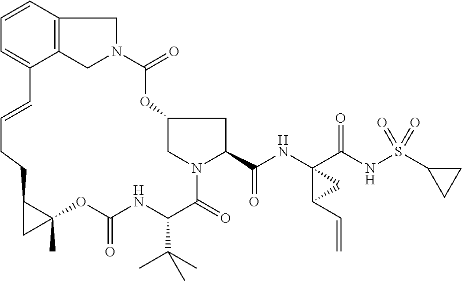 [SMILES: C=C[C@@H]1C[C@]1(NC(=O)[C@@H]1C[C@@H]2CN1C(=O)[C@H](C(C)(C)C)NC(=O)O[C@]1(C)C[C@H]1CC/C=C/c1cccc3c1CN(C3)C(=O)O2)C(=O)NS(=O)(=O)C1CC1] I-15 | (1aR,5S,8S,10R,19E,22aR)- 5-tert-butyl-N- {(1R,2S)-1- [(cyclopropylsulfonyl) carbamoyl]-2- ethenylcyclopropyl}- 1a-methyl-3,6,12- trioxo-1,1a,3,4,5,6,9, 10,14,21,22,22a- dodecahydro-8H-7,10: 13,15-dimethanocyclo propa[n][4,13,2,8,11] benzodioxatriazacyclo henicosine-8(12H)- carboxamide | 752 | A1, C7, D4 | Exam- ple 5 |
| 16 | 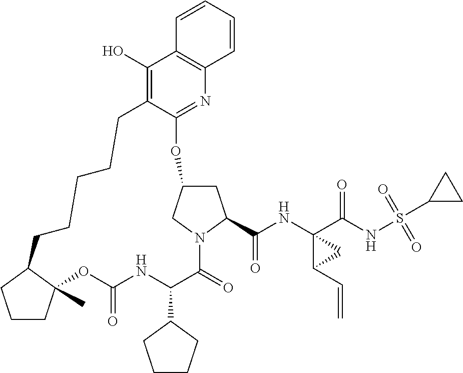 [SMILES: C=C[C@@H]1C[C@]1(NC(=O)[C@@H]1C[C@@H]2CN1C(=O)[C@H](C1CCCC1)NC(=O)O[C@]1(C)CCC[C@H]1CCCCCc1c(nc3ccccc3c1O)O2)C(=O)NS(=O)(=O)C1CC1] I-16 | (3aR,7S,10S,12R,24aR)- 7-cyclopentyl-N- {(1R,2S)-1- [(cyclopropylsulfonyl) carbamoyl]-2- ethenylcyclopropyl}- 19-hydroxy-3a-methyl- 5,8-dioxo-1,2,3,3a,5,6, 7,8,11,12,20,21,22,23,24, 24a-hexadecahydro- 10H-9,12-methano cyclopenta[18,19][1,10, 3,6]dioxadiazacyclo nonadecino[11,12- b]quinoline-10- carboxamide | 806 | A1, C5, D7 | Exam- ple 1 |
| 17 | 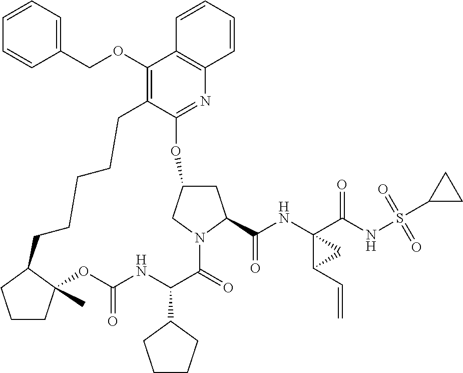 [SMILES: C=C[C@@H]1C[C@]1(NC(=O)[C@@H]1C[C@@H]2CN1C(=O)[C@H](C1CCCC1)NC(=O)O[C@]1(C)CCC[C@H]1CCCCCc1c(nc3ccccc3c1OCc1ccccc1)O2)C(=O)NS(=O)(=O)C1CC1] I-17 | (3aR,7S,10S,12R,21E, 24aS)-19-(benzyloxy)-7- cyclopentyl-N- {(1R,2S)-1- [(cyclopropylsulfonyl) carbamoyl]-2- ethenylcyclopropyl}- 3a-methyl-5,8-dioxo- 1,2,3,3a,5,6,7,8,11,12, 20,23,24,24a-tetradeca hydro-10H-9,12- methanocyclopenta[18, 19][1,10,3,6]dioxadiaza cyclononadecino[11,12- b]quinoline-10- carboxamide | 894 | A1, C5, D7 | Exam- ple 5 |
| 18 | 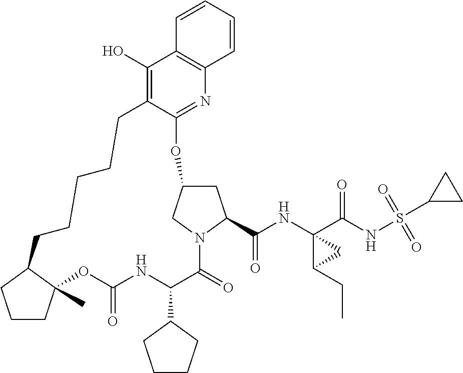 [SMILES: CC[C@@H]1C[C@]1(NC(=O)[C@@H]1C[C@@H]2CN1C(=O)[C@H](C1CCCC1)NC(=O)O[C@]1(C)CCC[C@H]1CCCCCc1c(nc3ccccc3c1O)O2)C(=O)NS(=O)(=O)C1CC1] I-18 | (3aR,7S,10S,12R,24aR)- 7-cyclopentyl-N- {(1R,2R)-1- [(cyclopropylsulfonyl) carbamoyl]-2- ethylcyclopropyl}-19- hydroxy-3a-methyl-5,8- dioxo-1,2,3,3a,5,6,7, 8,11,12,20,21,22,23,24, 24a-hexadecahydro- 10H-9,12-methano cyclopenta[18,19][1,10, 3,6]dioxadiazacyclo nonadecino[11,12- b]quinoline-10- carboxamide | 808 | A3, C5, D7 | Exam- ple 1 |
| 19 | 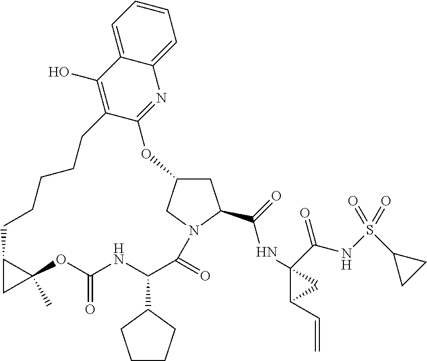 [SMILES: C=C[C@@H]1C[C@]1(NC(=O)[C@@H]1C[C@@H]2CN1C(=O)[C@H](C1CCCC1)NC(=O)O[C@@]1(C)C[C@@H]1CCCCCc1c(nc3ccccc3c1O)O2)C(=O)NS(=O)(=O)C1CC1] I-19 | (1aS,5S,8S,10R,19E,22aS)- 5-cyclopentyl-N- {(1R,2S)-1- [(cyclopropylsulfonyl) carbamoyl]-2- ethenylcyclopropyl}- 17-hydroxy-1a-methyl- 3,6-dioxo-1,1a,3,4,5,6, 9,10,18,21,22,22a- dodecahydro-8H-7,10- methanocyclopropa[18, 19][1,10,3,6]dioxadiaza cyclononadecino[11,12- b]quinolin-8- carboxamide | 776 | A1, C2, D7 | Exam- ple 4 |
| 20 | 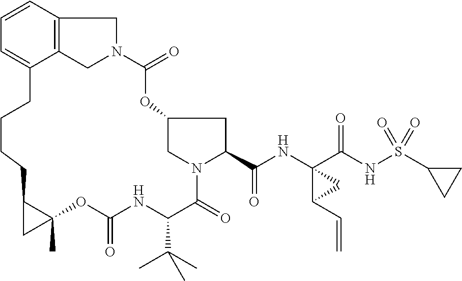 [SMILES: C=C[C@@H]1C[C@]1(NC(=O)[C@@H]1C[C@@H]2CN1C(=O)[C@H](C(C)(C)C)NC(=O)O[C@]1(C)C[C@H]1CCCCc1cccc3c1CN(C3)C(=O)O2)C(=O)NS(=O)(=O)C1CC1] I-20 | (1aR,5S,8S,10R,22aR)- 5-tert-butyl-N- {(1R,2S)-1- [(cyclopropylsulfonyl) carbamoyl]-2- ethenylcyclopropyl}- 1a-methyl-3,6,12- trioxo-1,1a,3,4,5,6,9, 10,14,19,20,21,22,22a- tetradecahydro-8H- 7,10:13,15-dimethano cyclopropa[n][4,13,2,8, 11]benzodioxatriaza cyclohenicosine- 8(12H)-carboxamide | 754 | A1, C7, D4 | Exam- ple 1 |
| 21 | 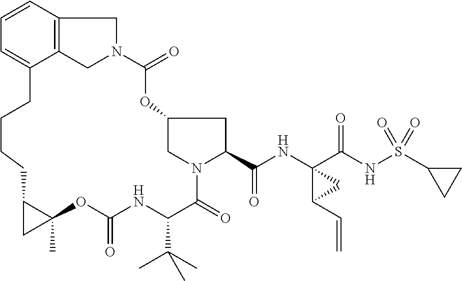 [SMILES: C=C[C@@H]1C[C@]1(NC(=O)[C@@H]1C[C@@H]2CN1C(=O)[C@H](C(C)(C)C)NC(=O)O[C@@]1(C)C[C@@H]1CCCCc1cccc3c1CN(C3)C(=O)O2)C(=O)NS(=O)(=O)C1CC1] I-21 | (1aS,5S,8S,10R,22aS)- 5-tert-butyl-N- {(1R,2S)-1- [(cyclopropylsulfonyl) carbamoyl]-2- ethenylcyclopropyl}- 1a-methyl-3,6,12- trioxo-1,1a,3,4,5,6, 9,10,14,19,20,21,22,22a- tetradecahydro-8H- 7,10:13,15-dimethano cyclopropa[n][4,13,2,8, 11]benzodioxatriaza cyclohenicosine- 8(12H)-carboxamide | 754 | A1, C7, D4 | Exam- ple 1 |
| 22 | 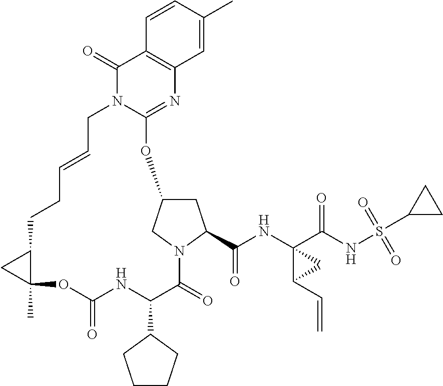 [SMILES: C=C[C@@H]1C[C@]1(NC(=O)[C@@H]1C[C@@H]2CN1C(=O)[C@H](C1CCCC1)NC(=O)O[C@@]1(C)C[C@@H]1CC/C=C/Cn1c(nc3cc(C)ccc3c1=O)O2)C(=O)NS(=O)(=O)C1CC1] I-22 | (1aS,5S,8S,10R,20E,23aS)- 5-cyclopentyl-N- {(1R,2S)-1- [(cyclopropylsulfonyl) carbamoyl]-2- ethenylcyclopropyl}- 1a,14-dimethyl-3,6,17- trioxo-1,1a,3,4,5,6, 9,10,19,22,23,23a- dodecahydro-8H,17H- 7,10-methano cyclopropa[18,19] [1,10,3,6,12]dioxatriaza cyclononadecino[11,12- b]quinazoline-8- carboxamide | 791 | A1, C2, D6 | Exam- ple 5 |
| 23 | 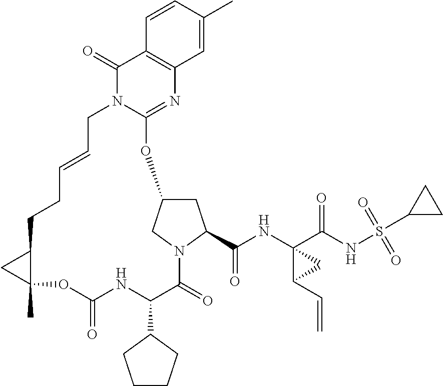 [SMILES: C=C[C@@H]1C[C@]1(NC(=O)[C@@H]1C[C@@H]2CN1C(=O)[C@H](C1CCCC1)NC(=O)O[C@]1(C)C[C@H]1CC/C=C/Cn1c(nc3cc(C)ccc3c1=O)O2)C(=O)NS(=O)(=O)C1CC1] I-23 | (1aR,5S,8S,10R,20E,23aR)- 5-cyclopentyl-N- {(1R,2S)-1- [(cyclopropylsulfonyl) carbamoyl]-2- ethenylcyclopropyl}- 1a,14-dimethyl-3,6,17- trioxo-1,1a,3,4,5,6, 9,10,19,22,23,23a- dodecahydro-8H,17H- 7,10-methano cyclopropa[18,19][1, 10,3,6,12]dioxatriaza cyclononadecino[11,12- b]quinazoline-8- carboxamide | 791 | A1, C2, D6 | Exam- ple 5 |
| 24 | 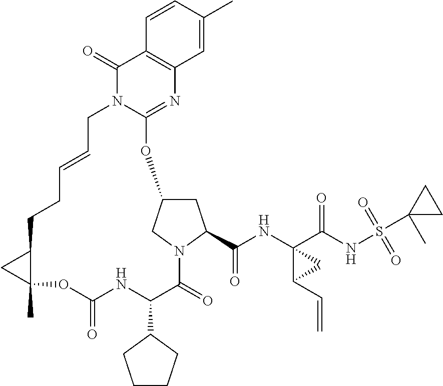 [SMILES: C=C[C@@H]1C[C@]1(NC(=O)[C@@H]1C[C@@H]2CN1C(=O)[C@H](C1CCCC1)NC(=O)O[C@]1(C)C[C@H]1CC/C=C/Cn1c(nc3cc(C)ccc3c1=O)O2)C(=O)NS(=O)(=O)C1(C)CC1] I-24 | (1aR,5S,8S,10R,20E,23aR)- 5-cyclopentyl-N- [(1R,2S)-2-ethenyl-1- {[(1-methylcyclo propyl)sulfonyl] carbamoyl}cyclopropyl]- 1a,14-dimethyl- 3,6,17-trioxo-1,1a,3, 4,5,6,9,10,19,22,23,23a- dodecahydro-8H,17H- 7,10-methanocyclo propa[18,19][1,10,3,6,12] dioxatriazacyclo nonadecino[11,12- b]quinazoline-8- carboxamide | 805 | A4, C2, D6 | Exam- ple 5 |
| 25 | 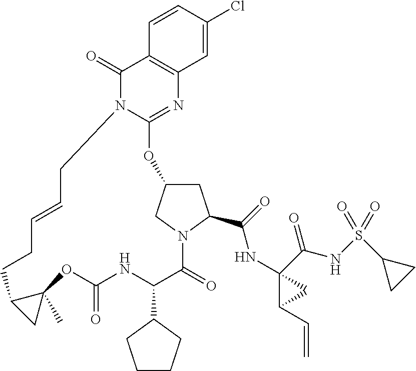 [SMILES: C=C[C@@H]1C[C@]1(NC(=O)[C@@H]1C[C@@H]2CN1C(=O)[C@H](C1CCCC1)NC(=O)O[C@@]1(C)C[C@@H]1CC/C=C/Cn1c(nc3cc(Cl)ccc3c1=O)O2)C(=O)NS(=O)(=O)C1CC1] I-25 | (1aS,5S,8S,10R,20E,23aS)- 14-chloro-5- cyclopentyl-N- {(1R,2S)-1- [(cyclopropylsulfonyl) carbamoyl]-2- ethenylcyclopropyl}- 1a-methyl-3,6,17- trioxo-1,1a,3,4,5,6,9, 10,19,22,23,23a- dodecahydro-8H,17H- 7,10-methanocyclo propa[18,19][1,10,3,6, 12]dioxatriazacyclo nonadecino[11,12- b]quinazoline-8- carboxamide | 811 | A1, C2, D5 | Exam- ple 5 |
| 26 | 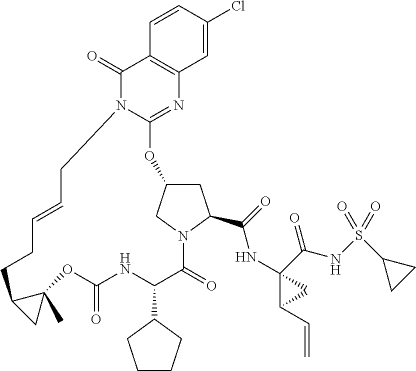 [SMILES: C=C[C@@H]1C[C@]1(NC(=O)[C@@H]1C[C@@H]2CN1C(=O)[C@H](C1CCCC1)NC(=O)O[C@]1(C)C[C@H]1CC/C=C/Cn1c(nc3cc(Cl)ccc3c1=O)O2)C(=O)NS(=O)(=O)C1CC1] I-26 | (1aR,5S,8S,10R,20E,23aR)- 14-chloro-5- cyclopentyl-N- {(1R,2S)-1- [(cyclopropylsulfonyl) carbamoyl]-2- ethenylcyclopropyl}- 1a-methyl-3,6,17- trioxo-1,1a,3,4,5,6,9, 10,19,22,23,23a- dodecahydro-8H,17H- 7,10-methano cyclopropa[18,19][1,10, 3,6,12]dioxatriaza cyclononadecino[11,12- b]quinazoline-8- carboxamide | 811 | A1, C2, D5 | Exam- ple 5 |
| 27 | 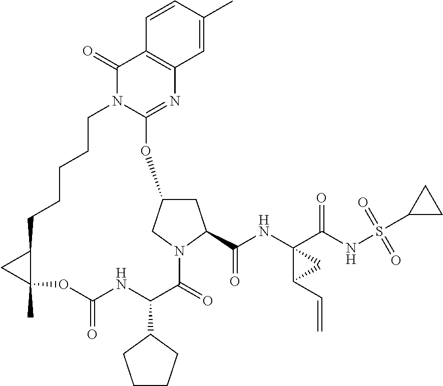 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCn1c(nc3cc(C)ccc3c1=O)O2)C(=O)NS(=O)(=O)C1CC1] I-27 | (1aR,5S,8S,10R,23aR)- 5-cyclopentyl-N- {(1R,2S)-1- [(cyclopropylsulfonyl) carbamoyl]-2- ethenylcyclopropyl}- 1a,14-dimethyl-3,6,17- trioxo-1,1a,3,4,5,6,9, 10,19,22,21,22,23,23a- tetradecahydro- 8H,17H-7,10- methanocyclopropa[18, 19][1,10,3,6,12]dioxatri azacyclononadecino [11,12-b]quinazoline-8- carboxamide | 793 | A1, C2, D6 | Exam- ple 1 |
| 28 | 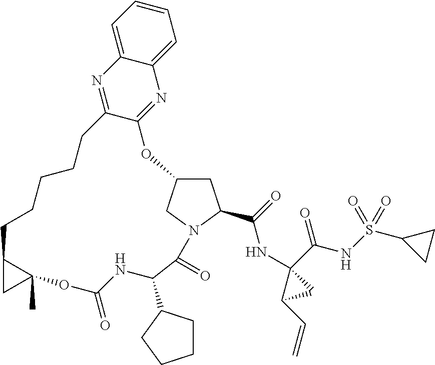 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCc1nc3ccccc3nc1O2)C(=O)NS(=O)(=O)C1CC1] I-28 | (1aR,5S,8S,10R,22aR)- 5-cyclopentyl-N- {(1R,2S)-1- [(cyclopropylsulfonyl) carbamoyl]-2- ethenylcyclopropyl}- 1a-methyl-3,6-dioxo- 1,1a,3,4,5,6,9,10,18,19, 20,21,22,22a- tetradecahydro-8H- 7,10-methanocyclo propa[18,19][1,10,3,6] dioxadiazacyclonona decino[11,12- b]quinoxaline-8- carboxamide | 763 | A1, C3-1, D2 | Exam- ple 1 |
| 29 | 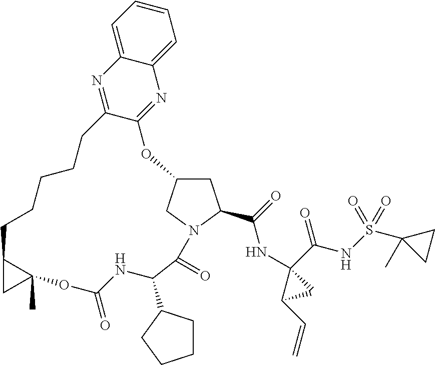 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCc1nc3ccccc3nc1O2)C(=O)NS(=O)(=O)C1(C)CC1] I-29 | (1aR,5S,8S,10R,22aR)- 5-cyclopentyl-N- [(1R,2S)-2-ethenyl-1- {[(1-methylcyclo propyl)sulfonyl] carbamoyl}cyclopropyl]- 1a-methyl-3,6-dioxo- 1,1a,3,4,5,6,9,10,18,19, 20,21,22,22a- tetradecahydro-8H- 7,10-methanocyclo propa[18,19][1,10,3,6] dioxadiazacyclonona decino[11,12- b]quinoxaline-8- carboxamide | 777 | A4, C3-1, D2 | Exam- ple 1 |
| 30 | 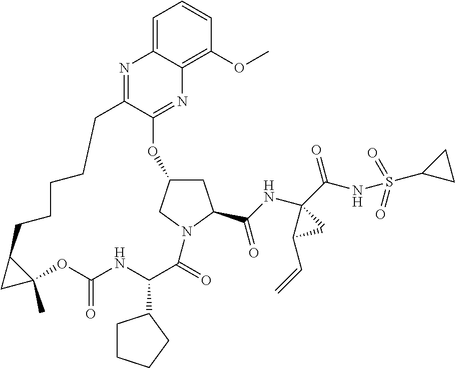 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCc1nc3cccc(OC)c3nc1O2)C(=O)NS(=O)(=O)C1CC1] I-30 | (1aR,5S,8S,10R,22aR)- 5-cyclopentyl-N- {(1R,2S)-1- [(cyclopropylsulfonyl) carbamoyl]-2- ethenylcyclopropyl}- 13-methoxy-1a-methyl- 3,6-dioxo-1,1a,3,4,5, 6,9,10,18,19,20,21,22,22a- tetradecahydro-8H- 7,10-methanocyclo propa[18,19][1,10,3,6] dioxadiazacyclonona decino[11,12- b]quinoxaline-8- carboxamide | 793 | A1, C1, D3 | Exam- ple 1 |
| 31 | 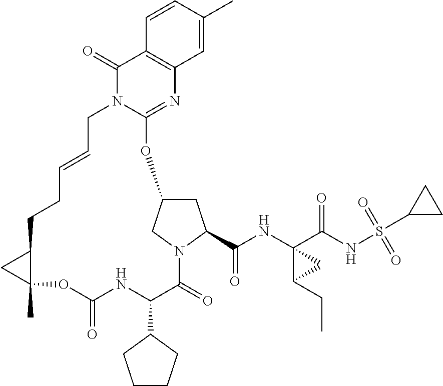 [SMILES: CC[C@@H]1C[C@]1(NC(=O)[C@@H]1C[C@@H]2CN1C(=O)[C@H](C1CCCC1)NC(=O)O[C@]1(C)C[C@H]1CC/C=C/Cn1c(nc3cc(C)ccc3c1=O)O2)C(=O)NS(=O)(=O)C1CC1] I-31 | (1aR,5S,8S,10R,20E,23aR)- 5-cyclopentyl-N- {(1R,2R)-1- [(cyclopropylsulfonyl) carbamoyl]-2- ethylcyclopropyl}- 1a,14-dimethyl-3,6,17- trioxo-1,1a,3,4,5,6, 9,10,19,22,23,23a- dodecahydro-8H,17H- 7,10-methanocyclo propa[18,19][1,10,3,6, 12]dioxatriazacyclo nonadecino[11,12- b]quinazoline-8- carboxamide | 793 | A3, C2, D6 | Exam- ple 5 |
| 32 | 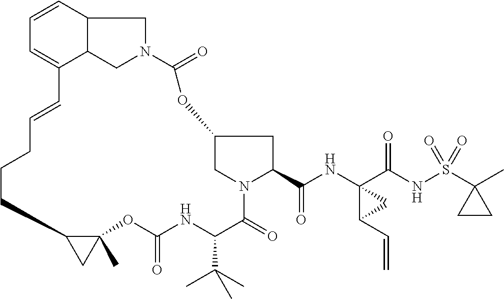 [SMILES: C=C[C@@H]1C[C@]1(NC(=O)[C@@H]1C[C@@H]2CN1C(=O)[C@H](C(C)(C)C)NC(=O)O[C@]1(C)C[C@H]1CCC/C=C/C1=CC=CC3CN(CC13)C(=O)O2)C(=O)NS(=O)(=O)C1(C)CC1] I-32 | (1aR,5S,8S,10R,19E,23aR)- 5-tert-butyl-N- [(1R,2S)-2-ethenyl-1- {[(1-methylcyclo propyl)sulfonyl] carbamoyl}cyclopropyl]- 1a-methyl-3,6,12- trioxo-1a,3,4,5,6,9, 10,14,21,22,23,23a- dodecahydro-1H,8H- 7,10:13,15- dimethanocyclopropa [n][4,13,2,8,11]benzo dioxatriazacyclo docosine-8(12H)- carboxamide | 780 | A4, C4-1, D4 | Exam- ple 5 |

### Example 33

### Comparison of Different Compounds

The compounds of Examples 1 through 32 were compared to the compound of Example 97 of International Patent Application Publication No. WO 2008/057209. The results are shown in Table 2 below. As illustrated in the table, the compounds of formula I, as illustrated by the compounds of Examples 1 through 32, appear to have several advantageous properties, such as improved activity against a one or more mutant compared to the compound of Example 97 of WO 2008/057209.

The activity table provided below illustrates the observed activity:

TABLE 2
NS3/4A Inhibitory Activity (Ki) & Compound Inhibitory Potency (Replicon (nM))

| | Enzyme Activity (nM) | | | gt 1b Replicon EC50 (nM) | |
|---|---|---|---|---|---|
| Compound | gt 1b Ki | gt lb A156V Ki | gt 3a Ki | 10% FBS | 50% NHS |
| WO 2008/057209, | 0.02 | 8.2 | 2.7 | 3.3 | 20 |
| Example 97 | | | | | |
| I-1 | 0.04 | 1.0 | 1.6 | 2 | 7.6 |
| I-2 | — | — | — | — | — |
| I-3 | 0.03 | 3.3 | 0.5 | 6 | 23 |
| I-4 | 0.04 | 0.9 | 0.7 | 7 | 17 |
| I-5 | 0.02 | 1.3 | 1.0 | 3 | 22 |
| I-6 | 0.02 | 0.3 | 2.9 | 3 | 54 |
| I-7 | 0.02 | 2.2 | 8.7 | 4 | 27 |
| I-8 | 0.02 | 11.5 | 0.5 | 2 | 6 |
| I-9 | 0.06 | — | 12.5 | — | — |
| I-10 | 0.02 | 4.2 | 0.2 | 4 | 10 |
| I-11 | 0.03 | — | 0.5 | 4 | 7 |
| I-12 | 0.02 | 1.4 | 12.0 | 5 | 19 |
| I-13 | 0.02 | 0.4 | 32.9 | 4 | 16 |
| I-14 | 0.08 | 6.3 | 112 | — | — |
| I-15 | 0.09 | 8.9 | 93.5 | — | — |
| I-16 | 0.02 | 2.4 | 1.4 | 9 | 27 |
| I-17 | 0.07 | 9.6 | 27.9 | — | — |
| I-18 | 0.07 | 42.5 | 9.2 | 30 | 35 |
| I-19 | 0.5 | 30.0 | 21.8 | — | — |
| I-20 | 0.02 | 27.2 | 58.3 | — | — |
| I-21 | 1.4 | 8.1 | 300 | — | — |
| I-22 | 0.3 | 134.9 | 82.2 | — | — |
| I-23 | 0.02 | 1.2 | 1.0 | 2 | 6 |
| I-24 | 0.02 | 0.9 | 0.7 | 2 | 8.5 |
| I-25 | 0.2 | 142.3 | 41.0 | — | — |
| I-26 | 0.02 | 1.6 | 0.8 | 3 | 13 |
| I-27 | 0.04 | 4.1 | 2.8 | 2 | 6 |
| I-28 | 0.04 | 2.7 | 3.2 | 2 | 10 |
| I-29 | 0.04 | 1.7 | 3.1 | 3 | 9 |
| I-30 | 0.02 | 1.9 | 2.2 | 1 | 5 |
| I-31 | 0.1 | 13.3 | 5.3 | 4 | 10 |
| I-32 | 0.03 | 1.0 | 1.8 | 1 | 11 |

Ki: Inhibition constant; gt: genotype; EC50: Effective concentration achieving 50% viral replicaiton suppression; FBS: Fetal Bovine Serum; NHS: Normal Human Serum.

### Methods

### Measuring NS3/4A Inhibitory Activity (Ki)

Compound described herein can be evaluated for different activities such as the ability to inhibit HCV NS3 activity, HCV replicon activity, and HCV replication activity using techniques well-known in the art. (See, for example, Steven S. Carroll et al., Inhibition of Hepatitis C Virus RNA Replication by 2′-Modified Nucleoside Analogs, 278(14) J. BIOLOGICAL CHEMISTRY 11979 (2003))

One such assay is HCV NS3 protease time-resolved fluorescence (TRF) assay as described below and in Mao et al., Anal Biochem. 373:1-8, 2008 and International Patent Application Publication WO 2006/102087. A NS3 protease assay can be performed, for example, in a final volume of 100 μl assay buffer containing 50 mM HEPES, pH 7.5, 150 mM NaCl, 15% glycerol, 0.15% TRITON X-100, 10 mM DTT, and 0.1% PEG 8000. NS3 and NS4A protease is pre-incubated with various concentrations of inhibitors in DMSO for 30 minutes. The reaction is initiated by adding the TRF peptide substrate (final concentration 100 nM). NS3 mediated hydrolysis of the substrate is quenched after 1 hour at room temperature with 100 μl of 500 mM MES, pH 5.5. Product fluorescence is detected using either a VICTOR V2 or FUSION fluorophotometer (Perkin Elmer Life and Analytical Sciences) with excitation at 340 nm and emission at 615 nm with a 400 μs delay. Testing concentrations of different enzyme forms are selected to result in a signal to background ratio (S/B) of 10-30. IC₅₀ values are derived using a standard four-parameter fit to the data. Kᵢ values are derived from IC₅₀ values using the following formula,

IC₅₀=Kᵢ(1+[S]/K_{M}), Eqn (1),

where [S] is the concentration of substrate peptide in the reaction and K_{M} is the Michaelis constant. See P. Gallinari et al., 38 BIOCHEM. 5620-32 (1999); P. Gallinari et al., 72 J. VIROL. 6758-69 (1998); M. Taliani et al., 240 ANAL. BIOCHEM. 60-67 (1996); Mao et al., Analytical Biochemistry 373: 1-8, 2008.
Measuring Compound Inhibitory Potency (Replicon EC50 (nM))

Measurement of inhibition by compounds was performed using the HCV replicon system. Several different replicons encoding different HCV genotypes or mutations were used. In addition, potency measurements were made using different formats of the replicon assay, including different ways of measurements and different plating formats. See Jan M. Vrolijk et al., A replicons-based bioassay for the measurement of interferons in patients with chronic hepatitis C, 110 J. VIROLOGICAL METHODS 201 (2003); Steven S. Carroll et al., Inhibition of Hepatitis C Virus RNA Replication by 2′-Modified Nucleoside Analogs, 278(14) J. BIOLOGICAL CHEMISTRY 11979 (2003). However, the underlying principles are common to all of these determinations, and are outlined below.

Stable neomycin phosphotransferase encoding replicon-harboring cell lines were used, so all cell lines were maintained under G418 selection prior to the assay. In some cases the cell lines encoded a luciferase:Neor fusion and could be assayed either directly by determination of RNA copy number, or indirectly through measurement of the luciferase activity.

To initiate an assay, replicon cells were plated in the presence of a dilution series of test compound in the absence of G418. Typically, the assays were performed in a 96-well plate format for manual operation, or a 384 well plate in an automated assay. Replicon cells and compound were incubated for 24-48 hours, depending on the assay. At the end of the assay, cells are washed free of media and compound and then lysed. For direct quantitation, RNA levels were measured by ³²P-probe hybridization and protection, or in a TAQMAN based assay and normalized to cellular cyclophilin A RNA levels. In some cases, luciferase activity was measured using a conventional luciferase assay. In all cases EC50 determinations were calculated as a percent of a DMSO control by fitting the data to a four parameter fit function.

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A compound of formula I: 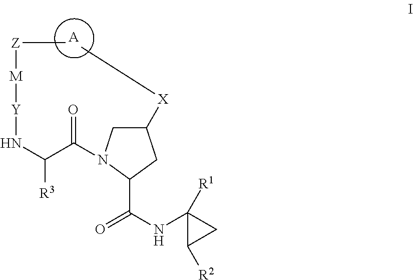
or a pharmaceutically acceptable salt or hydrate thereof, wherein: 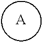
is one or more rings selected from the group consisting of:
a) aryl rings,
b) C₃-C₈ cycloalkyl rings, and
c) heterocyclic rings in which the heterocyclic ring system attaches to Z and X at points that are two independently selected ring atoms that are either two carbon ring atoms or one carbon ring atom and one nitrogen ring atom, and the heterocyclic ring system is selected from the group consisting of:
i) 5- or 6-membered saturated or unsaturated monocyclic rings with 1, 2, or 3 heteroatom ring atoms independently selected from the group consisting of N, O or S,
ii) 8-, 9- or 10-membered saturated or unsaturated bicyclic rings with 1, 2, or 3 heteroatom ring atoms independently selected from the group consisting of N, O or S, and
iii) 11- to 15-membered saturated or unsaturated tricyclic rings with 1, 2, 3, or 4 heteroatom ring atoms independently selected from the group consisting of N, O or S,
wherein 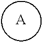
is substituted with 0 to 4 independently selected substituents R⁴, R⁵ or oxo; wherein for stable heterocyclic rings containing S or N, the heterocyclic ring is unsubstituted at the S or N atom or is substituted at the S by oxo; wherein the R⁴ and R⁵ substitutions are located on one or more ring atoms selected from C and N;
X is selected from the group consisting of —O—, —CH₂O—, —NHC(O)O—, —CH₂NHC(O)O—, —C≡CCH₂O—, —C(O)O—, —(CH₂)₃O—, —OC(O)NH—, —(CH₂)₂C(O)NH—, —C(O)NH— and a direct bond;
R¹ is selected from the group consisting of —CO₂R⁶, —CONR⁶SO₂R⁷, —CONR⁶SO₂NR⁸R⁹, tetrazolyl, —CONHP(O)R¹⁰R¹¹, and —P(O)R¹⁰R¹¹;
R² is selected from the group consisting of C₁-C₆ alkyl, C₂-C₆ alkenyl, and C₃-C₈ cycloalkyl, wherein the R² are substituted with 0 to 3 independently selected halogen atoms;
R³ is selected from the group consisting of H, C₁-C₈ alkyl, C₃-C₈ cycloalkyl, C₃-C₈ cycloalkyl(C₁-C₈)alkyl, phenyl(C₁-C₈)alkyl, naphthyl(C₁-C₈)alkyl, and Het groups, wherein when R³ is not H, the R³ is substituted with 0 to 3 substituents independently selected from the group consisting of halogen atoms, —OR⁶, —SR⁶, —N(R⁶)₂, —N(C₁-C₆ alkyl)O(C₁-C₆ alkyl), C₁-C₆ alkyl, C₁-C₆ haloalkyl, halo(C₁-C₆ alkoxy), —NO₂, —CN, —CF₃, —SO₂(C₁-C₆ alkyl), —S(O)(C₁-C₆ alkyl), —NR⁶SO₂R⁷, SO₂N(R⁷)₂, —NHCOOR⁷, —NHCOR⁷, —NHCONHR⁷, —CO₂R⁶, —C(O)R⁶, and —CON(R⁶)₂;
Het is selected from the group consisting of substituted and unsubstituted 5- and 6-membered saturated heterocyclic rings having 1 or 2 heteroatoms independently selected from N, O and S;
Y is selected from the group consisting of —C(O)—, —SO₂—, —OC(O)—, —C(O)N(R¹²)L- and -LN(R¹²)C(O)—, where
R¹² is selected from the group consisting of H, C₁-C₆ alkyl and C₁-C₆ alkenyl groups,
L is selected from the group consisting of a direct bond, -G-(C₁-C₆ alkylene)-, —(C₁-C₆ alkylene)-G-, -G-(C₁-C₆ alkenylene)-, and —(C₁-C₆ alkenylene)-G-, groups, where the G is selected from the group consisting of a direct bond, —O—, —N— and —S—, the alkylene and alkenylene groups are substituted with 0 to 4 substituents R¹³ independently selected from the group consisting of C₁-C₆ alkyl and C₁-C₆ alkenyl groups, and
the R¹² and R¹³ may be taken together to form a 3- to 6-membered ring containing 0 to 3 heteroatoms selected from N, O and S;
M is selected from the group consisting of C₁-C₁₂ alkylenes, C₂-C₁₂ alkenylenes and C₂-C₁₂ alkynylenes, wherein:
the M contains 0 or 1-O— moiety in place of a methylene moiety, and
the M is substituted with from 0 to 4 substituents R¹⁴, wherein:
each R¹⁴ is independently selected from the group consisting of C₁-C₈ alkyl, ═CH₂, C₃-C₈ cycloalkyl(C₁-C₈ alkyl), and aryl(C₁-C₈ alkyl), and
any substituent R¹⁴ may be taken together with any adjacent substituent R¹⁴ or any adjacent substituent R¹² or R¹³ to form a 3- to 6-membered ring containing 0 to 3 heteroatoms independently selected from the group consisting of N, O and S;
Z is selected from the group consisting of —C(O)— and a direct bond;
R⁴ is selected from the group consisting of H, halogen atoms, —OH, C₁-C₆ alkoxy, C₁-C₆ alkyl, —CN, —CF₃, —OCF₃, —C(O)OH, —C(O)CH₃, —SR⁶, —SO₂(C₁-C₆ alkyl), C₃-C₈ cycloalkyl, C₃-C₈ cycloalkoxy, C₁-C₆ haloalkyl, —N(R¹⁵)₂, phenyl, naphthyl, —O-phenyl, —O-naphthyl, heteroaryl and heterocyclyl groups; wherein:
the R⁴ heteroaryl is selected from the group consisting of 5- and 6-membered aromatic rings having 1, 2 or 3 heteroatoms independently selected from N, O and S, and the R⁴ heteroaryl is attached through a ring atom selected from C or N,
the R⁴ heterocyclyl is selected from the group consisting of 5- to 7-membered saturated or unsaturated non-aromatic rings having 1, 2, 3 or 4 heteroatoms independently selected from N, O and S, and the R⁴ heterocyclyl is attached through a ring atom selected from C or N, and
the R⁴ heteroaryl, heterocyclyl, cycloalkyl, cycloalkoxy, alkyl and alkoxy groups are substituted with 0 to 4 substituents independently selected from the group consisting of halogen atoms, —OR⁶, —SR⁶, —N(R⁶)₂, —N(C₁-C₆ alkyl)O(C₁-C₆ alkyl), C₁-C₆ alkyl, C₁-C₆ haloalkyl, halo(C₁-C₆ alkoxy), —NO₂, —CN, —CF₃,—SO₂(C₁-C₆ alkyl), —S(O)(C₁-C₆ alkyl), —NR⁶SO₂R⁷, SO₂N(R⁷)₂, —NHCOOR⁷, —NHCOR⁷, —NHCONHR⁷, —CO₂R⁶, —C(O)R⁶, and —CON(R⁶)₂, and 2 adjacent substituents of the R⁴ heteroaryl, heterocyclyl, cycloalkyl, cycloalkoxy, alkyl and alkoxy groups may be taken together to form a 3- to 6-membered cyclic ring containing 0 to 3 heteroatoms independently selected from N, O and S;
each R⁵ is independently selected from the group consisting of H, halogen atoms, —OR⁶, C₁-C₆ alkyl, —CN, —CF₃, —NO₂, —SR⁶, —CO₂R⁶, —CON(R⁶)₂, —C(O)R⁶, —N(R⁶)C(O)R⁶, —SO₂(C₁-C₆ alkyl), —S(O)(C₁-C₆ alkyl), C₃-C₈ cycloalkyl, C₃-C₈ cycloalkoxy, C₁-C₆ haloalkyl, —N(R⁶)₂, —N(C₁-C₆ alkyl)O(C₁-C₆ alkyl), halo(C₁-C₆ alkoxy), —NR⁶SO₂R⁶, —SO₂N(R⁶)₂, —NHCOOR⁶, —NHCONHR⁶, phenyl, naphthyl, heteroaryl and heterocyclyl groups, wherein
the R⁵ heteroaryl is selected from the group consisting of 5- and 6-membered aromatic rings having 1, 2 or 3 heteroatoms independently selected from N, O and S, and the R⁵ heteroaryl is attached through a ring atom selected from C or N,
the R⁵ heterocyclyl is selected from the group consisting of 5- to 7-membered saturated or unsaturated non-aromatic rings having 1, 2, 3 or 4 heteroatoms independently selected from N, O and S, and the R⁵ heterocyclyl is attached through a ring atom selected from C or N;
each R⁶ is independently selected from the group consisting of H, C₁-C₆ alkyl and benzyl, wherein each R⁶ is independently substituted with 0 to 4 substituents selected from the group consisting of halogen atoms, C₁-C₆ alkyl, C₁-C₆ haloalkyl, phenyl, naphthyl, C₃-C₈ cycloalkyl, heteroaryl, heterocyclyl, halo(C₁-C₆ alkoxy), —OH, —O(C₁-C₆ alkyl), —SH, —S(C₁-C₆ alkyl), —NH₂, —NH(C₁-C₆ alkyl), —N(C₁-C₆ alkyl)₂, —C(O)(C₁-C₆ alkyl), NO₂, —CN, —CF₃, —SO₂(C₁-C₆ alkyl), —S(O)(C₁-C₆ alkyl), —N(C₁-C₆ alkyl)SO₂(C₁-C₆ alkyl), —SO₂N(C₁-C₆ alkyl)₂, —NHCOO(C₁-C₆ alkyl), —NHCO(C₁-C₆ alkyl), —NHC ONH(C₁-C₆ alkyl), —CO₂(C₁-C₆ alkyl), and —C(O)N(C₁-C₆ alkyl)₂;
R⁷ is selected from the group consisting of H, C₁-C₆ alkyl, C₃-C₆ cycloalkyl, C₃-C₆ cycloalkyl(C₁-C₅)alkyl, phenyl, naphthyl, phenyl(C₁-C₄)alkyl, naphthyl(C₁-C₄)alkyl, heteroaryl, heteroaryl(C₁-C₄ alkyl), heterocyclyl, and heterocyclyl(C₁-C₈ alkyl) groups, wherein
the R⁷ are substituted with 0 to 2 independently selected R⁵ substituents,
each R⁷ heteroaryl is independently selected from the group consisting of 5- and 6-membered aromatic rings having 1, 2 or 3 heteroatoms independently selected from N, O and S, and the R⁷ heteroaryl is attached through a ring atom selected from C or N, and
each R⁷ heterocyclyl is independently selected from the group consisting of 5- to 7-membered saturated or unsaturated non-aromatic rings having 1, 2, 3 or 4 heteroatoms independently selected from N, O and S, and the R⁷ heterocyclyl is attached through a ring atom selected from C or N;
R⁸ is selected from the group consisting of H, C₁-C₈ alkyl, C₃-C₈ cycloalkyl, C₃-C₈ cycloalkyl(C₁-C₈ alkyl), phenyl, naphthyl, phenyl(C₁-C₄)alkyl, naphthyl(C₁-C₄)alkyl, heteroaryl, heterocyclic, heteroaryl(C₁-C₄ alkyl), and heterocyclyl(C₁-C₈ alkyl) groups, wherein
the R⁸ are substituted with 0 to 4 substituents selected from the group consisting of phenyl, naphthyl, C₃-C₈ cycloalkyl, heteroaryl, heterocyclyl, C₁-C₆ alkyl, halo(C₁-C₆ alkoxy), halogen atoms, —OR⁶, —SR⁶, —N(R⁶)₂, —N(C₁-C₆ alkyl)O(C₁-C₆ alkyl), C₁-C₆ alkyl, —C(O)R⁶, C₁-C₆ haloalkyl, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂,
each R⁸ heteroaryl is independently selected from the group consisting of 5- and 6-membered aromatic rings having 1, 2 or 3 heteroatoms independently selected from N, O and S, and the R⁸ heteroaryl is attached through a ring atom selected from C or N,
each R⁸ heterocyclyl is independently selected from the group consisting of 5- to 7-membered saturated or unsaturated non-aromatic rings having 1, 2, 3 or 4 heteroatoms independently selected from N, O and S, and the R⁸ heterocyclyl is attached through a ring atom selected from C or N, and
2 adjacent substituents of the R⁸ may be taken together to form a 3- to 6-membered ring containing 0 to 3 heteroatoms independently selected from the group consisting of N, O and S;
R⁹ is selected from the group consisting of H, C₁-C₈ alkyl, C₃-C₈ cycloalkyl, C₃-C₈ cycloalkyl(C₁-C₈ alkyl), C₁-C₈ alkoxy, C₃-C₈ cycloalkoxy, phenyl, naphthyl, phenyl(C₁-C₄)alkyl, naphthyl(C₁-C₄)alkyl, heteroaryl, heterocyclyl, heteroaryl(C₁-C₄ alkyl), or heterocyclyl(C₁-C₈ alkyl) groups, wherein
the R⁹ are substituted with 0 to 4 substituents selected from the group consisting of phenyl, naphthyl, C₃-C₈ cycloalkyl, heteroaryl, heterocyclyl, C₁-C₆ alkyl, halo(C₁-C₆ alkoxy), halogen atoms, —OR⁶, —SR⁶, —N(R⁶)₂, —N(C₁-C₆ alkyl)O(C₁-C₆ alkyl), C₁-C₆ alkyl, —C(O)R⁶, C₁-C₆ haloalkyl, —NO₂, —CN, —CF₃₅—SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂,
each R⁹ heteroaryl is independently selected from the group consisting of 5- and 6-membered aromatic rings having 1, 2 or 3 heteroatoms independently selected from N, O and S, and the R⁹ heteroaryl is attached through a ring atom selected from C or N,
each R⁹ heterocyclyl is independently selected from the group consisting of 5- to 7-membered saturated or unsaturated non-aromatic rings having 1, 2, 3 or 4 heteroatoms independently selected from N, O and S, and the R⁹ heterocyclyl is attached through a ring atom selected from C or N, and
2 adjacent substituents of the R⁹ may be taken together to form a 3- to 6-membered ring containing 0 to 3 heteroatoms independently selected from the group consisting of N, O and S, and
R⁸ and R⁹ may be taken together, with the N to which they are attached, to form a 4- to 8-membered monocyclic ring containing 0 to 2 additional heteroatoms independently selected from N, O and S;
each R¹⁰ is independently selected from the group consisting of H, C₁-C₆ alkyl, C₁-C₄ alkenyl, —OR¹⁶, —N(R⁶)—V—CO₂R⁶, —O—V—CO₂R⁶, —S—V—CO₂R⁶, —N(R⁶)(R¹⁶), —R¹⁷, and —N(R⁶)SO₂R⁷;
each R¹¹ is independently selected from the group consisting of H, —OR¹⁶, —N(R⁶)—V—CO₂R⁶, —O—V—CO₂R⁶, —S—V—CO₂R⁶, and —N(R⁶)(R¹⁶);
R¹⁰ and R¹¹ may be taken together, with the phosphorus atom to which they are attached, to form a 5- to 7-membered monocyclic ring;
each V is independently selected from the group consisting of —CH(R¹⁸)— and —(C₁-C₄ alkylene)-CH(R¹⁸)—;
each R¹⁵ is independently selected from the group consisting of H, C₁-C₆ alkyl, C₃-C₆ cycloalkyl, C₃-C₆ cycloalkyl(C₁-C₅)alkyl, phenyl, naphthyl, phenyl(C₁-C₄)alkyl, naphthyl(C₁-C₄)alkyl, heteroaryl, heteroaryl(C₁-C₄ alkyl), heterocyclyl, and heterocyclyl(C₁-C₈ alkyl) groups, wherein
when R¹⁵ is not H, the R¹⁵ are substituted with 0 to 2 R⁵ substituents,
each R¹⁵ heteroaryl is independently selected from the group consisting of 5- and 6-membered aromatic rings having 1, 2 or 3 heteroatoms independently selected from N, O and S, and the R¹⁵ heteroaryl is attached through a ring atom selected from C or N, and
each R¹⁵ heterocyclyl is independently selected from the group consisting of 5- to 7-membered saturated or unsaturated non-aromatic rings having 1, 2, 3 or 4 heteroatoms independently selected from N, O and S, and the R¹⁵ heterocyclyl is attached through a ring atom selected from C or N, and
the R¹⁵ may be taken together with the atom to which it is attached and a second R¹⁵ substituent to form a 4- to 7-membered heterocyclic ring;
each R¹⁶ is independently selected from the group consisting of H, C₁-C₆ alkyl, C₂-C₆ alkenyl, C₃-C₈ cycloalkyl, aryl, heteroaryl, and heterocyclyl groups, wherein
when R¹⁶ is not H, the R¹⁶ is substituted with 0 to 2 substituents independently selected from the group consisting of phenyl, naphthyl, phenyl(C₁-C₄ alkyl), naphthyl(C₁-C₄ alkyl), C₃-C₈ cycloalkyl, C₃-C₈ cycloalkyl(C₁-C₄ alkyl), heteroaryl, heteroaryl(C₁-C₄ alkyl), heterocyclyl, heterocyclyl(C₁-C₄ alkyl), C₁-C₆ alkyl, halogen atoms, —OC(O)OR⁷, —OC(O)R⁷, —OR⁶, —SR⁶, —N(R⁶)₂, —C(O)R⁶, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂,
each R¹⁶ heteroaryl is independently selected from the group consisting of 5- and 6-membered aromatic rings having 1, 2 or 3 heteroatoms independently selected from N, O and S, and the R¹⁶ heteroaryl is attached through a ring atom selected from C or N,
each R¹⁶ heterocyclyl is independently selected from the group consisting of 5- to 7-membered saturated or unsaturated non-aromatic rings having 1, 2, 3 or 4 heteroatoms independently selected from N, O and S, and the R¹⁶ heterocyclyl is attached through a ring atom selected from C or N, and
2 adjacent substituents of the R¹⁶ may be taken together to form a 3- to 6-membered ring containing 0 to 3 heteroatoms independently selected from the group consisting of N, O and S;
R¹⁷ is selected from the group consisting of H, C₁-C₆ alkyl, C₂-C₆ alkenyl, phenyl, naphthyl and heteroaryl, wherein
each R¹⁷ heteroaryl is independently selected from the group consisting of 5- and 6-membered aromatic rings having 1, 2 or 3 heteroatoms independently selected from N, O and S, and the R¹⁷ heteroaryl is attached through a ring atom selected from C or N, and
the R¹⁷ phenyl, naphthyl or heteroaryl may be substituted with 0 to 2 substituents independently selected from the group consisting of C₁-C₆ alkyl, halogen atoms, —OC(O)OR⁷, —OC(O)R⁷, —OR⁶, SR⁶, —N(R⁶)₂, —C(O)R⁶, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂; and
each R¹⁸ is independently selected from the group consisting of H, C₁-C₆ alkyl, C₂-C₆ alkenyl, C₃-C₈ cycloalkyl, phenyl, naphthyl, heteroaryl, and heterocyclyl groups, wherein
the R¹⁸ are substituted with 0 to 2 substituents independently selected from the group consisting of C₁-C₆ alkyl, halogen atoms, —OC(O)OR⁷, —OC(O)R⁷, —OR⁶, —SR⁶, —N(R⁶)₂, —C(O)R⁶, —NO₂, —CN, —CF₃, —SO₂(C₁-C₆ alkyl), —S(O)(C₁-C₆ alkyl), —NR⁶SO₂R⁷, —SO₂N(R⁷)₂, —NHCOOR⁷, —NHCOR⁷, —NHCONHR⁷, —CO₂R⁶, and —C(O)N(R⁶)₂,
each R¹⁸ heteroaryl is independently selected from the group consisting of 5- and 6-membered aromatic rings having 1, 2 or 3 heteroatoms independently selected from N, O and S, and the R¹⁸ heteroaryl is attached through a ring atom selected from C or N,
each R¹⁸ heterocyclyl is independently selected from the group consisting of 5- to 7-membered saturated or unsaturated non-aromatic rings having 1, 2, 3 or 4 heteroatoms independently selected from N, O and S, and the R¹⁸ heterocyclyl is attached through a ring atom selected from C or N, and
2 adjacent substituents of the R¹⁸ are optionally taken together to form a 3- to 6-membered ring containing 0 to 3 heteroatoms independently selected from the group consisting of N, O and S.

2. The compound according to claim 1, wherein 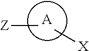
is selected from the group consisting of: 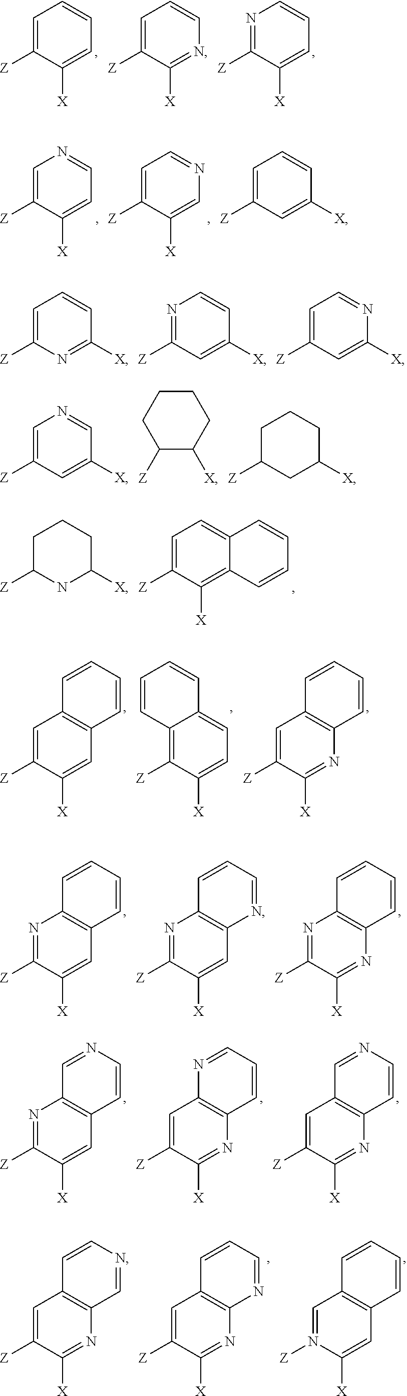 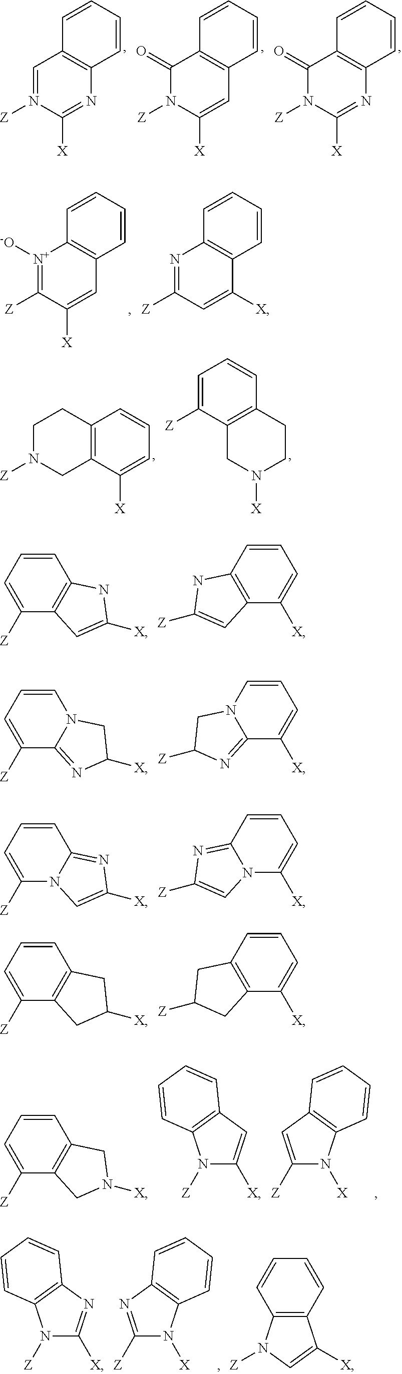 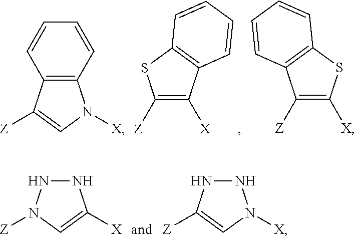
wherein said 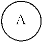
is substituted with 0 to 4 independently selected substituents R⁴, R⁵ or oxo; wherein for stable heterocyclic rings containing S or N, the heterocyclic ring is unsubstituted at the S or N atom or is substituted at the S by oxo; wherein said R⁴ and R⁵ substitutions are located on one or more ring atoms selected from C and N.

3. The compound according to claim 2, wherein 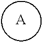
is unsubstituted or mono-substituted with a moiety selected from the group consisting of —Br, —Cl, —CN, phenyl, —O-phenyl, —O-benzyl, —OCF₃, —OCH₃, —OH, C₁-C₆ alkoxy, C₁-C₆ alkyl, —CF₃, —C(O)OH, and —C(O)CH₃.

4. The compound according claim 1, wherein X is selected from the group consisting of —O— and —C(O)O—.

5. The compound according claim 1, wherein R¹ is selected from the group consisting of —CO₂R⁶ and —CONR⁶SO₂R⁷.

6. The compound according to claim 5, wherein R¹ is selected from the group consisting of

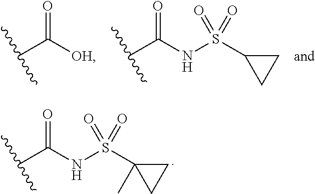

7. The compound according claim 1, wherein R² is selected from the group consisting of C₁-C₆ alkyl and C₂-C₆ alkenyl.

8. The compound according to claim 7, wherein R² is selected from the group consisting of —CH═CH₂, —CH₂CH₃, and —CH₂CH═CH₂.

9. The compound according claim 1, wherein R³ is selected from the group consisting of H, C₁-C₈ alkyl and C₃-C₈ cycloalkyl.

10. The compound according to claim 9, wherein R³ is selected from the group consisting of —C(CH₃)₃, —(CH₂)₃CH₃, cyclohexyl, and —CH(CH₃)₂.

11. The compound according claim 1, wherein Y is selected from the group consisting of —OC(O)—, —C(O)N(D)L- and -LN(D)C(O)—.

12. The compound according claim 1, wherein M is selected from the group consisting of C₁-C₁₂ alkylene or C₂-C₁₂ alkenylene, wherein M is substituted with 0 to 3 substituents R¹⁴ selected from the group consisting of C₁-C₈ alkyl, and ═CH₂.

13. The compound according to claim 12, wherein Z-M-Y is selected from the group consisting of

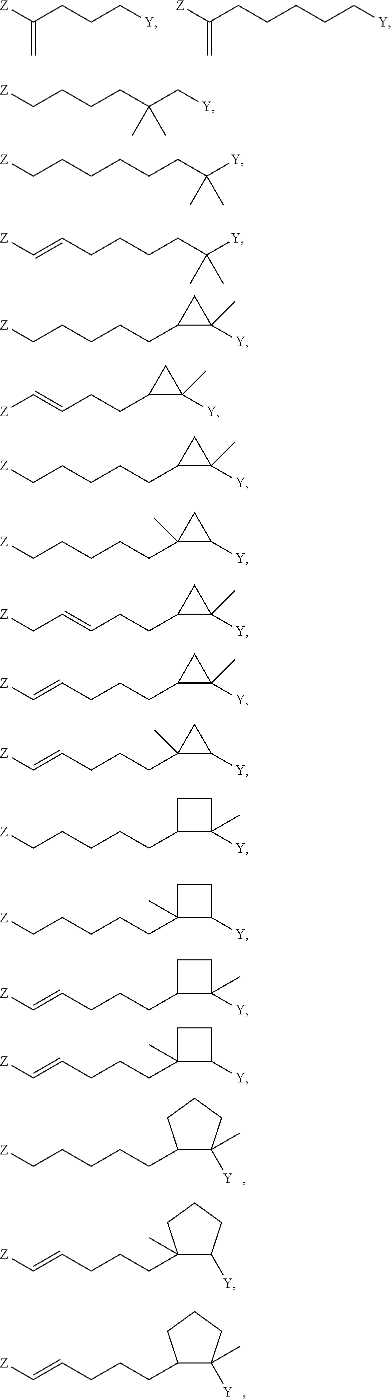

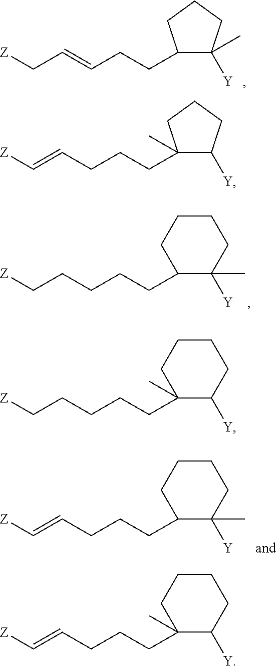

14. (canceled)

15. The compound according claim 1, wherein one or more substituents R¹⁴ are taken together and/or with one or more substituents chosen from substituents R¹² and R¹³ to form a 3- to 6-membered ring containing 0 to 3 heteroatoms selected from the group consisting of N, O and S.

16. A compound of formula I: 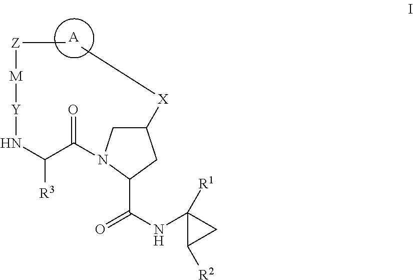
or a pharmaceutically acceptable salt or hydrate thereof, wherein: 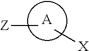
is selected from the group consisting of: 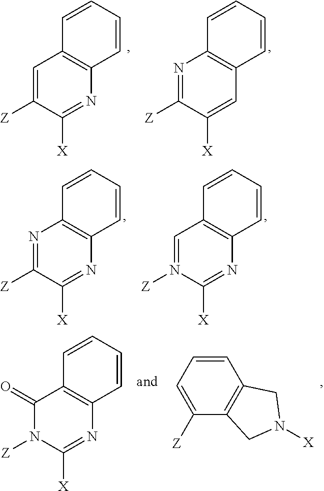
wherein said 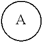
is substituted with 0 to 2 independently selected substituents —Cl, —O-benzyl, —OCH₃, —OH, —CH₃;
X is selected from the group consisting of —O— and —C(O)O—;
R¹ is selected from the group consisting of 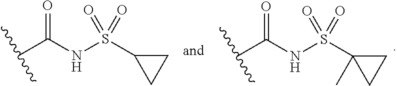 [SMILES: *C(=O)NS(=O)(=O)C1CC1 ; "and" ; *C(=O)NS(=O)(=O)C1(C)CC1 ; "."]
R² is selected from the group consisting of —CH═CH₂ and —CH₂CH₃;
R³ is selected from the group consisting of —C(CH₃)₃ and cyclohexyl;
Y is —OC(O)—;
Z-M-Y is selected from the group consisting of 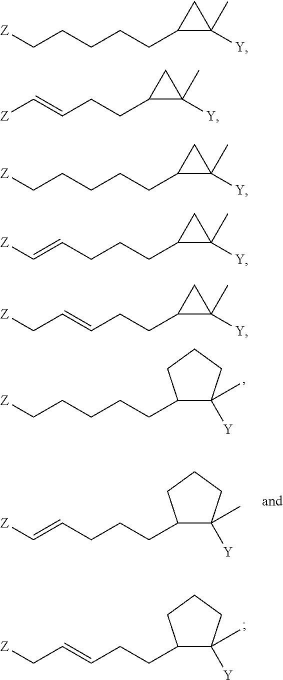
and
Z is a direct bond.

17. A compound selected from the group consisting of:

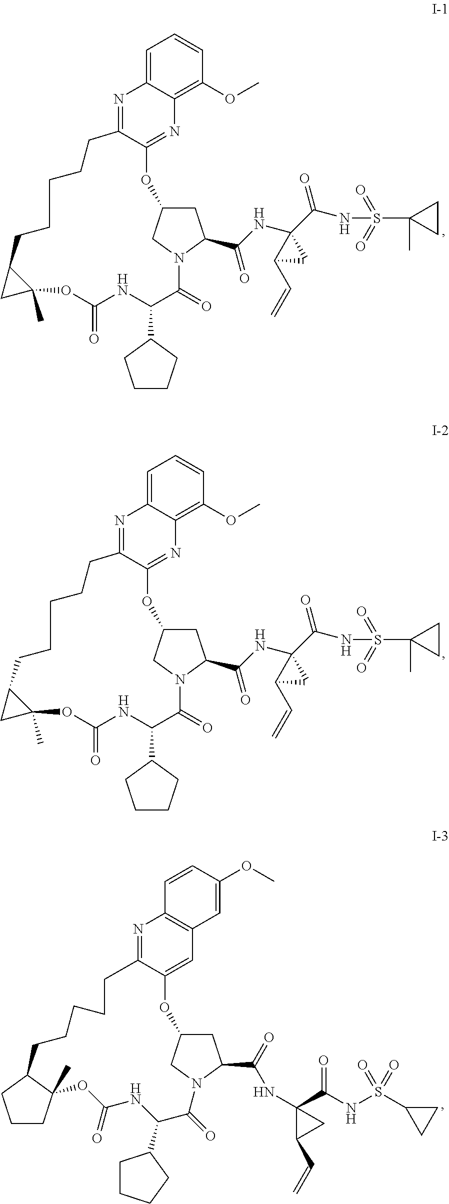 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCc1nc3cccc(OC)c3nc1O2)C(=O)NS(=O)(=O)C1(C)CC1 ; C=C[C@@H]1C[C@]1(NC(=O)[C@@H]1C[C@@H]2CN1C(=O)[C@H](C1CCCC1)NC(=O)O[C@@]1(C)C[C@@H]1CCCCCc1nc3cccc(OC)c3nc1O2)C(=O)NS(=O)(=O)C1(C)CC1 ; C=C[C@@H]1C[C@]1(NC(=O)[C@@H]1C[C@@H]2CN1C(=O)[C@H](C1CCCC1)NC(=O)O[C@]1(C)CCC[C@H]1CCCCCc1nc3ccc(OC)cc3cc1O2)C(=O)NS(=O)(=O)C1CC1]

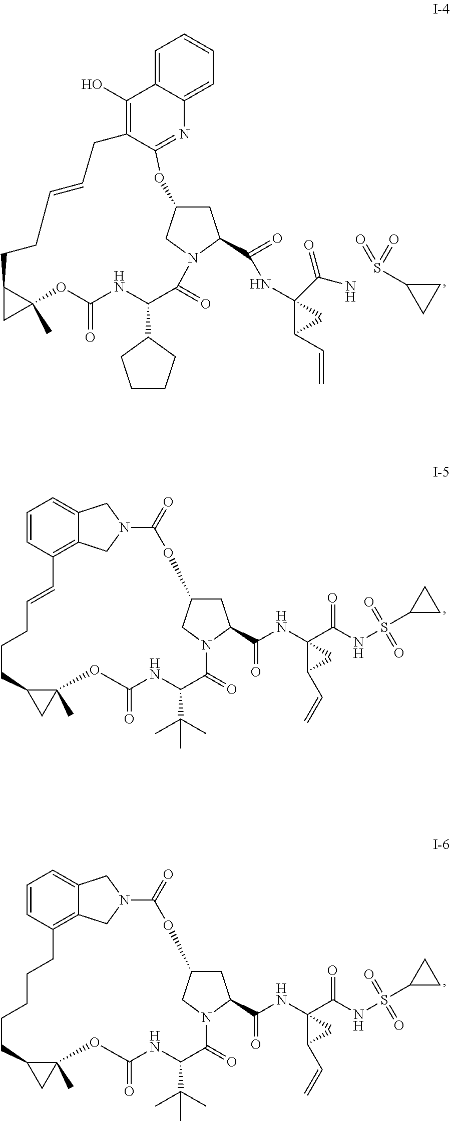

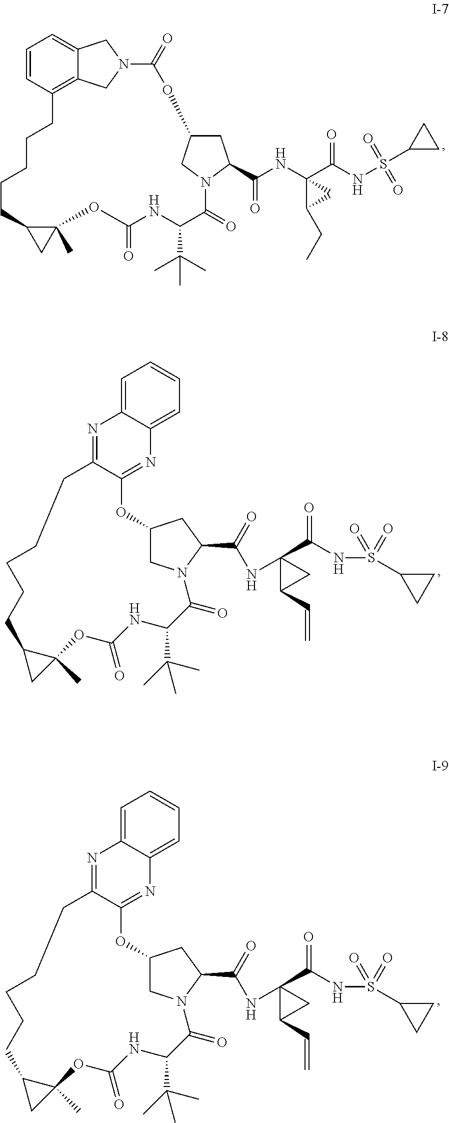 [SMILES: CC[C@@H]1C[C@]1(NC(=O)[C@@H]1C[C@@H]2CN1C(=O)[C@H](C(C)(C)C)NC(=O)O[C@]1(C)C[C@H]1CCCCCc1cccc3c1CN(C3)C(=O)O2)C(=O)NS(=O)(=O)C1CC1 ; C=C[C@@H]1C[C@]1(NC(=O)[C@@H]1C[C@@H]2CN1C(=O)[C@H](C(C)(C)C)NC(=O)O[C@]1(C)C[C@H]1CCCCCc1nc3ccccc3nc1O2)C(=O)NS(=O)(=O)C1CC1 ; C=C[C@@H]1C[C@]1(NC(=O)[C@@H]1C[C@@H]2CN1C(=O)[C@H](C(C)(C)C)NC(=O)O[C@@]1(C)C[C@@H]1CCCCCc1nc3ccccc3nc1O2)C(=O)NS(=O)(=O)C1CC1]

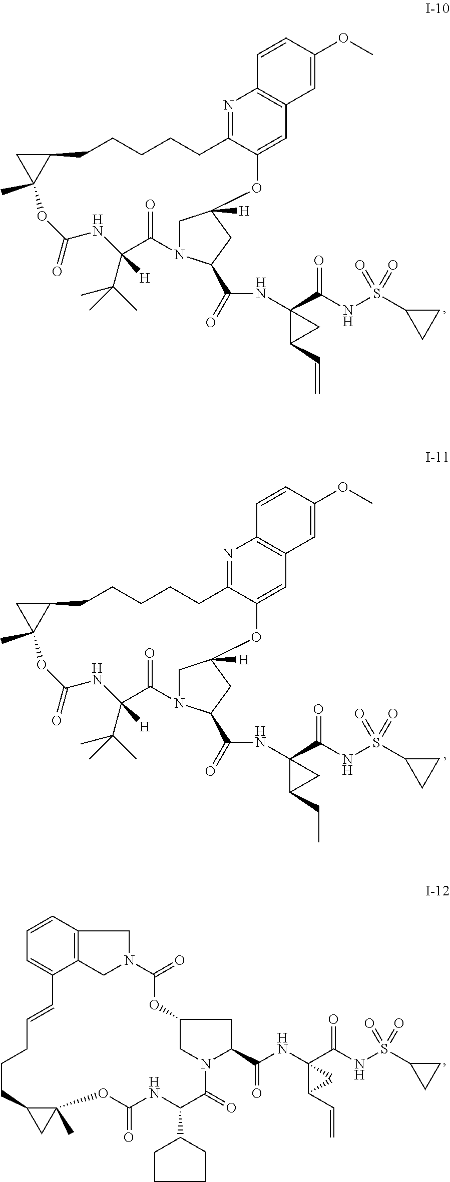 [SMILES: C=C[C@@H]1C[C@]1(NC(=O)[C@@H]1C[C@@H]2CN1C(=O)[C@H](C(C)(C)C)NC(=O)O[C@]1(C)C[C@H]1CCCCCc1nc3ccc(OC)cc3cc1O2)C(=O)NS(=O)(=O)C1CC1 ; CC[C@@H]1C[C@]1(NC(=O)[C@@H]1C[C@@H]2CN1C(=O)[C@H](C(C)(C)C)NC(=O)O[C@]1(C)C[C@H]1CCCCCc1nc3ccc(OC)cc3cc1O2)C(=O)NS(=O)(=O)C1CC1 ; C=C[C@@H]1C[C@]1(NC(=O)[C@@H]1C[C@@H]2CN1C(=O)[C@H](C1CCCC1)NC(=O)O[C@]1(C)C[C@H]1CCC/C=C/c1cccc3c1CN(C3)C(=O)O2)C(=O)NS(=O)(=O)C1CC1]

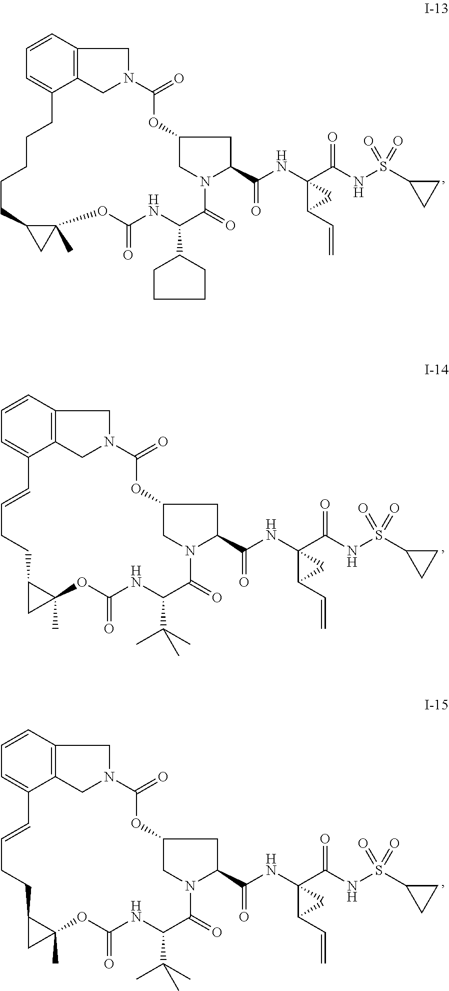 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCc1cccc3c1CN(C3)C(=O)O2)C(=O)NS(=O)(=O)C1CC1 ; C=C[C@@H]1C[C@]1(NC(=O)[C@@H]1C[C@@H]2CN1C(=O)[C@H](C(C)(C)C)NC(=O)O[C@@]1(C)C[C@@H]1CC/C=C/c1cccc3c1CN(C3)C(=O)O2)C(=O)NS(=O)(=O)C1CC1 ; C=C[C@@H]1C[C@]1(NC(=O)[C@@H]1C[C@@H]2CN1C(=O)[C@H](C(C)(C)C)NC(=O)O[C@]1(C)C[C@H]1CC/C=C/c1cccc3c1CN(C3)C(=O)O2)C(=O)NS(=O)(=O)C1CC1]

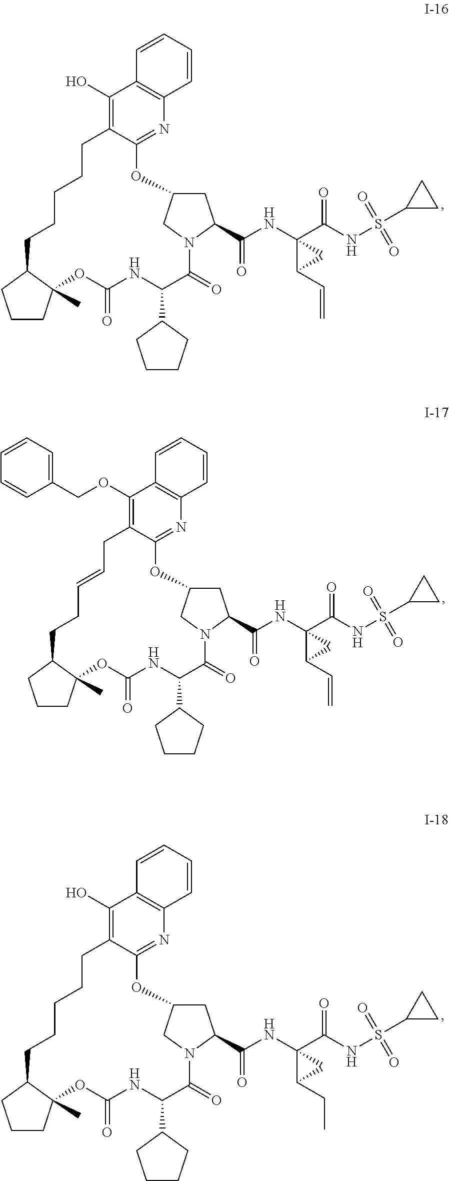 [SMILES: C=C[C@@H]1C[C@]1(NC(=O)[C@@H]1C[C@@H]2CN1C(=O)[C@H](C1CCCC1)NC(=O)O[C@]1(C)CCC[C@H]1CCCCCc1c(nc3ccccc3c1O)O2)C(=O)NS(=O)(=O)C1CC1 ; C=C[C@@H]1C[C@]1(NC(=O)[C@@H]1C[C@@H]2CN1C(=O)[C@H](C1CCCC1)NC(=O)O[C@]1(C)CCC[C@H]1CC/C=C/Cc1c(nc3ccccc3c1OCc1ccccc1)O2)C(=O)NS(=O)(=O)C1CC1 ; CC[C@@H]1C[C@]1(NC(=O)[C@@H]1C[C@@H]2CN1C(=O)[C@H](C1CCCC1)NC(=O)O[C@]1(C)CCC[C@H]1CCCCCc1c(nc3ccccc3c1O)O2)C(=O)NS(=O)(=O)C1CC1]

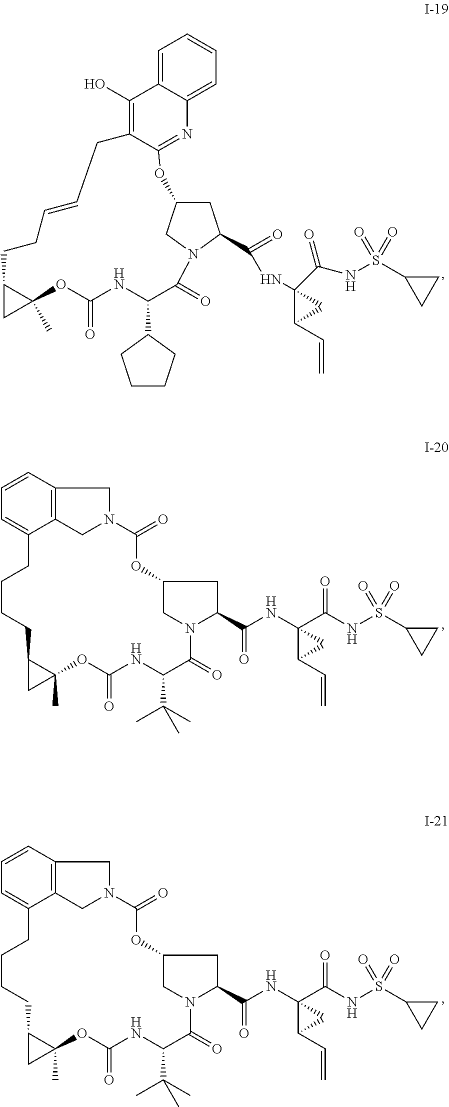 [SMILES: C=C[C@@H]1C[C@]1(NC(=O)[C@@H]1C[C@@H]2CN1C(=O)[C@H](C1CCCC1)NC(=O)O[C@@]1(C)C[C@@H]1CC/C=C/Cc1c(nc3ccccc3c1O)O2)C(=O)NS(=O)(=O)C1CC1 ; C=C[C@@H]1C[C@]1(NC(=O)[C@@H]1C[C@@H]2CN1C(=O)[C@H](C(C)(C)C)NC(=O)O[C@]1(C)C[C@H]1CCCCc1cccc3c1CN(C3)C(=O)O2)C(=O)NS(=O)(=O)C1CC1 ; C=C[C@@H]1C[C@]1(NC(=O)[C@@H]1C[C@@H]2CN1C(=O)[C@H](C(C)(C)C)NC(=O)O[C@@]1(C)C[C@@H]1CCCCc1cccc3c1CN(C3)C(=O)O2)C(=O)NS(=O)(=O)C1CC1]

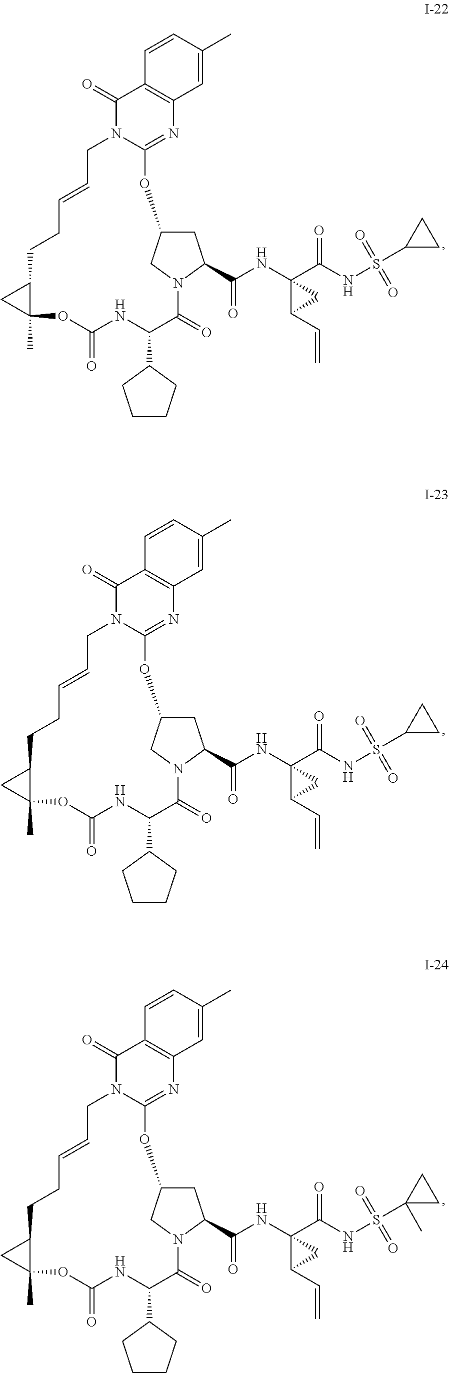 [SMILES: C=C[C@@H]1C[C@]1(NC(=O)[C@@H]1C[C@@H]2CN1C(=O)[C@H](C1CCCC1)NC(=O)O[C@@]1(C)C[C@@H]1CC/C=C/Cn1c(nc3cc(C)ccc3c1=O)O2)C(=O)NS(=O)(=O)C1CC1 ; C=C[C@@H]1C[C@]1(NC(=O)[C@@H]1C[C@@H]2CN1C(=O)[C@H](C1CCCC1)NC(=O)O[C@]1(C)C[C@H]1CC/C=C/Cn1c(nc3cc(C)ccc3c1=O)O2)C(=O)NS(=O)(=O)C1CC1 ; C=C[C@@H]1C[C@]1(NC(=O)[C@@H]1C[C@@H]2CN1C(=O)[C@H](C1CCCC1)NC(=O)O[C@]1(C)C[C@H]1CC/C=C/Cn1c(nc3cc(C)ccc3c1=O)O2)C(=O)NS(=O)(=O)C1(C)CC1]

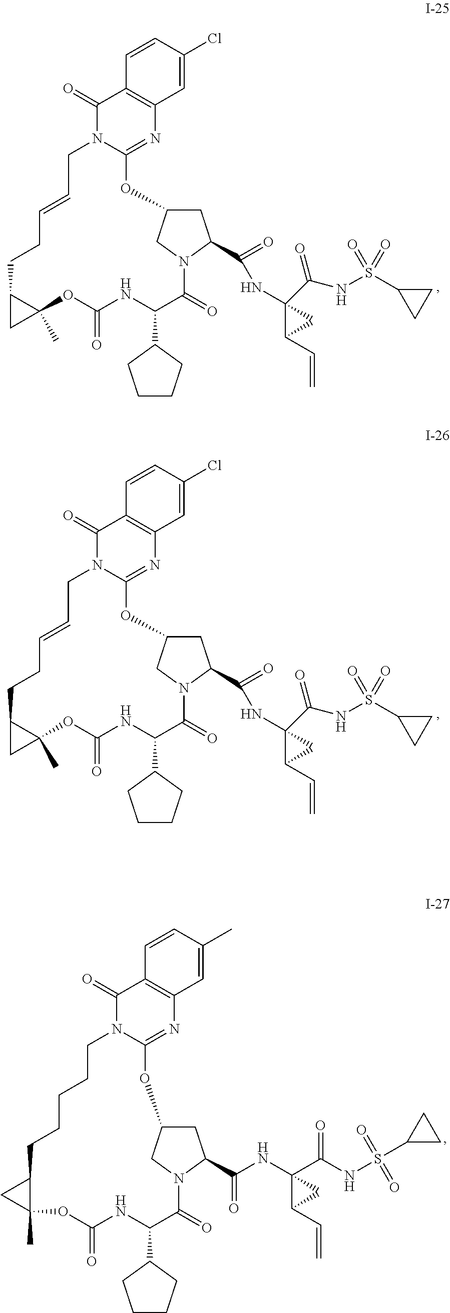 [SMILES: C=C[C@@H]1C[C@]1(NC(=O)[C@@H]1C[C@@H]2CN1C(=O)[C@H](C1CCCC1)NC(=O)O[C@@]1(C)C[C@@H]1CC/C=C/Cn1c(nc3cc(Cl)ccc3c1=O)O2)C(=O)NS(=O)(=O)C1CC1 ; C=C[C@@H]1C[C@]1(NC(=O)[C@@H]1C[C@@H]2CN1C(=O)[C@H](C1CCCC1)NC(=O)O[C@]1(C)C[C@H]1CC/C=C/Cn1c(nc3cc(Cl)ccc3c1=O)O2)C(=O)NS(=O)(=O)C1CC1 ; C=C[C@@H]1C[C@]1(NC(=O)[C@@H]1C[C@@H]2CN1C(=O)[C@H](C1CCCC1)NC(=O)O[C@]1(C)C[C@H]1CCCCCn1c(nc3cc(C)ccc3c1=O)O2)C(=O)NS(=O)(=O)C1CC1]

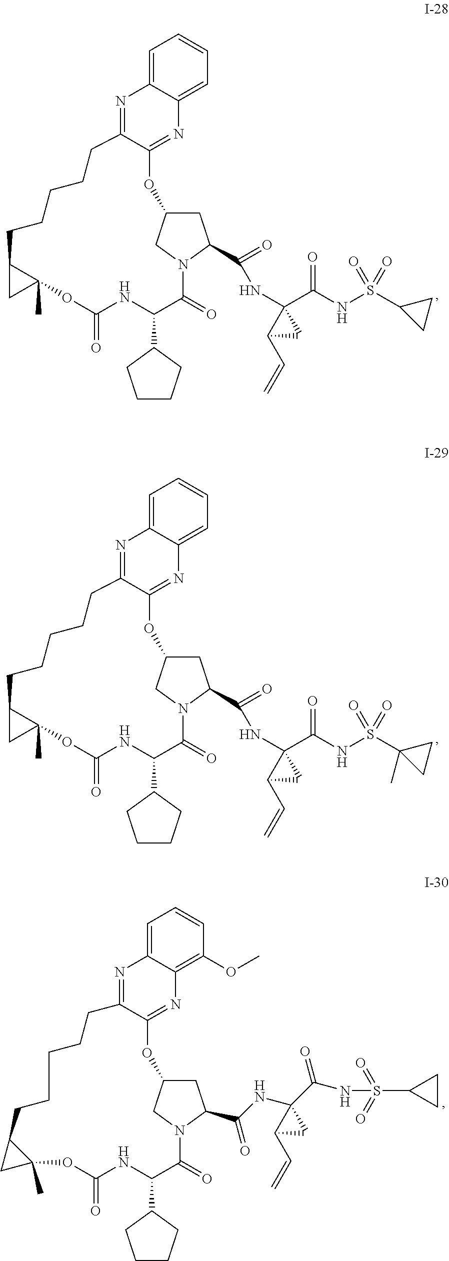 [SMILES: C=C[C@@H]1C[C@]1(NC(=O)[C@@H]1C[C@@H]2CN1C(=O)[C@H](C1CCCC1)NC(=O)O[C@]1(C)C[C@H]1CCCCCc1nc3ccccc3nc1O2)C(=O)NS(=O)(=O)C1CC1 ; C=C[C@@H]1C[C@]1(NC(=O)[C@@H]1C[C@@H]2CN1C(=O)[C@H](C1CCCC1)NC(=O)O[C@]1(C)C[C@H]1CCCCCc1nc3ccccc3nc1O2)C(=O)NS(=O)(=O)C1(C)CC1 ; C=C[C@@H]1C[C@]1(NC(=O)[C@@H]1C[C@@H]2CN1C(=O)[C@H](C1CCCC1)NC(=O)O[C@]1(C)C[C@H]1CCCCCc1nc3cccc(OC)c3nc1O2)C(=O)NS(=O)(=O)C1CC1]

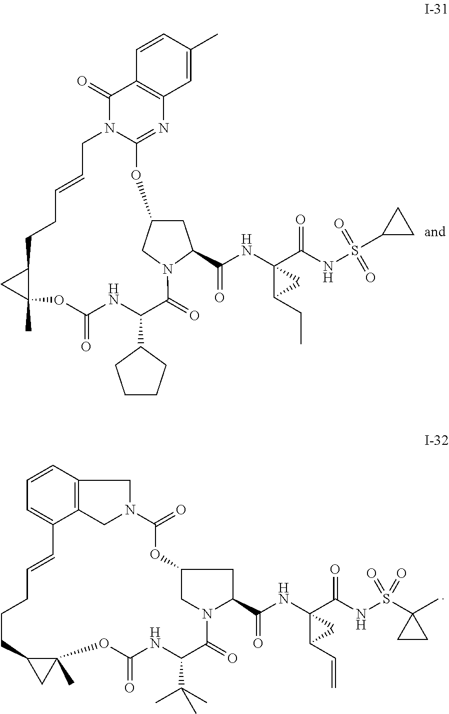 [SMILES: CC[C@@H]1C[C@]1(NC(=O)[C@@H]1C[C@@H]2CN1C(=O)[C@H](C1CCCC1)NC(=O)O[C@]1(C)C[C@H]1CC/C=C/Cn1c(nc3cc(C)ccc3c1=O)O2)C(=O)NS(=O)(=O)C1CC1 ; C=C[C@@H]1C[C@]1(NC(=O)[C@@H]1C[C@@H]2CN1C(=O)[C@H](C(C)(C)C)NC(=O)O[C@]1(C)C[C@H]1CCC/C=C/c1cccc3c1CN(C3)C(=O)O2)C(=O)NS(=O)(=O)C1(C)CC1]

18. A pharmaceutical composition comprising an effective amount of the compound according to claim 1, and a pharmaceutically acceptable carrier.

19. The pharmaceutical composition according to claim 18, further comprising a second therapeutic agent selected from the group consisting of HCV antiviral agents, immunomodulators, and anti-infective agents.

20. The pharmaceutical composition according to claim 18, further comprising a second therapeutic agent selected from the group consisting of HCV protease inhibitors and HCV NS5B polymerase inhibitors.

21-22. (canceled)

23. A method of treating or preventing infection by HCV or of reducing likelihood or severity of symptoms of HCV infection of in a subject in need thereof, said method comprising administering the compound according to claim 1 to a subject in need thereof.
